SEQUENCE LISTING (5 sequences)

SEQ ID NO: 1 - protein, 183 aa, synthetic construct
MRKIVVAAIAVSLTTVSITASASADPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 2 - protein, 119 aa, synthetic construct [sequence omitted]

SEQ ID NO: 3 - protein, 106 aa, synthetic construct [sequence omitted]

SEQ ID NO: 4 - DNA, 32 bp, synthetic construct
  source 1..32: mol_type = other DNA
aattctcctccccaccttccccaccctcccca

SEQ ID NO: 5 - DNA, 156 bp, Homo sapiens
  source 1..156: mol_type = genomic DNA
tataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacata

(19) United States
(12) Patent Application Publication
(10) Pub. No.: US 2026/0199516 A1
(43) Pub. Date: Jul. 16, 2026

(54) COMPOSITIONS AND METHODS FOR DELIVERY OF DNA TO T CELLS

(71) Applicant: FLAGSHIP PIONEERING INNOVATIONS VII, LLC, Cambridge, MA (US)

(72) Inventors: Louisa Marie Helms, Seattle, WA (US); Max Everett Distler, Allston, MA (US); Brian Patrick Mead, Brookline, MA (US); Edward Matthew Kennedy, Bedford, MA (US)

(21) Appl. No.: 19/443,710

(22) Filed: Jan. 8, 2026

Related U.S. Application Data
(60) Provisional application No. 63/743,931, filed on Jan. 10, 2025.
(60) Provisional application No. 63/896,322, filed on Oct. 9, 2025.
(60) Provisional application No. 63/945,105, filed on Dec. 19, 2025.

(51) Int. Cl. A61K 47/69; A61K 31/7105; A61K 31/7115; A61K 31/713; A61K 47/68; A61K 48/00; C07K 16/28; C12N 15/113; C12N 15/88

(52) U.S. Cl. CPC A61K 47/6913; A61K 31/7105; A61K 31/7115; A61K 31/713; A61K 47/6849; A61K 48/0033; C07K 16/2809; C12N 15/113; C12N 15/88; C12N 2310/11

(57) ABSTRACT

The disclosure provides, for example, a targeted lipid nanoparticle composition comprising a lipid nanoparticle, a targeting moiety on the surface of the lipid nanoparticle, wherein the targeting moiety binds a T cell antigen, and a double stranded DNA molecule comprising an effector sequence that encodes an effector. In some embodiments, the targeting moiety comprises an antibody. In some embodiments, the effector comprises a polypeptide. In some embodiments, the double stranded DNA molecule comprises chemically modified nucleobases in its sense strand, and the antisense strand is free of chemically modified nucleobases.

### RELATED APPLICATIONS

This application claims priority to U.S. Ser. No. 63/743,931, filed on Jan. 10, 2025, U.S. Ser. No. 63/896,322, filed on Oct. 9, 2025, and U.S. Ser. No. 63/945,105, filed on Dec. 19, 2025, the entire contents of each of which are incorporated herein by reference.

### SEQUENCE LISTING

The instant application contains a Sequence Listing which has been submitted electronically in XML format and is hereby incorporated by reference in its entirety. Said XML copy, created on Dec. 30, 2025, is named F2128-703310_SL.xml and is 5,748 bytes in size.

### BACKGROUND

There is a need for novel therapeutic modalities to address unmet medical need.

### SUMMARY OF THE INVENTION

Described herein are pharmaceutical compositions, constructs, preparations, methods of using such compositions, constructs and preparations, and methods of making the same.

### Enumerated Embodiments

1. A targeted lipid nanoparticle (tLNP) composition comprising:
a) a lipid nanoparticle (LNP),
b) a targeting moiety on the surface of the LNP, wherein the targeting moiety binds a T cell antigen, and
c) a double stranded DNA molecule comprising an effector sequence that encodes an effector (e.g., a therapeutic effector).
   2. The tLNP composition of embodiment 1, wherein the targeting moiety comprises a polypeptide.
   3. The tLNP composition of embodiment 1 or 2, wherein the targeting moiety comprises an antibody or a ligand.
   4. The tLNP composition of embodiment 3, wherein the antibody comprises a monoclonal antibody.
   5. The tLNP composition of embodiment 3 or 4, wherein the antibody comprises a sdAb, Fab, Fab′, F(ab′)₂, Fv, or scFv.
   6. The tLNP composition of any of the preceding embodiments, wherein the targeting moiety comprises an anti-CD3 antibody molecule.
   7. The tLNP composition of embodiment 6, wherein the CD3 is human CD3.
   8. The tLNP composition of embodiment 6 or 7, wherein the anti-CD3 antibody molecule comprises a heavy chain complementarity determining region 1 (CDR1), heavy chain complementarity determining region 2 (CDR2), and heavy chain complementarity determining region 3 (CDR3) of the amino acid sequence of SEQ ID NO: 2, according to the Kabat definition.
   9. The tLNP composition of any of embodiments 6-8, wherein the anti-CD3 antibody molecule comprises a light chain CDR1, light chain CDR2, and light chain CDR3 of the amino acid sequence of SEQ ID NO: 3, according to the Kabat definition.
   10. The tLNP composition of any of embodiments 6-9, wherein the anti-CD3 antibody molecule comprises a heavy chain variable region comprising the amino acid sequence of SEQ ID NO: 2, or an amino acid sequence with at least 95% identity thereto.
   11. The tLNP composition of any of embodiments 6-10, wherein the anti-CD3 antibody molecule comprises a light chain variable region comprising the amino acid sequence of SEQ ID NO: 3, or an amino acid sequence with at least 95% identity thereto.
   12. The tLNP composition of any of embodiments 6-11, wherein the anti-CD3 antibody molecule comprises a heavy chain variable region comprising the amino acid sequence of SEQ ID NO: 2, or an amino acid sequence with at least 95% identity thereto, and a light chain variable region comprising the amino acid sequence of SEQ ID NO: 3, or an amino acid sequence with at least 95% identity thereto.
   13. The tLNP composition of any of embodiments 6-12, wherein the anti-CD3 antibody molecule comprises a heavy chain variable region comprising the amino acid sequence of SEQ ID NO: 2, and a light chain variable region comprising the amino acid sequence of SEQ ID NO: 3.
   14. The tLNP composition of any of embodiments 1-5, wherein the targeting moiety binds a costimulatory signaling protein (e.g., CD28).
   15. The tLNP composition of any of embodiments 1-5, wherein the targeting moiety comprises an anti-CD5 antibody molecule, an anti-CD7 antibody molecule, an anti-CD28 antibody molecule, an anti-4-1BBL antibody molecule, an anti-CD4 antibody molecule, an anti-CD8 antibody molecule, an anti-CD80 antibody molecule, an anti-CD86 antibody molecule, an anti-CD58 antibody molecule, an anti-CD70 antibody molecule, an anti-SECTM1 antibody molecule, an anti-OX40L antibody molecule, an anti-ICOS-L antibody molecule, an anti-ICAM-1 antibody molecule, an anti-CD2 antibody molecule, an anti-4-1BB antibody molecule, or an OX40 antibody molecule.
   16. The tLNP composition of any of the preceding embodiments, wherein the targeting moiety is connected to the LNP via a click linker.
   17. The tLNP composition of embodiment 16, wherein the click linker comprises a triazole (e.g., a 1,2,3-triazole and/or a disubstituted triazole), a cycloalkene (e.g., a disubstituted alkene), a dihydropyrazine (e.g., a 1,2-dihydropyrazine), a diazole, a sulfur-containing ring (e.g., a thiopyran or a tetrahdrothiophene, e.g., a disubstituted tetrahdrothiophene), or an alkyl sulfide.
   18. The tLNP composition of embodiment 16 or 17, wherein the click linker was formed by or is capable of being formed by a copper-free click chemistry reaction.
   19. The tLNP composition of embodiment 18, wherein the copper-free click chemistry reaction is a strain-promoted azide-alkyne cycloaddition (SPAAC) reaction.
   20. The tLNP composition of embodiment 18 or 19, wherein the copper-free click chemistry reaction comprises a reaction between a dibenzocyclooctyne group (DBCO) and an azide.
   21. The tLNP composition of embodiment 16 or 17, wherein the click linker was formed by a reaction between a tetrazine and a trans-cyclooctene group (TCO).
   22. The tLNP composition of embodiment 16 or 17, wherein the click linker was formed by a reaction between a thiol group and an alkene.
   23. The tLNP composition of any of the preceding embodiments, wherein the DNA molecule is inside the LNP.
   24. The tLNP composition of any of the preceding embodiments, wherein the DNA molecule comprises a promoter sequence operably linked to the effector sequence.
   25. The tLNP composition of any of the preceding embodiments, wherein the effector comprises a polypeptide (e.g., a protein).
   26. The tLNP composition of any of the preceding embodiments, wherein the effector is a chimeric antigen receptor (CAR).
   27. The tLNP composition of embodiment 26, wherein the CAR comprises an antigen binding domain, a transmembrane domain, and a primary signaling domain.
   28. The tLNP composition of embodiment 27, wherein the CAR further comprises a costimulatory domain.
   29. The tLNP composition of any of embodiments 1-24, wherein the effector comprises an RNA (e.g., an mRNA, a tRNA, lncRNA, miRNA, IRNA, snRNA, microRNA, siRNA, piRNA, snoRNA, snRNA, exRNA, scaRNA, Y RNA, or hnRNA), wherein optionally the effector comprises a functional RNA (e.g., a miRNA, siRNA, or tRNA).
   30. The tLNP composition of any of embodiments 1-29, wherein the effector is a mammalian polypeptide, a mammalian RNA, a DNA binding protein, an antigen, an epigenetic modifying factor, a hormone, an enzyme, a nuclease element of a CRISPR system, a mobile genetic element protein, a gene writer, an antibody, a signaling peptide, a receptor ligand, a receptor, or a clotting factor.
   31. The tLNP composition of any of the preceding embodiments, wherein the effector is heterologous to a target cell.
   32. The tLNP composition of any of the preceding embodiments, wherein the DNA molecule is linear.
   33. The tLNP composition of any of embodiments 1-32, wherein the DNA molecule is circular.
   34. The tLNP composition of embodiment 33, wherein the DNA molecule comprises a plasmid or a minicircle.
   35. The tLNP composition of any of embodiments 1-32, wherein the DNA molecule comprises:
(i) an upstream DNA end form which is a closed end;
(ii) a double stranded region; and
(iii) a downstream DNA end form which is a closed end.
   36. The tLNP composition of any of the preceding embodiments, wherein the DNA molecule comprises a chemically modified nucleotide.
   37. The tLNP composition of embodiment 36, wherein the chemically modified nucleotide comprises a chemically modified nucleobase.
   38. The tLNP composition of embodiment 36, wherein the chemically modified nucleotide comprises a chemically modified sugar.
   39. The tLNP composition of embodiment 36, wherein the chemically modified nucleotide comprises a backbone modification (e.g., a phosphorothioate linkage).
   40. The tLNP composition of embodiment 37, wherein the chemically modified nucleobase comprises a uracil nucleobase.
   41. The tLNP composition of embodiment 40, wherein at least 1%, at least 5%, at least 10%, at least 15%, at least 20%, at least 25%, at least 50%, at least 75%, at least 90%, at least 95%, at least 96%, at least 97%, at least 98%, at least 99%, or all of thymine or uracil positions in the sense strand of the DNA molecule comprise a uracil nucleobase.
   42. The tLNP composition of embodiment 40 or 41, wherein 1%-100% (e.g., 1%-5%, 5%-10%, 10%-15%, 15%-20%, 20%-25%, 25%-30%, 30%-35%, 35%-40%, 40%-45%, 45%-50%, 50%-55%, 55%-60%, 60%-65%, 65%-70%, 70%-75%, 75%-80%, 80%-85%, 85%-90%, 90%-95%, 95%-96%, 96%-97%, 97%-98%, 98%-99%, 99%-100%, or 95%-100%) of thymine or uracil positions in the sense strand of the DNA molecule comprise a uracil nucleobase.
   43. The tLNP composition of any of embodiments 40-42, wherein every thymine or uracil position in a stretch of at least 100, at least 200, at least 300, at least 400, at least 500, at least 600, at least 700, at least 800, at least 900, at least 1000, at least 1500, or at least 2000 nucleotides in the sense strand of the DNA molecule comprises a uracil nucleobase.
   44. The tLNP composition of any of embodiments 40-43, wherein every thymine or uracil position in a stretch of 100-200, 200-300, 300-400, 400-500, 500-600, 600-700, 700-800, 800-900, 900-1000, 1000-1500, or 1500-2000 nucleotides in the sense strand of the DNA molecule comprises a uracil nucleobase.
   45. The tLNP composition of any of embodiments 40-44, wherein the uracil nucleobase is a canonical uracil nucleobase or a chemically modified uracil nucleobase.
   46. The tLNP composition of any of embodiments 40-45, wherein the uracil nucleobase is a canonical uracil nucleobase.
   47. The tLNP composition of embodiment 46, wherein at least 80%, at least 85%, at least 90%, at least 95%, or all of the chemically modified nucleobases of the DNA molecule comprise a canonical uracil nucleobase.
   48. The tLNP composition of embodiment 46 or 47, wherein 80%-85%, 85%-90%, 90%-95%, or 95%-100% of the chemically modified nucleobases of the DNA molecule comprise a canonical uracil nucleobase.
   49. The tLNP composition of any of embodiments 46-48, wherein at least 1%, at least 5%, at least 10%, at least 15%, at least 20%, at least 25%, at least 50%, at least 75%, at least 90%, at least 95%, at least 96%, at least 97%, at least 98%, at least 99%, or all of thymine or uracil positions in the sense strand of the DNA molecule comprise a canonical uracil nucleobase.
   50. The tLNP composition of any of embodiments 46-49, wherein 1%-100% (e.g., 1%-5%, 5%-10%, 10%-15%, 15%-20%, 20%-25%, 25%-30%, 30%-35%, 35%-40%, 40%-45%, 45%-50%, 50%-55%, 55%-60%, 60%-65%, 65%-70%, 70%-75%, 75%-80%, 80%-85%, 85%-90%, 90%-95%, 95%-96%, 96%-97%, 97%-98%, 98%-99%, 99%-100%, or 95%-100%) of thymine or uracil positions in the sense strand of the DNA molecule comprise a canonical uracil nucleobase.
   51. The tLNP composition of any of embodiments 40-45, wherein the uracil nucleobase is a chemically modified uracil nucleobase.
   52. The tLNP composition of embodiment 51, wherein at least 1%, at least 5%, at least 10%, at least 15%, at least 20%, at least 25%, at least 50%, at least 75%, at least 90%, at least 95%, at least 96%, at least 97%, at least 98%, at least 99%, or all of thymine or uracil positions in the sense strand of the DNA molecule comprise a chemically modified uracil nucleobase.
   53. The tLNP composition of embodiment 51 or 52, wherein 1%-100% (e.g., 1%-5%, 5%-10%, 10%-15%, 15%-20%, 20%-25%, 25%-30%, 30%-35%, 35%-40%, 40%-45%, 45%-50%, 50%-55%, 55%-60%, 60%-65%, 65%-70%, 70%-75%, 75%-80%, 80%-85%, 85%-90%, 90%-95%, 95%-96%, 96%-97%, 97%-98%, 98%-99%, 99%-100%, or 95%-100%) of thymine or uracil positions in the sense strand of the DNA molecule comprise a chemically modified uracil nucleobase.
   54. The tLNP composition of any of embodiments 51-53, wherein the chemically modified uracil nucleobase comprises 5-hydroxymethyluracil.
   55. The tLNP composition of embodiment 54, wherein at least 80%, at least 85%, at least 90%, at least 95%, or all of the chemically modified nucleobases of the DNA molecule comprise 5-hydroxymethyluracil.
   56. The tLNP composition of embodiment 54 or 55, wherein 80%-85%, 85%-90%, 90%-95%, or 95%-100% of the chemically modified nucleobases of the DNA molecule comprise 5-hydroxymethyluracil.
   57. The tLNP composition of any of embodiments 54-56, wherein at least 1%, at least 5%, at least 10%, at least 15%, at least 20%, at least 25%, at least 50%, at least 75%, at least 90%, at least 95%, at least 96%, at least 97%, at least 98%, at least 99%, or all of thymine or uracil positions in the sense strand of the DNA molecule comprise 5-hydroxymethyluracil.
   58. The tLNP composition of any of embodiments 54-57, wherein 1%-100% (e.g., 1%-5%, 5%-10%, 10%-15%, 15%-20%, 20%-25%, 25%-30%, 30%-35%, 35%-40%, 40%-45%, 45%-50%, 50%-55%, 55%-60%, 60%-65%, 65%-70%, 70%-75%, 75%-80%, 80%-85%, 85%-90%, 90%-95%, 95%-96%, 96%-97%, 97%-98%, 98%-99%, 99%-100%, or 95%-100%) of thymine or uracil positions in the sense strand of the DNA molecule comprise 5-hydroxymethyluracil.
   59. The tLNP composition of any of the preceding embodiments, wherein the DNA molecule comprises a first type of chemically modified nucleobase and a second type of chemically modified nucleobase.
   60. The tLNP composition of any of the preceding embodiments, wherein the DNA molecule comprises a sense strand and an antisense strand, and wherein the antisense strand comprises one or more chemically modified nucleobases.
   61. The tLNP composition of any of embodiments 1-59, wherein the DNA molecule comprises a sense strand and an antisense strand, and wherein the antisense strand is substantially free of (e.g., is free of) chemically modified nucleobases.
   62. The tLNP composition of embodiment 60 or 61, wherein the sense strand is substantially free of (e.g., is free of) any chemically modified nucleobases.
   63. The tLNP composition of embodiment 60 or 61, wherein the sense strand comprises one or more chemically modified nucleobases.
   64. The tLNP composition of any of embodiments 1-59, wherein the DNA molecule comprises a sense strand and an antisense strand, wherein the sense strand comprises one or more chemically modified nucleobases, and wherein the antisense strand is substantially free of (e.g., is free of) chemically modified nucleobases.
   65. The tLNP composition of any of embodiments 1-61, 63, or 64, wherein the sense strand of the promoter sequence comprises one or more chemically modified nucleobases.
   66. The tLNP composition of any of embodiments 1-64, wherein the sense strand of the promoter sequence is substantially free of (e.g., is free of) chemically modified nucleobases.
   67. The tLNP composition of any of the preceding embodiments, wherein the DNA molecule comprises one or more sequences encoding a 5′ untranslated region (5′ UTR) that is 5′ of the effector sequence and/or a 3′ untranslated region (3′ UTR) that is 3′ of the effector sequence.
   68. The tLNP composition of embodiment 67, wherein the sense strand of said one or more sequences encoding the 5′ UTR and/or 3′ UTR comprises one or more chemically modified nucleobases.
   69. The tLNP composition of embodiment 67, wherein the sense strand of said one or more sequences encoding the 5′ UTR and/or 3′ UTR is substantially free of (e.g., is free of) chemically modified nucleobases.
   70. The tLNP composition of any of the preceding embodiments, wherein the DNA molecule comprises a sequence encoding a polyadenylation site and polyadenylation signal.
   71. The tLNP composition of embodiment 70, wherein the sense strand of said sequence encoding a polyadenylation site and polyadenylation signal comprises one or more chemically modified nucleobases.
   72. The tLNP composition of embodiment 70, wherein the sense strand of said sequence encoding a polyadenylation site and polyadenylation signal is substantially free of (e.g., is free of) chemically modified nucleobases.
   73. The tLNP composition of any of embodiments 1-61 or 63-72, wherein the sense strand of the effector sequence comprises one or more chemically modified nucleobases.
   74. The tLNP composition of any of the embodiments 1-72, wherein the sense strand of the effector sequence is substantially free of (e.g., is free of) chemically modified nucleobases.
   75. The tLNP composition of any of the preceding embodiments, wherein the DNA molecule comprises an intron sequence.
   76. The tLNP composition of embodiment 75, wherein the sense strand of said intron sequence comprises one or more chemically modified nucleobases.
   77. The tLNP composition of embodiment 75, wherein the sense strand of said intron sequence is substantially free of (e.g., is free of) chemically modified nucleobases.
   78. The tLNP composition of any of the preceding embodiments, wherein the DNA molecule comprises an enhancer sequence.
   79. The tLNP composition of embodiment 78, wherein the sense strand of said enhancer sequence comprises one or more chemically modified nucleobases.
   80. The tLNP composition of embodiment 78, wherein the sense strand of said enhancer sequence is substantially free of (e.g., is free of) chemically modified nucleobases.
   81. The tLNP composition of any of the preceding embodiments, wherein the sense strand comprises one or more backbone modifications, e.g., phosphorothioate linkages.
   82. The tLNP composition of embodiment 81, wherein said one or more backbone modifications of the sense strand are situated in a stretch of adjacent nucleotides, wherein optionally the stretch of adjacent nucleotides is 4, 6, 8, 10, 12, 14, or 16 nucleotides in length.
   83. The tLNP composition of any of the preceding embodiments, wherein the sense strand comprises a region that is substantially free of (e.g., is free of) backbone modifications.
   84. The tLNP composition of embodiment 83, wherein the region is at least 50, at least 100, at least 200, at least 300, at least 400, at least 500, or at least 1,000 nucleotides in length.
   85. The tLNP composition of embodiment 83 or 84, wherein the region is 50-100, 100-200, 200-300, 300-400, 400-500, 500-600, 600-700, 700-800, 800-900, 900-1000, 1000-1500, or 1500-2000 nucleotides in length.
   86. The tLNP composition of any of the preceding embodiments, wherein the antisense strand is substantially free of (e.g., is free of) phosphorothioate linkages.
   87. The tLNP composition of any of the preceding embodiments, wherein the antisense strand is substantially free of (e.g., is free of) backbone modifications.
   88. The tLNP composition of any of embodiments 1-61 or 63-87, wherein at least 1%, at least 5%, at least 10%, at least 15%, at least 20%, or at least 25% of positions in the sense strand of the DNA molecule comprise chemically modified nucleobases.
   89. The tLNP composition of any of embodiments 1-61 or 63-88, wherein at least 1%, at least 5%, at least 10%, at least 15%, at least 20%, or at least 25% of positions in the sense strand of the DNA molecule comprise the same chemically modified nucleobase.
   90. The tLNP composition of any of embodiments 1-61 or 63-89, wherein 1%-25% (e.g., 1%-5%, 5%-10%, 10%-15%, 15%-20%, or 20%-25%) of positions in the sense strand of the DNA molecule comprise chemically modified nucleobases.
   91. The tLNP composition of any of embodiments 1-61 or 63-90, wherein 1%-25% (e.g., 1%-5%, 5%-10%, 10%-15%, 15%-20%, or 20%-25%) of positions in the sense strand of the DNA molecule comprise the same chemically modified nucleobase.
   92. The tLNP composition of any of embodiments 37 or 40-91, wherein at least 80%, at least 85%, at least 90%, at least 95%, or all of the chemically modified nucleobases of the DNA molecule have the same chemical structure.
   93. The tLNP composition of any of embodiments 37 or 40-92, wherein 80%-85%, 85%-90%, 90%-95%, or 95%-100% of the chemically modified nucleobases of the DNA molecule have the same chemical structure.
   94. The tLNP composition of any of the preceding embodiments, wherein the longest stretch of unmodified nucleotides in the sense strand is no more than 1000, no more than 900, no more than 800, no more than 700, no more than 600, no more than 500, no more than 400, no more than 300, no more than 200, no more than 100, no more than 50, or no more than 10 nucleotides.
   95. The tLNP composition of any of the preceding embodiments, wherein the longest stretch of unmodified nucleotides in the sense strand is 10-50, 50-100, 100-200, 200-300, 300-400, 400-500, 500-600, 600-700, 700-800, 800-900, or 900-1000 nucleotides.
   96. The tLNP composition of any of the preceding embodiments, wherein the longest stretch of unmodified nucleobases in the sense strand is no more than 1000, no more than 900, no more than 800, no more than 700, no more than 600, no more than 500, no more than 400, no more than 300, no more than 200, no more than 100, no more than 50, or no more than 10 nucleobases.
   97. The tLNP composition of any of the preceding embodiments, wherein the longest stretch of unmodified nucleobases in the sense strand is 10-50, 50-100, 100-200, 200-300, 300-400, 400-500, 500-600, 600-700, 700-800, 800-900, or 900-1000 nucleobases.
   98. The tLNP composition of any of the preceding embodiments, wherein the antisense strand comprises no more than 10, no more than 9, no more than 8, no more than 7, no more than 6, no more than 5, no more than 4, no more than 3, no more than 2, or no more than 1 chemically modified nucleotides.
   99. The tLNP composition of any of the preceding embodiments, wherein the antisense strand comprises no more than 10, no more than 9, no more than 8, no more than 7, no more than 6, no more than 5, no more than 4, no more than 3, no more than 2, or no more than 1 chemically modified nucleobases.
   100. The tLNP composition of any of the preceding embodiments, wherein the antisense strand comprises no more than 10, no more than 9, no more than 8, no more than 7, no more than 6, no more than 5, no more than 4, no more than 3, no more than 2, or no more than 1 backbone modifications.
   101. The tLNP composition of any of the preceding embodiments, wherein the antisense strand comprises no more than 10, no more than 9, no more than 8, no more than 7, no more than 6, no more than 5, no more than 4, no more than 3, no more than 2, or no more than 1 nucleotides having a chemically modified sugar.
   102. The tLNP composition of any of the preceding embodiments, wherein each of the sense and antisense strands of the DNA molecule comprises one or more chemically modified nucleotides.
   103. The tLNP composition of any of the preceding embodiments, wherein each of the sense and antisense strands of the DNA molecule comprises one or more phosphorothioate linkages.
   104. The tLNP composition of any of the preceding embodiments, wherein the DNA molecule has a length of at least 500, at least 1000, at least 2000, at least 3000, at least 4000, at least 5000, at least 6000, at least 7000, at least 8000, at least 9000, at least 10000, at least 11000, or at least 12000 nucleotides.
   105. The tLNP composition of any of the preceding embodiments, wherein the DNA molecule has a length of between 500-1000, 1000-2000, 2000-3000, 3000-4000, 4000-5000, 5000-6000, 6000-7000, 7000-8000, 8000-9000, 9000-10000, 10000-11000, or 11000-12000 nucleotides.
   106. The tLNP composition of any of the preceding embodiments, wherein the DNA molecule has a length of at least 15 nucleotides, at least 30 nucleotides, at least 50 nucleotides, at least 75 nucleotides, 100 nucleotides, at least 200 nucleotides, at least 300 nucleotides, at least 500 nucleotides, at least 750 nucleotides, at least 1,000 nucleotides, at least 2,000 nucleotides, at least 3,000 nucleotides, at least 4,000 nucleotides, at least 5,000 nucleotides, at least 6,000 nucleotides, at least 7,000 nucleotides, at least 8,000 nucleotides, at least 9,000 nucleotides, at least 10,000 nucleotides, at least 11,000 nucleotides, at least 12,000 nucleotides, at least 15,000 nucleotides, at least 20,000 nucleotides, at least 25,000 nucleotides, at least 30,000 nucleotides, at least 35,000 nucleotides, at least 40,000 nucleotides at least 45,000 nucleotides, at least 50,000 nucleotides, at least 60,000 nucleotides, or more.
   107. The tLNP composition of any of the preceding embodiments, wherein the DNA molecule has a length of between 20 and 1000 nucleotides, between 20 and 50 nucleotides, between 100 and 500 nucleotides, between 500 and 50,000 nucleotides, between 1,000 and 50,000 nucleotides, between 2,000 and 40,000 nucleotides, between 5,000 and 50,000 nucleotides, between 500 and 50,000 nucleotides, between 500 and 25,000 nucleotides, between 1,000 and 20,000 nucleotides, between 1,000 and 10,000 nucleotides, between 10,000 and 60,000 nucleotides, between 1,000 and 20,000 nucleotides, between 1,000 and 40,000 nucleotides, between 500 and 1000 nucleotides, between 1000 and 2,000 nucleotides, between 2,000 and 3,000 nucleotides, between 3,000 and 4,000 nucleotides, between 4,000 and 5,000 nucleotides, between 5,000 and 6,000 nucleotides, between 6,000 and 7,000 nucleotides, between 7,000 and 8,000 nucleotides, between 8,000 and 9,000 nucleotides, between 9,000 and 10,000 nucleotides, between 10,000 and 11,000 nucleotides, or between 11,000 and 12,000 nucleotides.
   108. The tLNP composition of any of the preceding embodiments, wherein the DNA molecule is resistant to endonuclease digestion.
   109. The tLNP composition of any of the preceding embodiments, wherein the DNA molecule is resistant to immune sensor recognition.
   110. The tLNP composition of any of the preceding embodiments, wherein at least 80%, at least 85%, at least 90%, at least 95%, at least 98%, at least 99%, or 100% of the sugars of the DNA molecule are deoxyribose sugars.
   111. The tLNP composition of any of the preceding embodiments, wherein all positions in the DNA molecule comprise a deoxyribose sugar.
   112. The tLNP composition of any of the preceding embodiments, wherein all positions in the sense strand of the DNA molecule comprise a deoxyribose sugar.
   113. The tLNP composition of any of the preceding embodiments, wherein all positions in the antisense strand of the DNA molecule comprise a deoxyribose sugar.
   114. The tLNP composition of any of the preceding embodiments, wherein the DNA molecule comprises a chemical modification of a phosphate group.
   115. The tLNP composition of any of the preceding embodiments, wherein the DNA molecule comprises a chemically modified sugar, e.g., a 2′-deoxy-2′-fluoro (2′-F) nucleotide or a 2′-O-methyl(2′-O-Me) nucleotide.
   116. The tLNP composition of any of the preceding embodiments, wherein the DNA molecule further comprises one or more additional chemically modified nucleotide, wherein the additional chemically modified nucleotide comprises a modification in the backbone, sugar, or nucleobase.
   117. The tLNP composition of any of the preceding embodiments, wherein one or more of the chemically modified nucleotides is conjugated to a peptide or protein.
   118. The tLNP composition of any of the preceding embodiments, wherein the DNA molecule further comprises one or more of:
i) a heterologous functional sequence, e.g., a nuclear targeting sequence or a regulatory sequence;
ii) a maintenance sequence; and/or
iii) an origin of replication.
   119. The tLNP composition of embodiment 118, wherein the nuclear targeting sequence comprises a CT3 sequence (e.g., a sequence of AATTCTCCTCCCCACCTTCCCCACCCTCCCCA (SEQ ID NO: 4)), or a nucleic acid sequence having at least 75%, at least 80%, at least 85%, at least 90%, at least 95%, at least 96%, at least 97%, at least 98%, or at least 99% sequence identity thereto.
   120. The tLNP composition of embodiment 118, wherein the nuclear targeting sequence binds to a hnRNPK protein (e.g., a human hnRNPK protein).
   121. The tLNP composition of any of the preceding embodiments, wherein the DNA molecule can be replicated (e.g., by a DNA polymerase native to a cell comprising the DNA molecule).
   122. The tLNP composition of any of embodiments 1-120, wherein the DNA molecule cannot be replicated.
   123. The tLNP composition of any of the preceding embodiments, wherein the DNA molecule lacks a material portion of vector backbone (e.g., plasmid backbone).
   124. The tLNP composition of any of the preceding embodiments, wherein the DNA molecule does not comprise a non-human (e.g., bacterial) origin of replication.
   125. The tLNP composition of any of the preceding embodiments, wherein the DNA molecule does not comprise an antibiotic resistance selectable marker.
   126. The tLNP composition of any of the preceding embodiments, wherein the DNA molecule is not supercoiled.
   127. The tLNP composition of any of the preceding embodiments, wherein the effector sequence does not encode a viral protein.
   128. The tLNP composition of any of the preceding embodiments, wherein the LNP comprises a cationic lipid (e.g., an ionizable lipid), a non-cationic lipid (e.g., phospholipid), a structural lipid (e.g., cholesterol), or a PEG-modified lipid.
   129. The tLNP composition of any of the preceding embodiments, wherein the LNP comprises a second lipid.
   130. The tLNP composition of embodiment 129, wherein the second lipid comprises a cationic lipid, a non-cationic (e.g., neutral, anionic, or zwitterionic) lipid, or an ionizable lipid.
   131. The tLNP composition of any of the preceding embodiments, which has a nitrogen:phosphate (N/P) ratio of 3-9, e.g., 4-8 or 5-7, e.g., about 6.
   132. The tLNP composition of any of the preceding embodiments, wherein the DNA molecule does not comprise a viral packaging signal.
   133. The tLNP composition of any of the preceding embodiments, wherein the DNA molecule does not comprise a viral origin of replication.
   134. The tLNP composition of any of the preceding embodiments, wherein the DNA molecule does not encode a viral protein.
   135. The tLNP composition of any of the preceding embodiments, wherein the DNA molecule does not encode a viral capsid.
   136. The tLNP composition of any of the preceding embodiments, wherein the DNA molecule does not comprise a viral ITR.
   137. The tLNP composition of any of the preceding embodiments, which further comprises an RNA molecule (e.g., mRNA molecule), optionally wherein:
(a) the LNP comprises both the DNA molecule and the RNA molecule, or
(b) the tLNP composition comprises:
   (i) a first population of LNPs that comprises the DNA molecule and does not comprise the RNA molecule, and
   (ii) a second population of LNPs that comprises the RNA molecule and does not comprise the DNA molecule.
      138. The tLNP composition of embodiment 137, wherein the RNA molecule comprises a second effector sequence that encodes a second effector (e.g., a therapeutic effector).
      139. The tLNP composition of embodiment 138, wherein the second effector sequence comprised by the RNA molecule has a different sequence than the effector sequence comprised by the DNA molecule.
      140. The tLNP composition of embodiment 138, wherein the second effector sequence comprised by the RNA molecule has the same sequence as the effector sequence comprised by the DNA molecule.
      141. A pharmaceutical composition comprising the tLNP composition of any of embodiments 1-140.
      142. The pharmaceutical composition of embodiment 141, which comprises a plurality of the DNA molecules comprising the effector sequence.
      143. The pharmaceutical composition of embodiment 141 or 142, which comprises at least 0.5 mg, at least 1 mg, at least 2 mg, at least 5 mg, at least 10 mg, at least 20 mg, or at least 50 mg of the DNA molecules.
      144. The pharmaceutical composition of any of embodiments 141-143, which comprises 0.5-1, 1-2, 2-5, 5-10, 10-20, 20-50, or 50-100 mg of the DNA molecules.
      145. The pharmaceutical composition of any of embodiments 142-144, wherein:
at least 50%, at least 60%, or at least 70% of the DNA molecules in the plurality have substantially the same length;
at least 50%, at least 60%, or at least 70% of the DNA molecules in the plurality have a length in a predetermined range; or at least 50%, at least 60%, or at least 70% of the DNA molecules in the plurality have a length of between 100, 200, 300, 400, or 500 nucleotides of each other.
   146. The tLNP composition or pharmaceutical composition of any of the preceding embodiments, wherein the DNA molecule is unencapsidated.
   147. The tLNP composition or pharmaceutical composition of any of the preceding embodiments, which is substantially free of, e.g., is free of, microorganisms.
   148. The tLNP composition or pharmaceutical composition of any of the preceding embodiments, which is substantially free of, e.g., is free of, bacteria.
   149. The tLNP composition or pharmaceutical composition of any of the preceding embodiments, which is substantially free of, e.g., is free of, virus.
   150. The tLNP composition or pharmaceutical composition of any of the preceding embodiments, which is substantially free of, e.g., is free of, proteins.
   151. The tLNP composition or pharmaceutical composition of any of the preceding embodiments, which is substantially free of, e.g., is free of, viral proteins.
   152. The pharmaceutical composition of any of embodiments 141-151, further comprising an electroporation buffer.
   153. The pharmaceutical composition of any of embodiments 141-152, further comprising a transfection reagent.
   154. A method of introducing a DNA molecule and/or an RNA molecule into a T cell, the method comprising:
(a) providing a cell population comprising T cells;
(b) contacting the T cell with the tLNP composition or pharmaceutical composition of any of the preceding embodiments,
thereby introducing the DNA molecule and/or the RNA molecule into the T cell.
   155. The method of embodiment 154, wherein the T cells proliferate after the contacting of (b).
   156. The method of embodiment 154 or 155, wherein the DNA molecule comprises a chemically modified nucleobase.
   157. The method of embodiment 156, wherein the DNA molecule:
(i) is double stranded and circular, and
(ii) comprises a sense strand and an antisense strand, wherein the sense strand comprises the chemically modified nucleobase.
   158. The method of embodiment 156 or 157, wherein the chemically modified nucleobase comprises a canonical uracil nucleobase or 5-hydroxymethyluracil.
   159. The method of any of embodiments 156-158, wherein proliferation of the T cells is increased relative to proliferation of a control cell population comprising T cells contacted with a control tLNP composition having the same LNP, same targeting moiety, and same sequence of DNA molecule, but the DNA molecule lacks chemically modified nucleobases.
   160. The method of any of embodiments 154-159, wherein the method results in detectable expression of the effector, e.g., at 2 days or 4 days after the contacting of (b).
   161. The method of any of embodiments 156-160, wherein the method results in detectable expression of the effector at a level at least 40%, at least 50%, at least 60%, at least 70%, or at least 80% relative to the expression of the effector in a control cell population comprising T cells contacted with a control tLNP composition having the same LNP, same targeting moiety, and same sequence of DNA molecule, but the DNA molecule lacks chemically modified nucleobases.
   162. The method of any of embodiments 156-162, wherein the method results in detectable expression of the effector at a level that is 40-50%, 50%-60%, 60%-70%, or 70%-80% of the expression of the effector in a control cell population comprising T cells contacted with a control tLNP composition having the same LNP, same targeting moiety, and same sequence of DNA molecule, but the DNA molecule lacks chemically modified nucleobases.
   163. The method of any of embodiments 154-162, wherein the effector is expressed in at least 0.1%, at least 0.5%, at least 1%, at least 5%, at least 10%, at least 15%, at least 20%, at least 25%, at least 30%, or at least 35% of the T cells in the cell population.
   164. The method of any of embodiments 154-163, wherein the effector is expressed in 0.1%-0.5%, 0.5%-1%, 1%-5%, 5%-10%, 10%-15%, 15%-20%, 20%-25%, 25%-30%, or 30%-35% of the T cells in the cell population.
   165. The method of any of embodiments 156-164, wherein the method results in a level of cyclic AMP-GMP (cGAMP) in the cell population comprising T cells that is less than 60%, less than 50%, less than 40%, less than 30%, less than 20%, less than 10%, less than 5%, or less than 1% of the level of cGAMP in a control cell population comprising T cells contacted with a control tLNP composition having the same LNP, same targeting moiety, and same sequence of DNA molecule, but the DNA molecule lacks chemically modified nucleobases, e.g., as measured using an ELISA.
   166. The method of any of embodiments 156-165, wherein the method results in a level of cGAMP in the cell population comprising T cells that is 1%-5%, 5%-10%, 10%-20%, 20%-30%, 30%-40%, 40%-50%, or 50%-60% of the level of cGAMP in a control cell population comprising T cells contacted with a control tLNP composition having the same LNP, same targeting moiety, and same sequence of DNA molecule, but the DNA molecule lacks chemically modified nucleobases, e.g., as measured using an ELISA.
   167. The method of any of embodiments 156-166, wherein the method results in a level of IFN-λ protein in the cell population comprising T cells that is lower than the level of IFN-λ protein in a control cell population comprising T cells contacted with a control tLNP composition having the same LNP, same targeting moiety, and same sequence of DNA molecule, but the DNA molecule lacks chemically modified nucleobases.
   168. The method of any of embodiments 156-167, wherein the method results in a level of IP-10 protein in the cell population comprising T cells that is lower than the level of IP-10 protein in a control cell population comprising T cells contacted with a control tLNP composition having the same LNP, same targeting moiety, and same sequence of DNA molecule, but the DNA molecule lacks chemically modified nucleobases.
   169. The method of any of embodiments 154-168, wherein the cell population comprises one or more cells other than T cells.
   170. The method of any of embodiments 154-169, wherein the cell population is from a subject, e.g., a subject having, or identified as having, a cancer.
   171. The method of any of embodiments 154-170, wherein the cell population is from a subject having, or identified as having, an autoimmune disease.
   172. The method of any of embodiments 154-171, wherein the T cells are human T cells.
   173. The method of any of embodiments 154-172, wherein the tLNP composition is contacted with the T cell at a final concentration of between 1 μg DNA/mL and 20 μg DNA/mL, e.g., between 2 μg DNA/mL and 16 μg DNA/mL, e.g., 2 μg DNA/mL, 8 μg DNA/mL, or 16 μg DNA/mL.
   174. The method of any of embodiments 154-173, wherein the method is performed in vitro.
   175. The method of any of embodiments 154-158, wherein the method is performed in vivo.
   176. A method of treating a disease or disorder in a subject, the method comprising:
administering to the subject the tLNP composition or pharmaceutical composition of any of embodiments 1-153;
thereby treating the disease or disorder.
   177. A method of treating a cancer in a subject, the method comprising:
administering to the subject the tLNP composition or pharmaceutical composition of any of embodiments 1-153;
thereby treating the cancer.
   178. The method of embodiment 170 or 177, wherein the cancer is a hematological cancer.
   179. The method of any of embodiments 170, 177, or 178, wherein the cancer is a lymphoma (e.g., non-Hodgkin lymphoma (NHL)), a leukemia, or multiple myeloma.
   180. The method of any of embodiments 170 or 177-179, wherein the cancer is a diffuse large B-cell lymphoma (DLBCL), mantle cell lymphoma (MCL), follicular lymphoma, acute lymphoblastic leukemia (ALL), chronic lymphocytic leukemia (CLL), small lymphocytic lymphoma (SLL), or acute myeloid leukemia (AML).
   181. The method of embodiment 170 or 177, wherein the cancer is a solid tumor.
   182. A method of treating an autoimmune disease in a subject, the method comprising:
administering to the subject the tLNP composition or pharmaceutical composition of any of embodiments 1-153;
thereby treating the autoimmune disease.
   183. The method of embodiment 171 or 182, wherein the autoimmune disease is lupus (e.g., systemic lupus erythematosus (SLE)), systemic sclerosis, idiopathic inflammatory myopathy, dermatomyositis, or myasthenia gravis.
   184. A method of expressing an effector and/or a second effector in a target cell, the method comprising:
(i) introducing into a target cell the tLNP composition or pharmaceutical composition of any of embodiments 1-153; and
(ii) maintaining (e.g., incubating) the cell under conditions suitable for expressing the effector from the DNA molecule and/or the second effector from the RNA molecule; thereby expressing the effector and/or the second effector in the target cell.
   185. A method of delivering an effector and/or a second effector to a target cell, the method comprising:
introducing into a target cell the tLNP composition or pharmaceutical composition of any of embodiments 1-153;
thereby delivering the effector and/or the second effector to the target cell.
   186. A method of delivering a DNA molecule and/or an RNA molecule to a target cell, the method comprising:
introducing into a target cell the tLNP composition or pharmaceutical composition of any of embodiments 1-153;
thereby delivering the DNA molecule and/or the RNA molecule to the target cell.
   187. A method of modulating (e.g., increasing or decreasing) a biological activity in a target cell, the method comprising:
(i) providing a target cell the tLNP composition or pharmaceutical composition of any of embodiments 1-153, wherein the DNA molecule comprises a sequence encoding an effector that modulates a biological activity in the target cell and/or wherein the RNA molecule comprises a sequence encoding a second effector that modulates a biological activity in the target cell; and
(ii) maintaining (e.g., incubating) the cell under conditions suitable for expressing the effector from the DNA molecule and/or the second effector from the RNA molecule;
thereby modulating the biological activity in the target cell.
   188. The method of embodiment 187, wherein the biological activity comprises cell growth, cell metabolism, cell signaling, cell movement, specialization, interactions, division, transport, homeostasis, osmosis, or diffusion.
   189. The method of embodiment 187 or 188, wherein the cell is an animal cell, e.g., a mammalian cell, e.g., a human cell.
   190. A method of treating a cell, tissue, or subject in need thereof, the method comprising:
administering to the cell, tissue, or subject the tLNP composition or pharmaceutical composition of any of embodiments 1-153;
thereby treating the cell, tissue, or subject.
   191. The method of any of embodiments 176-190, which is performed ex vivo or in vivo.
   192. The method of any of embodiments 154-191, which further comprises contacting the T cell or target cell with a second agent, which is an activating agent (e.g., an agent comprising an anti-CD3 antibody and/or an anti-CD28 antibody).
   193. The method of any of embodiments 154-191, which does not comprise contacting the T cell or target cell with an activating agent (e.g., an agent comprising an anti-CD3 antibody and/or an anti-CD28 antibody).
   194. A method of making a tLNP composition, the method comprising:
(a) providing a double stranded DNA molecule;
(b) contacting the double stranded DNA molecule with a lipid, wherein the lipid is linked to a targeting moiety that binds a T cell antigen;
thereby making the tLNP composition.
   195. A method of making a tLNP composition, the method comprising:
(a) providing a lipid covalently linked to a first click handle;
(b) providing a targeting moiety that binds a T cell antigen, wherein the targeting moiety is covalently linked to a second click handle;
(c) contacting (a) with (b) under conditions that allow for reaction of the first click handle with the second click handle, thereby producing a click linker between the lipid and the targeting moiety;
(d) contacting the lipid and the targeting moiety with a double stranded DNA molecule; thereby making the tLNP composition.
   196. A method of making a tLNP composition, the method comprising:
(a) providing a lipid covalently linked to a first click handle;
(b) contacting the lipid with a double stranded DNA molecule, thereby forming an LNP composition;
(c) contacting the LNP composition with a targeting moiety that binds a T cell antigen, wherein the targeting moiety is covalently linked to a second click handle, under conditions that allow for reaction of the first click handle with the second click handle, thereby producing a click linker between the lipid and the targeting moiety;
thereby making the tLNP composition.
   197. The method of any of embodiments 194-196, wherein the tLNP composition has a nitrogen:phosphate (N/P) ratio of 3-9, e.g., 4-8 or 5-7, e.g., about 6.
   198. The method of any of embodiments 194-197, wherein the double stranded DNA molecule comprises a sense strand and an antisense strand, and wherein the sense strand comprises one or more chemically modified nucleobases, and wherein the antisense strand does not comprise any chemically modified nucleobases.
   199. The method of any of embodiments 194-198, wherein the tLNP composition is the tLNP composition of any of embodiments 1-136 or 146-151.
   200. The method of any of embodiments 194-199, wherein the tLNP composition further comprises an RNA molecule comprising a second effector sequence that encodes a second effector.
   201. A tLNP composition made by the method of any of embodiments 194-200.
   202. A method of making or preparing a pharmaceutical composition, the method comprising:
(a) providing a test batch comprising an LNP, a test targeting moiety that binds a T cell antigen on the surface of the LNP, and a double stranded DNA molecule comprising an effector sequence that encodes an effector;
(b) measuring or having measured one or more of:
   (i) a level of binding to a T cell antigen, e.g., CD3, in a sample of the test batch;
   (ii) a level of binding to a T cell in a sample of the test batch;
   (iii) a molar ratio of the test targeting moiety to the LNP in a sample of the test batch;
   (iv) a molar ratio of the test targeting moiety to a selected lipid in the LNP in a sample of the test batch;
   (v) expression of the effector in a cell having the T cell antigen, wherein the cell has been contacted with a sample of the test batch; or
   (vi) potency of a sample of the test batch, e.g., when contacted with a cell having the T cell antigen; and
(c) if the measurement of (b) is equal to or greater than a pre-determined threshold, or if the measurement of (b) falls within a pre-determined range, then performing or having performed one or more of:
   (i) formulating or having formulated the test batch as a pharmaceutical composition;
   (ii) dividing the test batch or pharmaceutical composition into a plurality of portions;
   (iii) contacting the test batch or pharmaceutical composition, or a portion thereof, with an excipient;
   (iv) placing the test batch or pharmaceutical composition, or a portion thereof, in a container;
   (v) labeling a container comprising the test batch or pharmaceutical composition, or a portion thereof;
   (vi) distributing the test batch or pharmaceutical composition, or a portion thereof;
   (vii) storing the test batch or pharmaceutical composition, or a portion thereof;
   (viii) releasing the test batch or pharmaceutical composition, or a portion thereof, into commerce; or
   (ix) performing a pharmaceutical release specification.
      203. The method of embodiment 202, wherein the DNA molecule comprises a first strand (e.g., sense strand) and a second strand (e.g., antisense strand), wherein the first strand (e.g., sense strand) of the DNA molecule comprises one or more chemically modified nucleobases, and wherein the second strand (e.g., antisense strand) is substantially free of (e.g., is free of) chemically modified nucleobases (e.g., a double stranded DNA molecule described herein).
      204. The method of embodiment 202 or 203, wherein step (a) comprises contacting the DNA molecule with a lipid, wherein the lipid is linked to the test targeting moiety, under conditions that allow for formation of the LNP, thereby preparing a test batch.
      205. The method of embodiment 202 or 203, wherein step (a) comprises conjugating or having conjugated a lipid to the test targeting moiety, e.g., via a click linker, thereby forming a conjugated lipid composition; and contacting the conjugated lipid composition with the DNA molecule, under conditions that allow for formation of the LNP, thereby producing a test batch.
      206. The method of embodiment 202 or 203, wherein step (a) comprises:
providing a lipid, wherein the lipid is covalently linked to a first click handle;
contacting the lipid with the DNA molecule, thereby forming a LNP composition;
contacting the LNP composition with the test targeting moiety, wherein the test targeting moiety is covalently linked to a second click handle, under conditions that allow for reaction of the first click handle with the second click handle, thereby producing a click linker between the lipid and the test targeting moiety;
thereby producing a test batch.
   207. The method of any of embodiments 202-206, wherein the test targeting moiety comprises an antibody or ligand that binds one of: CD3, CD5, CD7, CD28, 4-1BBL, CD4, CD8, CD80, CD86, CD58, CD70, SECTM1, OX40L, ICOS-L, ICAM-1, CD2, 4-1BB, or OX40.
   208. The method of any of embodiments 202-207, wherein the test targeting moiety comprises an antibody selected from a sdAb, Fab, Fab′, F(ab′)₂, Fv, or scFv.
   209. The method of any of embodiments 202-208, wherein the test batch comprises the tLNP composition of any of embodiments 1-140, 146-151, or 201.
   210. The method of any of embodiments 202-209, wherein the test batch comprises the tLNP composition made by the method of any of embodiments 194-200.
   211. The method of any of embodiments 202-209, wherein (a) comprises performing the method of any of embodiments 194-200.
   212. The method of any of embodiments 202-211, wherein the test batch further comprises an RNA molecule comprising a second effector sequence that encodes a second effector.
   213. The method of any of embodiments 154-193 or 200, wherein the LNP comprises both the DNA molecule and the RNA molecule.
   214. The method of any of embodiments 154-193, wherein the tLNP composition comprises:
(i) a first population of LNPs that comprises the DNA molecule and does not comprise the RNA molecule, and
(ii) a second population of LNPs that comprises the RNA molecule and does not comprise the DNA molecule; and
wherein the population of (i) and the population of (ii) are administered simultaneously or sequentially.
   215. The tLNP composition, pharmaceutical composition, or method of any of the preceding embodiments, wherein the DNA molecule comprises a first strand and a second strand, wherein the first strand comprises one or more chemically modified nucleobases, and wherein the second strand is substantially free of (e.g., is free of) chemically modified nucleobases.
   216. The tLNP composition, pharmaceutical composition, or method of embodiment 215, wherein the first strand is a sense strand and the second strand is an antisense strand.
   217. The tLNP composition, pharmaceutical composition, or method of embodiment 215, wherein the first strand is an antisense strand and the second strand is a sense strand.
   218. The tLNP composition, pharmaceutical composition, or method of any of embodiments 215-217, wherein at least 20% of positions in the first strand of the DNA molecule comprise chemically modified nucleobases.
   219. The tLNP composition, pharmaceutical composition, or method of any of embodiments 215-218, wherein at least 90% of thymine or uracil positions in the first strand of the DNA molecule comprise a uracil nucleobase.

### Definitions

As used herein, the term “5′ untranslated region” (5′ UTR) refers to a region of an mRNA or pre-mRNA that is transcribed but not translated, and is 5′ of the coding region. Similarly, the term “3′ untranslated region” (3′ UTR) refers to a region of an mRNA or pre-mRNA that is transcribed but not translated, and is 3′ of the coding region.

As used herein, the term “antigen binding domain” refers to a portion of an antibody or a chimeric antigen receptor which binds an antigen. In some embodiments, an antigen binding domain binds to a cell surface antigen of a cell. In some embodiments, an antigen binding domain is or comprises an antibody. In some embodiments, an antigen binding domain is or comprises an scFv or Fab.

As used herein, the term “antibody” refers to a molecule that specifically binds to, or is immunologically reactive with, a particular antigen and includes at least the variable domain of a heavy chain of an immunoglobulin. An antibody often also includes the variable domain of a light chain of an immunoglobulin. In some embodiments, the antibody is a full-length antibody, and in other embodiments, the antibody is a fragment of a full-length antibody. Antibodies include, but are not limited to, polyclonal, monoclonal, multispecific, human, humanized, primatized, or chimeric antibodies, heteroconjugate antibodies (e.g., bi-tri- and quad-specific antibodies, diabodies, triabodies, and tetrabodies), single-domain antibodies (sdAb), epitope-binding fragments, e.g., Fab, Fab′ and F(ab′)₂, Fd, Fvs, single-chain Fvs (scFv), rlgG, single-chain antibodies, disulfide-linked Fvs (sdFv), nanobody, fragments including either a VL or VH domain, fragments produced by an Fab expression library, and anti-idiotypic (anti-Id) antibodies. Antibodies described herein can be of any type (e.g., IgG, IgE, IgM, IgD, IgA, and IgY), class (e.g., IgG1, IgG2, IgG3, IgG4, IgA1 and IgA2) or subclass of immunoglobulin molecule.

As used herein, the term “backbone modification” refers to a chemical modification to the backbone of a DNA molecule. In some embodiments, the backbone modification is a chemical modification to a phosphate group, e.g., phosphorothioate. In some embodiments, the backbone modification is a chemical modification to deoxyribose.

As used herein, the term “cancer” refers to a disease characterized by the uncontrolled growth of aberrant cells. The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors.

As used herein, the term “carrier” means a compound, composition, reagent, or molecule that facilitates or promotes the transport or delivery of a composition (e.g., a tLNP composition or a DNA molecule described herein) into a cell. For example, a carrier may be a partially or completely encapsulating agent.

As used herein, the term “chemically modified nucleotide,” as used herein with respect to DNAs, refers to a nucleotide comprising one or more structural differences relative to the canonical deoxyribonucleotides (i.e., G, T, C, and A). A chemically modified nucleotide may have (relative to a canonical nucleotide) a chemically modified nucleobase, a chemically modified sugar, a chemically modified phosphodiester linkage, or a combination thereof. No particular process of making is implied; for instance, a chemically modified nucleotide can be produced directly by chemical synthesis, or by covalently modifying a canonical nucleotide.

As used herein, the term “chemically modified nucleobase” as used herein with respect to DNAs, refers to a nucleobase comprising one or more structural differences relative to the canonical nucleobases (i.e., guanine, thymine, cytosine, and adenine). No particular process of making is implied; for instance, a chemically modified nucleobase can be produced directly by chemical synthesis, or by covalently modifying a canonical nucleobase. A canonical uracil present in DNA is considered a chemically modified nucleobase under this definition.

As used herein, the term “chemically modified cytosine nucleobase,” as used herein with respect to DNAs, refers to a chemically modified nucleobase wherein the nucleobase comprises a monocyclic 6-member ring in which carbon 4 is covalently bound to a nitrogen that is not one of the six members of the ring, wherein the nucleobase comprises one or more structural differences relative to canonical cytosine nucleobase. In some embodiments, the C-5 position of the nucleobase can have a substitution other than H. For example, the C-5 position of the nucleobase can have a substitution of —OH; -aldehyde; -carboxylic acid; -alkyl; —(CH₂)ₘOR₃, m=1-3 and R₃═H or a sugar molecule; or -propargylamino. No particular process of making is implied.

As used herein, the term “chemically modified uracil nucleobase” as used herein with respect to DNAs, refers to a chemically modified nucleobase wherein the nucleobase comprises a monocyclic 6-member ring in which carbon 4 is covalently bound to an oxygen through a double bond, and wherein the nucleobase comprises one or more structural differences relative to canonical uracil and thymine nucleobases. In some embodiments, the C-5 position of the nucleobase can have a substitution other than H or a methyl group. For example, the C-5 position of the nucleobase can have a substitution of CH₂OH; -aminoallyl; -propargylamino; or -dihydroxypentyl. No particular process of making is implied.

As used herein, the term “uracil nucleobase” encompasses both canonical uracil nucleobases and chemically modified uracil nucleobases.

As used herein the term “circular” in reference to a double-stranded DNA (dsDNA) molecule described herein, means a dsDNA molecule that lacks a free end. A circular dsDNA molecule may be covalently closed. The term circular does not imply that the DNA would appear as a perfect geometric circle under a microscope; for instance, a circular dsDNA molecule may be supercoiled. In some embodiments, the circular dsDNA molecule comprises a first strand that is circular and lacks a free end, and a second strand that is circular and lacks a free end, and the first strand and second strand hybridize with each other.

As used herein, the term “free end” in reference to a DNA molecule described herein, refers to an end of a DNA strand where the terminal nucleotide is covalently bound to exactly one other nucleotide.

As used herein, the term “chimeric antigen receptor” or “CAR” refers to a non-naturally occurring polypeptide which, when in an immune effector cell, provides the cell with specificity for a target cell and generates an intracellular signal. In some embodiments, a CAR comprises an extracellular antigen binding domain, a transmembrane domain, and a primary signaling domain which is cytoplasmic. The CAR optionally further comprises one or more costimulatory domains which are cytoplasmic.

A “click handle,” as that term is used herein, refers to a chemical moiety that is capable of reacting with a second click handle in a click reaction to produce a click linker.

A “click linker,” as that term is used herein, refers to a plurality of atoms disposed between and covalently linking entity A and entity B, wherein the click linker is formed as the product of a click reaction that links entity A and entity B. In some embodiments, the click linker has the structure of a click linker that is formed as the product of a click reaction that links entity A and entity B, but is not limited to a click linker made by any particular process. For example, a click linker may be formed by a click reaction, but a click linker can also be formed or provided by a process other than a click reaction. In an embodiment, the click linker is an alkyne/azide click linker, e.g., the click linker comprises a triazole.

A “click reaction”, as that term is used herein, refers to a range of reactions used to covalently link a first moiety and a second moiety, for convenient production of linked products. It typically has one or more of the following characteristics: it is fast, is specific, is high-yield, is efficient, is spontaneous, does not significantly alter biocompatibility of the linked entities, has a high reaction rate, produces a stable product, favors production of a single reaction product, has high atom economy, is chemoselective, is modular, is stereoselective, is insensitive to oxygen, is insensitive to water, is high purity, generates only inoffensive or relatively non-toxic byproducts that can be removed by nonchromatographic methods (e.g., crystallization or distillation), needs no solvent or can be performed in a solvent that is benign or physiologically compatible, e.g., water, stable under physiological conditions. Examples include an alkyne/azide reaction, a diene/dienophile reaction, or a thiol/alkene reaction. Other reactions can be used. In some embodiments, the click reaction is fast, specific, and high-yield. For instance, in embodiments, a fast click reaction has a second order forward rate constant of 10-200 M-1s-1, 1-20 M-1s-1, or at least 1, 2, 3, 5, 10, 20, 50, 60, 100, 200, 500, 1E3, 2E3, 5E3, 1E4, 2E4, 5E4, 1E5, 2E5, 5E5, or 1E6 M-1s-1, e.g., at 20° C. in PBS. In some embodiments, a high-yield click reaction is one which has at least 75%, at least 80%, at least 85%, at least 90%, at least 95%, at least 96%, at least 97%, at least 98%, or at least 99% yield, e.g., for a reaction time of 1 hour at 20° C. in PBS.

As used herein, the term “closed end” refers to a portion of a DNA molecule positioned at one end of a double-stranded region, in which all nucleotides within the portion of the DNA molecule are covalently attached to adjacent nucleotides on either side. A closed end may, in some embodiments, include a loop comprising one or more nucleotides that are not hybridized to another nucleotide. In some embodiments (involving “no-loop” ends), the DNA end form is simply a covalent bond between the 5′ most nucleotide of the sense strand and the 3′ most nucleotide of the antisense strand, in the case of an upstream closed end, or the 3′ most nucleotide of the sense strand and the 5′ most nucleotide of the antisense strand, in the case of a downstream closed end. In some embodiments, a dsDNA molecule comprises a first closed end (e.g., upstream of a heterologous object sequence) and a second closed end (e.g., downstream of a heterologous object sequence).

As used herein, the term “open end” refers to a portion of a DNA molecule positioned at one end of a double-stranded region, in which at least one nucleotide (a “terminal nucleotide”) is covalently attached to exactly one other nucleotide. In some embodiments, the terminal nucleotide comprises a free 5′ phosphate. In some embodiments, the terminal nucleotide comprises a free 3′ OH. In some embodiments, in a double-stranded DNA molecule comprising a first DNA strand and a second DNA strand, the open end comprises a first terminal nucleotide on the first DNA strand and a second terminal nucleotide on the second DNA strand. In some embodiments, a DNA molecule comprises a first open end (e.g., upstream of an effector sequence) and a second open end (e.g., downstream of an effector sequence). In some embodiments, the open end comprises a blunt end, a sticky end, or a Y-adaptor.

As used herein, the term “costimulatory signaling protein” refers to a surface polypeptide on a T cell that specifically binds with a costimulatory ligand, thereby mediating a costimulatory response by the T cell, such as proliferation. In some embodiments, the surface polypeptide may be a transmembrane protein having an extracellular domain. In some embodiments, a costimulatory signaling protein is CD28. In some embodiments, a targeting moiety that binds a costimulatory signaling protein comprises an extracellular domain of a costimulatory ligand (e.g., CD80, CD86, CD58, 4-1BBL (CD137L), CD70, SECTM1, OX40L, ICOS-L, or ICAM-1).

As used herein, the term “DNA molecule” refers to any compound and/or substance that comprises at least two (e.g., at least 10, at least 20, at least 50, at least 100) covalently linked deoxyribonucleotides. In some embodiments, the DNA molecule is a single oligonucleotide chain, while in other embodiments, the DNA molecule comprises a plurality of oligonucleotide chains, while in yet other embodiments the DNA molecule is a portion of an oligonucleotide chain. In some embodiments, DNA molecule is a compound and/or substance that is or can be incorporated into an oligonucleotide chain via a phosphodiester linkage. In some embodiments, the DNA molecule comprises solely canonical nucleotides. In some embodiments, the DNA molecule comprises one or more chemically modified nucleotides. In some embodiments, at least 80%, at least 85%, at least 90%, at least 95%, at least 98%, at least 99%, or 100% of the sugars of the DNA molecule are deoxyribose sugars. In some embodiments, the DNA molecule was prepared by one or more of: isolation from a natural source, enzymatic synthesis by polymerization based on a complementary template (in vivo or in vitro), reproduction in a recombinant cell or system, and chemical synthesis.

As used herein, the term “DNA end form” refers to a structure comprising DNA that is situated at an end of a double-stranded DNA molecule. In some embodiments, the DNA end form comprises a closed end. In other embodiments, the DNA end form comprises an open end. In some embodiments, the DNA end form comprises a loop, a Y-adaptor, a blunt end, or a sticky end. The DNA end form may comprise one or both of a single stranded region and a double stranded region. The DNA end form may comprise canonical nucleotides, chemically modified nucleotides, or a combination thereof. In some embodiments, the DNA end form comprises between 3-100 nucleotides. In some embodiments, the double-stranded DNA molecule comprises a first DNA end form at a first end and a second DNA end form at a second end. In some embodiments, the first DNA end form and the second DNA end form of a DNA molecule are the same type. In some embodiments, the first DNA end form and the second DNA end form of a DNA molecule are different types.

As used herein, the term “effector sequence” refers to the part of a DNA molecule that exerts a function on a cell, either directly (wherein the effector sequence is a functional DNA sequence) or by encoding a functional RNA or protein. The encoded functional RNA or protein is referred to as the “effector”.

As used herein, the term “exonuclease-resistant”, when used to describe a DNA molecule, means that the DNA molecule, if it comprises closed ends, is resistant to the exonuclease assay as described in Example 10 or Example 11 of WO/2023/220729.

As used herein, the term “heterologous”, when used to describe a first element in reference to a second element means that the first element and second element do not exist in nature disposed as described. For example, a heterologous polypeptide, nucleic acid molecule, construct or sequence refers to (a) a polypeptide, nucleic acid molecule or portion of a polypeptide or nucleic acid molecule sequence that is not native to a cell in which it is expressed, (b) a polypeptide or nucleic acid molecule or portion of a polypeptide or nucleic acid molecule that has been altered or mutated relative to its native state, or (c) a polypeptide or nucleic acid molecule with an altered expression as compared to the native expression levels under similar conditions. For example, a heterologous regulatory sequence (e.g., promoter sequence or enhancer sequence) may be used to regulate expression of a gene or a nucleic acid molecule in a way that is different than the gene or a nucleic acid molecule is normally expressed in nature. In another example, a heterologous domain of a polypeptide or nucleic acid sequence (e.g., a DNA binding domain of a polypeptide or nucleic acid encoding a DNA binding domain of a polypeptide) may be disposed relative to other domains or may be a different sequence or from a different source, relative to other domains or portions of a polypeptide or its encoding nucleic acid. In certain embodiments, a heterologous nucleic acid molecule may exist in a native host cell genome, but may have an altered expression level or have a different sequence or both. In other embodiments, heterologous nucleic acid molecules may not be endogenous to a host cell or host genome but instead may have been introduced into a host cell by transformation (e.g., transfection, electroporation), wherein the added molecule may integrate into the host genome or can exist as extra-chromosomal genetic material either transiently (e.g., mRNA) or semi-stably for more than one generation (e.g., episomal viral vector, plasmid or other self-replicating vector).

As used herein, the term “heterologous functional sequence” refers to a nucleic acid sequence that is heterologous to an adjacent (e.g., directly adjacent) nucleic acid sequence and has one or more biological function.

As used herein, the terms “increasing” and “decreasing” refer to modulating resulting in, respectively, greater or lesser amounts, of function, expression, or activity of a metric relative to a reference. For example, subsequent to administration of a DNA molecule in a method described herein, the amount of the metric described herein (e.g., the level of gene expression, or a marker of innate immunity) may be increased or decreased in a subject by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 98% or more relative to the amount of the marker prior to administration, or relative to administration of a control DNA molecule. Generally, the metric is measured subsequent to administration at a time that the administration has had the recited effect, e.g., one day, one week, one month, 3 months, or 6 months, after a treatment regimen has begun.

As used herein, the term “ligand” refers to a polypeptide or portion of a polypeptide that specifically binds to a feature of a target cell. In some embodiments, the ligand comprises the amino acid sequence of a naturally occurring protein, or a fragment thereof, that binds to a T cell surface marker. In some embodiments, the ligand comprises an amino acid sequence that is at least 85%, at least 90%, or at least 95% identical to the amino acid sequence of a naturally occurring protein, or a fragment thereof.

As used herein, the term “linear” in reference to a double-stranded DNA molecule described herein, means a nucleic acid comprising two DNA strands or portions of strands which hybridize with each other (thereby forming a double stranded region), wherein the structure comprises two ends. An end may be a closed end or an open end. The two strands that hybridize with each other may be partially or completely complementary. In some embodiments, a linear double-stranded DNA molecule consists of a single strand of DNA that, under denaturing conditions, does not form any double stranded regions and does not have any free ends, and wherein under physiological conditions a first portion of the strand hybridizes to a second portion of the strand (thereby forming a double stranded region), and the linear double-stranded DNA molecule comprises a first closed end comprising a first loop and a second closed end comprising a second loop.

As used herein, when two entities are “linked”, the two entities are physically connected by means of one or more covalent or noncovalent bond. In some embodiments, the two entities are directly linked, i.e., an atom of the first entity forms a covalent or noncovalent bond with an atom of the second entity. In some embodiments, the two entities are indirectly linked through a third entity; for example A is linked to C by virtue of A being directly linked to B and B being directly linked to C.

As used herein, the terms “linker” refers to a moiety that connects two parts of a compound, e.g., covalently attaches two parts of a compound. The linker may be a click linker.

As used herein, the term “loop” refers to a nucleic acid sequence that is single stranded. A loop is connected at both ends by a double stranded region referred to as a “stem”, to form a “stem-loop”.

As used herein, the term “maintenance sequence” is a DNA sequence or motif that enables or facilitates retention of a DNA molecule in the nucleus through cell division. A maintenance sequence typically enables replication and/or transcription of DNA in the nucleus by interacting with proteins that facilitate chromatin looping. An example of a maintenance sequence is a scaffold/matrix attached region (S/MAR element).

As used herein, a “nuclear targeting sequence” is a DNA sequence that enables or facilitates DNA entry into a target cell nucleus.

As used herein, a “pharmaceutical composition” or “pharmaceutical preparation” is a composition or preparation which is indicated for animal, e.g., human or veterinary pharmaceutical use, for example, non-human animal or human prophylactic or therapeutic use. A pharmaceutical preparation comprises an active agent having a biological effect on a cell or tissue of a subject, e.g., having pharmacological activity or an effect in the mitigation, treatment, or prevention of disease, in combination with a pharmaceutically acceptable excipient or diluent. A pharmaceutical composition also means a finished dosage form or formulation of a prophylactic or therapeutic composition.

As used herein, the terms “peptide,” “polypeptide,” and “protein” are used interchangeably and refer to a compound comprising amino acid residues covalently linked by peptide bonds, or by means other than peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds or by means other than peptide bonds. As used herein, the term refers to both short chains, which also commonly are referred to in the art as peptides, oligopeptides and oligomers, for example, and to longer chains, which generally are referred to in the art as proteins, of which there are many types. In some embodiments, a polypeptide comprises a non-canonical amino acid residue.

As used herein, the term “polyadenylation site” refers to a segment of an mRNA or pre-mRNA that is cleaved and polyadenylated. A polyadenylation site typically comprises the sequence CA.

As used herein, the term “polyadenylation signal” refers to a segment of an mRNA or pre-mRNA that recruits polyadenylation machinery. A polyadenylation signal typically comprises the sequence AAUAAA.

As used herein, a “sense strand” of a DNA molecule is a strand which has the same sequence as an mRNA or pre-mRNA which encodes for a functional RNA or protein, and does not serve as a template for transcription. An “antisense strand” of a DNA molecule is a strand that has a sequence complementary to an mRNA or pre-mRNA which encodes for a functional RNA or protein and/or can serve as a template for transcription.

As used herein, the term “double-stranded DNA molecule” or dsDNA molecule means a DNA composition comprising two complementary chains of deoxyribonucleotides that base pair to each other. The two complementary strands may have perfect complementarity or may have one or more mismatches, e.g., forming bulges. Either of the two strands may, in some embodiments, have paired regions of self-complementarity that form intramolecular/intrastrand double stranded motifs in a folded configuration, for example, may form hairpin loops, junctions, bulges or internal loops. In some embodiments, the dsDNA molecule comprises one or two closed ends. In some embodiments, the dsDNA molecule is circular or linear. In some embodiments (e.g., in a dsDNA molecule with closed ends) the two complementary chains of deoxyribonucleotides are covalently linked.

As used herein, the term “signaling domain” refers to a functional portion of a protein which acts by transmitting information within the cell to regulate cellular activity via one or more defined signaling pathways. In some embodiments, a signaling domain is intracellular, e.g., is an intracellular domain of a protein that also comprises a transmembrane domain. In some embodiments, a signaling domain can generate a signal that promotes an immune effector function of a CAR-containing cell.

As used herein, the term “T cell antigen” refers to an antigen on the surface of a T cell. In some embodiments, the T cell antigen is a protein. In some embodiments, the T cell antigen comprises a receptor, or a fragment thereof. In some embodiments, the T cell antigen comprises a costimulatory signaling protein, or a fragment thereof. A T cell antigen need not be uniquely present on T cells; it may also be present on a cell other than a T cell.

As used herein, the term “targeting moiety” is an agent which associates or interacts with a feature of a target cell which may be used to specifically (relative to at least one other cell in the relevant system) target an LNP to the cell. In some embodiments, the targeting moiety specifically binds to a feature of a target cell. In some embodiments, the targeting moiety is a domain of a polypeptide, and the feature of the target cell is a cell surface protein.

As used herein, the term “therapeutic double stranded construct” (“TDSC”) refers to a linear construct comprising DNA, wherein the construct is at least partially double stranded. A TDSC does not comprise a plasmid backbone sequence (e.g., does not comprise a bacterial origin of replication). A TDSC does not comprise a viral capsid or a viral envelope. In some embodiments, the TDSC comprises a closed end or an open end (e.g., a blunt end or a sticky end). In some embodiments, the TDSC is suitable for administration to a human subject.

As used herein, the term “terminal nucleotide” refers to a nucleotide that is covalently attached to exactly one other nucleotide. In some embodiments, the terminal nucleotide comprises a free 5′ phosphate. In some embodiments, the terminal nucleotide comprises a free 3′ OH.

As used herein, “treatment” and “treating” refer to the medical management of a subject with the intent to improve, ameliorate, stabilize (i.e., not worsen), prevent or cure a disease, pathological condition, or disorder. This term includes active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to minimizing or partially or completely inhibiting the development of the associated disease, pathological condition, or disorder); and supportive treatment (treatment employed to supplement another therapy). Treatment also includes diminishment of the extent of the disease or condition; preventing spread of the disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to have the condition or disorder or those in which the condition or disorder is to be prevented.

As used herein, the term “Y-adaptor” refers to a nucleic acid structure comprising a first nucleic acid region and a second nucleic acid region which are complementary (e.g., perfectly complementary) to each other; the first and second regions may hybridize to form a double stranded region. The first nucleic acid region is covalently linked to a third nucleic acid region, and the second nucleic acid region is covalently linked to a fourth nucleic acid region, and the third and fourth nucleic acid regions are not substantially complementary to each other; the third and fourth regions may be single stranded. The first nucleic acid region is 3′ of the third nucleic acid region and the second nucleic acid region is 5′ of the fourth nucleic acid region. As a result, the third and fourth regions may be situated on the same side of the double stranded regions. The Y-adaptor may be part of a double-stranded DNA molecule.

### BRIEF DESCRIPTION OF THE DRAWINGS

FIG. 1 is a graph depicting the percentage of GFP-positive cells in pre-activated T cells transfected with tLNPs comprising circular dsDNA molecules lacking chemically modified nucleobases (“cheDNA-unmod”), or circular “hemi-modified” dsDNA molecules comprising either 5-hydroxymethyluracil (“cheDNA-hmU”) or canonical uracil (“cheDNA-dU”) on the sense strand. The tLNPs were contacted with the pre-activated T cells at a final concentration of 2 μg of DNA/mL of media (“tLNP low”), 8 μg of DNA/mL of media (“tLNP med”), or 16 μg of DNA/mL of media (“tLNP high”). Data is shown for primary T cells from three different donors.

FIG. 2 is a graph depicting the percentage of GFP-positive cells in naïve T cells transfected with tLNPs comprising circular dsDNA molecules lacking chemically modified nucleobases (“cheDNA-unmod”), circular “hemi-modified” dsDNA molecules comprising canonical uracil on the sense strand (“cheDNA-dU”), or PBS control. The percentage of GFP-positive cells was measured at Day 2 and Day 4 following transfection. Data is shown for primary T cells from three different donors.

FIG. 3 is a graph depicting the level of 2′-3′ cGAMP (pmol/mL) in pre-activated T cells following transfection with tLNPs comprising either mRNA, circular dsDNA molecules lacking chemically modified nucleobases (“Unmodified”), or circular “hemi-modified” dsDNA molecules with either 5-hydroxymethyluracil (“Mod 1”) or canonical uracil (“Mod 2”) on the sense strand. The tLNPs were contacted with the T cells at a final concentration of 8 μg of RNA or DNA per mL of media. Data is shown for primary T cells from three different donors.

### DETAILED DESCRIPTION

This disclosure relates to compositions and methods for providing an effector, e.g., a therapeutic effector, to a cell, tissue or subject, e.g., in vivo or in vitro. The effector may be a DNA sequence, a polypeptide, e.g., a therapeutic protein, or an RNA, e.g., a regulatory RNA or an mRNA.

In some aspects, the present disclosure provides a targeted lipid nanoparticle (tLNP) composition comprising: a) a lipid nanoparticle (LNP), b) a targeting moiety on the surface of the LNP, wherein the targeting moiety binds a T cell antigen, and c) a double stranded DNA molecule comprising an effector sequence that encodes an effector, wherein the DNA molecule comprises a first strand and a second strand, wherein the first strand comprises one or more chemically modified nucleobases and the second strand is free of chemically modified nucleobases.

### Elements of DNA Molecules

The DNA molecules described herein can contain elements sufficient to deliver an effector sequence to a target cell, tissue or subject. In some embodiments, the effector sequence is a DNA sequence. In some embodiments, the DNA molecule drives expression of an effector, e.g., the DNA molecule comprises a promoter sequence and a sequence encoding an RNA or a polypeptide, e.g., a therapeutic RNA or polypeptide. In some embodiments, the DNA molecules described herein further contain a maintenance sequence.

In some embodiments, the DNA molecule comprises a double-stranded DNA molecule. In some embodiments, the DNA molecule comprises a circular double-stranded DNA molecule. In some embodiments, the circular double-stranded DNA molecule comprises a plasmid or a minicircle. In some embodiments, the DNA molecule comprises a linear double-stranded DNA molecule (e.g., TDSC).

In some embodiments, a DNA molecule disclosed herein is at least about 10 nucleotides, at least about 15 nucleotides, at least about 20 nucleotides, at least about 25 nucleotides, at least about 30 nucleotides, at least about 40 nucleotides, at least about 50 nucleotides, at least about 60 nucleotides, at least about 70 nucleotides, at least about 75 nucleotides, at least about 80 nucleotides, at least about 90 nucleotides, at least about 100 nucleotides, at least about 200 nucleotides, at least about 300 nucleotides, at least about 400 nucleotides, at least about 500 nucleotides, at least about 600 nucleotides, at least about 700 nucleotides, at least about 800 nucleotides, at least about 900 nucleotides, at least about 1000 nucleotides, at least about 2000 nucleotides, at least about 3000 nucleotides, at least about 4000 nucleotides, at least about 5000 nucleotides, at least about 6000 nucleotides, at least about 7000 nucleotides, at least about 8000 nucleotides, at least about 9000 nucleotides, at least about 10,000 nucleotides, at least about 11,000, or at least about 12,000 nucleotides in length. In some embodiments, a DNA molecule disclosed herein is less than 50, less than 60, less than 70, less than 80, less than 90, less than 100, less than 200, less than 300, less than 400, less than 500, less than 600, less than 700, less than 800, less than 900, less than 1000, less than 2000, less than 3000, less than 4000, less than 5000, less than 6000, less than 7000, less than 8000, less than 9000, less than 10,000, less than 11,000, or less than 12,000 nucleotides in length. In some embodiments, a DNA molecule disclosed herein is between 10-15, 15-20, 20-25, 25-30, 30-35, 35-40, 40-45, 45-50, 50-55, 55-60, 60-70, 70-80, 80-90, 90-100, 20-30, 30-40, 40-50, 50-75, 75-100, 100-200, 200-300, 300-400, 400-500, 300-500, 500-600, 600-700, 700-800, 800-900, 900-1000, 500-1000, 1000-2000, 2000-3000, 3000-4000, 4000-5000, 5000-6000, 6000-7000, 7000-8000, 8000-9000, 9000-10,000, 10,000-11,000, or 11,000-12,000 nucleotides in length. In some embodiments, the size of a DNA molecule disclosed herein is a length sufficient to encode useful polypeptides or RNAs.

In some embodiments, a DNA molecule described herein is resistant to endonuclease digestion and/or resistant to immune sensor recognition. In some embodiments, at least 80%, at least 85%, at least 90%, at least 95%, at least 98%, at least 99%, or 100% of the sugars of a DNA molecule described herein are deoxyribose sugars.

In some embodiments, a DNA molecule described herein can be replicated (e.g., by a DNA polymerase native to a cell comprising the DNA molecule). In some embodiments, a DNA molecule described herein cannot be replicated. In some embodiments, a DNA molecule or a portion thereof can be integrated into the genome. In some embodiments, a DNA molecule or a portion thereof cannot be integrated into the genome.

In some embodiments, a double-stranded DNA molecule comprises an exonuclease-resistant DNA end form (e.g., as described herein). In some embodiments, the DNA end form is at least 2, at least 3, at least 4, at least 5, at least 10, at least 15, at least 20, at least 25, at least 30, at least 40, at least 50, at least 60, at least 70, at least 80, at least 90, or at least 100 nucleotides in length. In some embodiments, the DNA end form is less than 10, less than 15, less than 20, less than 25, less than 30, less than 40, less than 50, less than 60, less than 70, less than 80, less than 90, or less than 100 nucleotides in length. In some embodiments, the DNA end form is 2-5, 5-10, 10-15, 15-20, 20-25, 25-30, 30-35, 35-40, 40-45, 45-50, 50-55, 55-60, 60-70, 70-80, 80-90, or 90-100 nucleotides in length. It is understood that when the length of a linear closed-ended dsDNA molecule is discussed herein, the length refers to the number of nucleotides starting with and including the upstream end, through the downstream end. For example, a no-loop dsDNA molecule having 100 base pairs would have a length of 100 nucleotides.

In some embodiments, a double-stranded DNA molecule comprises an effector sequence encoding an effector (e.g., a polypeptide or RNA, e.g., as described herein), e.g., positioned between two exonuclease-resistant DNA end forms.

A double-stranded DNA molecule described herein may have less than a threshold level of single stranded structures. In one embodiment, the double-stranded DNA molecule does not comprise more than 20, 18, 16, 14, 12, 10, 8, 7, 5, 4, 3, 2, or 1 single stranded region longer than 100, 80, 70, 60, 50, 40, 30, 20 or 10 bases, e.g., does not comprise single stranded regions longer than 100, 80, 70, 60, 50, 40, 30, 20 or 10 bases.

In some embodiments, a double-stranded DNA molecule comprises a sense strand and an antisense strand.

In some embodiments, a DNA molecule comprises (a) an upstream end form (e.g., upstream exonuclease-resistant DNA end form); (b) double-stranded DNA; and (c) a downstream end form (e.g., exonuclease-resistant DNA end form). In some embodiments, the upstream exonuclease-resistant DNA end form has a loop size of less than about 28 or 56 nucleotides in length or greater than about 28 or 56 nucleotides in length. In some embodiments, the downstream exonuclease-resistant DNA end form has a loop size of less than about 28 or 56 nucleotides in length or greater than about 28 or 56 nucleotides in length. In some embodiments, every nucleotide in the DNA molecule binds another nucleotide in the DNA molecule.

In some embodiments, the upstream DNA end form and the downstream DNA end form have the same nucleotide sequence. In some embodiments, the upstream DNA end form and the downstream DNA end form have different nucleotide sequences. In some embodiments, the upstream exonuclease-resistant DNA end form and the downstream exonuclease-resistant DNA end form have the same structure. In some embodiments, the upstream exonuclease-resistant DNA end form and the downstream exonuclease-resistant DNA end form have different structures.

In some embodiments, a double-stranded DNA molecule described herein is linear and can be circularized. In some embodiments, a double-stranded DNA molecule described herein is linear and cannot be circularized. In some embodiments, a double-stranded DNA molecule described herein can be concatemerized. In some embodiments, a double-stranded DNA molecule described herein cannot be concatemerized.

In some embodiments, a dsDNA form described herein is asymmetrically modified, where one strand comprises chemically modified nucleobases and the other strand is substantially free of chemically modified nucleobases. In some embodiments, the hemi-modified DNA may be completely free of chemically modified nucleotides on the antisense strand, and in other embodiments, the hemi-modified DNA may comprise a few chemical modifications (such as backbone modifications, e.g., phosphorothioate) on the antisense strand. In some embodiments, the hemi-modified DNA molecule comprises chemically modified nucleotides (e.g., nucleotides comprising chemically modified nucleobases) on the sense strand.

### Exonuclease-Resistant DNA End Forms

In some embodiments, a double-stranded DNA molecule described herein comprises a DNA end form at each end of the double-stranded DNA molecule. The DNA end forms described herein can, in some instances, comprise a closed end, wherein every nucleotide of the DNA end form is covalently attached to two other nucleotides of the DNA end form. In other instances, the DNA end forms described herein comprise an open end comprising at least one nucleotide that is only covalently attached to one other nucleotide of the DNA end form. The DNA end forms are generally exonuclease resistant.

In some embodiments, a DNA molecule described herein comprises an upstream DNA end form which is a closed end; (b) a double stranded region; and (c) a downstream DNA end form which is a closed end.

Exemplary exonuclease-resistant DNA end forms, the production of exonuclease-resistant DNA end forms, and assessment of exonuclease resistance can be found, for example, in WO/2023/220729, incorporated herein by reference in its entirety.

### Loops

In some embodiments, a DNA end form comprises a loop.

The DNA end form comprising a loop may be produced, e.g., by ligating an end adaptor which is a hairpin to a dsDNA molecule. A hairpin generally comprises a single-stranded loop region covalently attached at both the 5′ and 3′ ends to a double-stranded stalk region. In certain embodiments, the single-stranded loop region comprises one or more nucleotides (e.g., 1-2, 2-5, 5-10, 10-15, 15-20, 20-25, 25-30, 30-35, or 35-40 nucleotides) that are not hybridized to another nucleotide. In embodiments, the loop is comprised in a DNA molecule having a doggybone conformation.

### Y-Adaptors

In some embodiments, an exonuclease-resistant DNA end form as described herein comprises a Y-adaptor. As described herein, a Y-adaptor generally comprises a pair of single-stranded DNA regions, each attached at one end to a strand of a double-stranded DNA region, thereby forming a “Y” shape (wherein the base of the “Y” represents the double-stranded DNA region, and each of the upper prongs of the “Y” represents the two single-stranded DNA region).

### No Loop Closed DNA End Forms

In some embodiments, a double-stranded DNA molecule as described herein comprises an exonuclease-resistant DNA end form that is covalently closed but does not include a single stranded loop. For example, in certain embodiments, every nucleotide of a covalently-closed DNA molecule is complementary to another nucleotide. Accordingly, the DNA end form may be a bond between the endmost nucleotide of the sense strand and the nucleotide of the antisense strand which base pairs with the nucleotide of the sense strand.

### Open DNA End Forms

In some embodiments, a double-stranded DNA molecule as described herein comprises an exonuclease-resistant DNA end form that is not covalently closed. In certain embodiments, the DNA end form comprises a blunt end (e.g., a blunt end comprising one or more chemical modifications as described herein) or a sticky end (e.g., a sticky end comprising one or more chemical modifications as described herein).

### Inverted Terminal Repeats (ITRs)

In some embodiments, a double-stranded DNA molecule as described herein comprises an exonuclease-resistant DNA end form comprising an inverted terminal repeat (ITR). In some embodiments, the ITR is an ITR from a virus, e.g., an adenovirus or an adeno-associated virus (AAV). In some embodiments, the ITR comprises a nucleic acid sequence having at least 75%, at least 80%, at least 85%, at least 90%, at least 95%, at least 96%, at least 97%, at least 98%, at least 99%, or 100% sequence identity to an ITR sequence from a virus, e.g., an adenovirus or an adeno-associated virus (AAV). In certain embodiments, the ITR comprises an origin of replication (e.g., a viral origin of replication). In embodiments, a double-stranded DNA molecule as described herein comprises an exonuclease-resistant DNA end form comprising an ITR (e.g., as described herein) at each end. In some embodiments, a double-stranded DNA molecule does not comprise an ITR.

### Sequence Elements of DNA Molecules

In some embodiments, a DNA molecule described herein comprises a promoter sequence. In some embodiments, a DNA molecule described herein comprises an effector sequence (e.g., a therapeutic effector sequence) operably linked to the promoter sequence. In some embodiments, a DNA molecule described herein comprises a heterologous functional sequence. In some embodiments, a DNA molecule described herein comprises a maintenance sequence. In some embodiments, a DNA molecule described herein comprises an origin of replication. In some embodiments, the DNA molecule comprises one, two, three, four, or all of a promoter sequence, an effector sequence, a heterologous functional sequence, a maintenance sequence, or an origin of replication. In some embodiments, the DNA molecule comprises a promoter sequence, an effector sequence, and a heterologous functional sequence. In some embodiments, the DNA molecule comprises a promoter sequence, an effector sequence, and a maintenance sequence. In some embodiments, the DNA molecule comprises a promoter sequence, an effector sequence, and an origin of replication. In some embodiments, the DNA molecule comprises a promoter sequence, an effector sequence, a heterologous functional sequence, and a maintenance sequence. In some embodiments, the DNA molecule comprises a promoter sequence, an effector sequence, a heterologous functional sequence, and an origin of replication. In some embodiments, the DNA molecule comprises a promoter sequence, an effector sequence, a maintenance sequence, and an origin of replication. In some embodiments, the DNA molecule comprises a promoter sequence, an effector sequence, a heterologous functional sequence, a maintenance sequence, and an origin of replication.

In some embodiments, a DNA molecule described herein comprises an effector sequence that encodes an effector. In some embodiments, the effector sequence encodes a polypeptide (e.g., a protein). In some embodiments, the effector sequence encodes a functional RNA (e.g., a miRNA, siRNA, or tRNA). In some embodiments, the effector sequence is heterologous to a target cell.

### Promoter Sequences and Other Regulatory Sequences

The DNA molecule described herein may contain a promoter sequence (a DNA sequence at which RNA polymerase and transcription factors bind to, directly or indirectly, to initiate transcription) operably linked to an effector sequence. A promoter sequence may be found in nature operably linked to the effector sequence, or may be heterologous to the effector sequence. A promoter sequence described herein may be native to the target cell or tissue, or heterologous to the target cell or tissue. A promoter sequence may be constitutive, inducible and/or tissue-specific.

Examples of constitutive promoter sequences include sequences of the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985), the dihydrofolate reductase promoter, the beta-actin promoter, the phosphoglycerol kinase (PGK) promoter, and the EF1alpha promoter.

Inducible promoter sequences allow regulation of expression and can be regulated by exogenously supplied compounds, environmental factors such as temperature, or the presence of a specific physiological state, e.g., acute phase, a particular differentiation state of the cell, or in replicating cells only. Inducible promoter sequences and inducible systems are available from a variety of sources. Examples of inducible promoter sequences regulated by exogenously supplied promoters include sequences of the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al., Proc. Natl. Acad. Sci. USA, 89:5547-5551 (1992)), the tetracycline-inducible system (Gossen et al., Science, 268:1766-1769 (1995), see also Harvey et al, Curr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997)).

In some embodiments, the native promoter sequence for the sequence encoding the effector can be used.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoter sequences, enhancer sequences, etc.) are known in the art. Exemplary tissue-specific promoter sequences include, but are not limited to, sequences of: a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a alpha-myosin heavy chain (a-MHC) promoter, or a cardiac Troponin T (cTnT) promoter. Other exemplary promoter sequences include sequences of the Beta-actin promoter, hepatitis B virus core promoter, Sandig et al., Gene Ther., 3:1002-9 (1996); alpha-fetoprotein (AFP) promoter, Arbuthnot et al., Hum. Gene Ther., 7:1503-14 (1996)), bone osteocalcin promoter (Stein et al., Mol. Biol. Rep., 24:185-96 (1997)); bone sialoprotein promoter (Chen et al., J. Bone Miner. Res., 11:654-64 (1996)), CD2 promoter (Hansal et al., J. Immunol., 161:1063-8 (1998); immunoglobulin heavy chain promoter; T cell receptor alpha-chain promoter, neuronal such as neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991)), and the neuron-specific vgf gene promoter (Piccioli et al., Neuron, 15:373-84 (1995)), among others which will be known to the skilled artisan.

Examples of tissue/cell specific promoter sequences are listed in Table 1:

TABLE 1
Tissue or cell specific promoter sequences

| | | Accession Number; Human |
|---|---|---|
| Tissue/Cell | Promoter | Genome Coordinate (hg38) |
| Skeletal | ACTA1 | NM_001100; chr1: 229,439,090- |
| muscle | | 229,432,090 |
| Melanoma | TYR | NM_000372; chr11: 89,300,750- |
| | | 89,293,750 |
| Hepatoma | a- | NM_001354717; chr4: 73,461,175- |
| | fetoprotein | 73,454,175 |
| Mammary | Mucin 1 | NM_001371720; |
| carcinoma | | chr1: 155,197,900-155,190,900 |
| Prostate | KLK3 | NM_001648; chr19: 50,865,760- |
| Cancer | | 50,858,760 |
| Neuronal | ENO2 | NM_001975; chr12: 6,928,700- |
| cells | | 6,921,700 |
| Response to | HIF- | NM_001530; chr14: 61,753,200- |
| Hypoxia | 1alpha | 61,746,200 |
| Retinoblastoma | E2F1 | NM_005225; chr20: 33,691,380- |
| | | 33,684,380 |
| Ionizing | EGR-1 | NM_001964; chr5: 138,474,303- |
| radiation | | 138,467,303 |
| Oncogene | ErbB2 | NM_004448; chr17: 39,735,530- |
| | | 39,728,530 |
| Endothelial | vWF | NM_000552; chr12: 6,129,670- |
| cells | | 6,122,670 |
| Endothelial | FLT-1 | NM_002019; chr13: 28,500,100- |
| cells | | 28,493,100 |
| Endothelial | ICAM-2 | NM_001099786; |
| cells | | chr17: 64,025,630-64,018,630 |
| Retinal | VMD2 | NM_004183; chr11: 61,972,630- |
| pigment | | 61,965,630 |
| epithelium | | |
| Rod cells | RHO | NM_000539; chr3: 129,540,350- |
| | | 129,533,350 |
| Cone cells | Red/green | NM_020061; chrX: 154,164,030- |
| | opsin | 154,157,030 |
| | (OPN1LW) | |
| Ganglion | Thymocyte | NM_006288; chr11: 119,428,150- |
| cells | antigen | 119,421,150 |
| | (Thy1) | |
| T cells | TIM3 | NM_032782; chr5: 157,114,050- |
| | | 157,107,050 |
| T cells | FOXP3 | NM_014009; chrX: 49,269,700- |
| | | 49,262,700 |
| PBMCs | Vβ6.7 | ENST00000390373.2; |
| | | chr7: 142,493,295-142,486,295 |
| Cell cycle | Cdk1 | NM_001786; chr10: 60,799,850- |
| | | 60,792,850 |

The DNA molecules described herein may also include other native or heterologous expression control elements, such as enhancer elements, a sequence encoding a polyadenylation site, or Kozak consensus sequences. In some embodiments, a DNA molecule described herein comprises a sequence encoding a polyadenylation site. In some embodiments, a DNA molecule described herein comprises a sequence encoding a polyadenylation signal (e.g., bGH polyadenylation signal).

### Effector Sequence

The effector sequence of a DNA molecule described herein may be, e.g., a functional DNA sequence, e.g., a therapeutically functional DNA sequence; a DNA sequence encoding a therapeutic peptide, polypeptide or protein; or a DNA sequence encoding a therapeutic RNA (e.g., a non-coding RNA).

### DNA Effectors:

A therapeutically functional DNA sequence may be a DNA sequence that forms a functional structure, e.g., a DNA sequence comprising a DNA aptamer, DNAzyme or allele-specific oligonucleotide (a DNA ASO). A therapeutically functional DNA sequence typically lacks a promoter sequence operably linked. In some embodiments, a DNA molecule described herein may include one or a plurality of functional DNA sequences, e.g., 2, 3, 4, 5, 6, or more sequences, which may be the same or different.

### Polypeptide Effectors:

A DNA sequence encoding a therapeutic polypeptide may be a DNA sequence encoding one or more effectors which is a peptide, protein, or combinations thereof. For example, the DNA sequence encodes an mRNA. The peptide or protein may be: a DNA binding protein; an RNA binding protein; a transporter; a transcription factor; a translation factor; a ribosomal protein; a chromatin remodeling factor; an epigenetic modifying factor; an antigen; a hormone; an enzyme (such as a nuclease, e.g., an endonuclease, e.g., a nuclease element of a CRISPR system, e.g., a Cas9, dCas9, aCas9-nickase, Cpf/Cas12a); a CRISPR-linked enzyme, e.g. a base editor or prime editor; a mobile genetic element protein (e.g., a transposase, a retrotransposase, a recombinase, an integrase); a gene writer; a polymerase; a methylase; a demethylase; an acetylase; a deacetylase; a kinase; a phosphatase; a ligase; a deubiquitinase; a protease; an integrase; a recombinase; a topoisomerase; a gyrase; a helicase; a lysosomal acid hydrolase); an antibody (e.g., an intact antibody, a fragment thereof, or a nanobody); a signaling peptide; a receptor ligand; a receptor; a clotting factor; a coagulation factor; a structural protein; a caspase; a membrane protein; a mitochondrial protein; a nuclear protein; or an engineered binder such as a centyrin, darpin, or adnectin. See, e.g., Gebauer & Skerra. 2020. Annual Review of Pharmacology and Toxicology 60:1, 391-415.

In some embodiments, a DNA molecule described herein may include one or a plurality of sequences encoding a polypeptide, e.g., 2, 3, 4, 5, 6, or more sequences encoding a polypeptide. Each of the plurality may encode the same or different protein. For example, a sequence described herein may include multiple sequences encoding multiple proteins, e.g., a plurality of proteins in a biological pathway.

In some embodiments, a DNA molecule described herein may include a plurality of sequences encoding a polypeptide, e.g., 2, 3, 4, 5, 6, or more sequences encoding a polypeptide, separated by a self-cleaving peptide, e.g., P2A, T2A, E2A or F2A. Self-cleaving peptides are typically 18-22 amino acids long, and can induce ribosomal skipping during protein translation so that two polypeptides can be encoded in the same transcript. Each of the polypeptides may encode the same or different protein. In one embodiment, a DNA molecule described herein may include a promoter sequence followed by a sequence encoding a first polypeptide of interest, a sequence encoding a 2A self-cleaving peptide, a sequence encoding a second polypeptide of interest, and a sequence encoding a polyA site. In another embodiment, a DNA molecule described herein may include a promoter sequence followed by a sequence encoding the first polypeptide of interest, a first 2A self-cleaving peptide, a second polypeptide of interest, a sequence encoding a second 2A self-cleaving peptide, a sequence encoding a third polypeptide of interest, and a sequence encoding a polyA site.

In some embodiments, the effector comprises a cell penetrating polypeptide. In some embodiments, the effector is a fusion protein that comprises a cell penetrating polypeptide and a second amino acid sequence. In some embodiments, the DNA molecule does not comprise a cell penetrating polypeptide. For example, in some embodiments, the DNA molecule does not comprise a fusion protein that comprises a cell penetrating polypeptide.

In some embodiments, an effector described herein comprises an immunogen. In some embodiments, an effector described herein comprises a viral antigen, a bacterial antigen, a fungal antigen, or a tumor antigen. In some embodiments, an effector described herein comprises a peptide antigen. In some embodiments, a composition described herein is administered to a subject as a vaccine. In some aspects, the present disclosure provide a method of vaccinating a subject, comprising administering to a subject a composition described herein.

### RNA Effectors:

An effector sequence may be a DNA sequence encoding a non-coding RNA, e.g., one or more of a short interfering RNA (siRNA), a microRNA (miRNA), a long non-coding RNA, a piwi-interacting RNA (piRNA), a small nucleolar RNA (snoRNA), a small Cajal body-specific RNA (scaRNA), a transfer RNA (tRNA), a ribosomal RNA (rRNA), an RNA aptamer, and a small nuclear RNA (snRNA). In some embodiments, a DNA molecule described herein comprises a sequence encoding an RNA (e.g., an mRNA, siRNA, or miRNA). In some embodiments, a DNA molecule described herein does not comprise a sequence encoding an RNA.

In some embodiments, the DNA molecule disclosed herein comprises one or more expression sequences that encode a regulatory RNA, e.g., an RNA that modifies expression of an endogenous gene and/or an exogenous gene. In some embodiments, the DNA molecule disclosed herein can comprise a sequence that is antisense to a regulatory nucleic acid like a non-coding RNA, such as, but not limited to, tRNA, lncRNA, miRNA, IRNA, snRNA, microRNA, siRNA, piRNA, snoRNA, snRNA, exRNA, scaRNA, Y RNA, and hnRNA. In one embodiment, the regulatory nucleic acid targets a host gene. A regulatory nucleic acid may include, but is not limited to, a nucleic acid that hybridizes to an endogenous gene, e.g., an antisense RNA, a guide RNA, a nucleic acid that hybridizes to an exogenous nucleic acid such as a viral DNA or RNA, nucleic acid that hybridizes to an RNA, nucleic acid that interferes with gene transcription, nucleic acid that interferes with RNA translation, nucleic acid that stabilizes RNA or destabilizes RNA such as through targeting for degradation, or nucleic acid that modulates a DNA or RNA binding factor. In one embodiment, the sequence is a miRNA. In some embodiments, the regulatory nucleic acid targets a sense strand of a host gene. In some embodiments, the regulatory nucleic acid targets an antisense strand of a host gene.

In some embodiments, the DNA molecule disclosed herein encodes a guide RNA. Guide RNA sequences are generally designed to have a sequence having a length of between 15-30 nucleotides (e.g., 17, 19, 20, 21, 24 nucleotides) that is complementary to the targeted nucleic acid sequence, and a region that facilitates complex formation (e.g., with a tracrRNA or a nuclease). Custom gRNA generators and algorithms are available commercially for use in the design of effective guide RNAs. Gene editing has also been achieved using a chimeric “single guide RNA” (“sgRNA”), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing). Chemically modified sgRNAs have also been demonstrated to be effective in genome editing; see, for example, Hendel et al. (2015) Nature Biotechnol., 985-991. The gRNA may recognize specific DNA sequences (e.g., sequences adjacent to or within a promoter, enhancer, silencer, or repressor of a gene). In one embodiment, the gRNA is used as part of a CRISPR system for gene editing. For the purposes of gene editing, the DNA molecule disclosed herein may be designed to include one or multiple sequences encoding guide RNA sequences corresponding to a desired target DNA sequence; see, for example, Cong et al. (2013) Science, 339:819-823; Ran et al. (2013) Nature Protocols, 8:2281-2308.

A DNA molecule disclosed may encode certain regulatory nucleic acids that can inhibit gene expression through the biological process of RNA interference (RNAi). RNAi molecules comprise RNA or RNA-like structures typically containing 15-50 base pairs (such as about 18-25 base pairs) and having a nucleobase sequence identical (complementary) or nearly identical (substantially complementary) to a coding sequence in an expressed target gene within the cell. Such RNAi molecules include, but are not limited to: short interfering RNAs (siRNAs), double-strand RNAs (dsRNA), micro RNAs (miRNAs), short hairpin RNAs (shRNA), meroduplexes, dicer substrates (U.S. Pat. Nos. 8,084,599 8,349,809 and 8,513,207), and RNA antisense oligonucleotides (RNA ASOs).

In one embodiment, a DNA molecule disclosed herein comprises a sequence comprising a sense strand of a lncRNA. In one embodiment, a DNA molecule disclosed herein comprises a sequence encoding an antisense strand of a lncRNA.

A DNA molecule disclosed herein may encode a regulatory nucleic acid substantially complementary, or fully complementary, to a fragment of an endogenous gene or gene product (e.g., mRNA). The regulatory nucleic acids may complement sequences at the boundary between introns and exons, in between exons, or adjacent to exon, to prevent the maturation of newly-generated nuclear RNA transcripts of specific genes into mRNA for transcription. The regulatory nucleic acids that are complementary to specific genes can hybridize with the mRNA for that gene and prevent its translation. The antisense regulatory nucleic acid can be DNA, RNA, or a derivative or hybrid thereof. In some embodiments, the regulatory nucleic acid comprises a protein-binding site that can bind to a protein that participates in regulation of expression of an endogenous gene or an exogenous gene.

In some embodiments, a DNA molecule disclosed herein comprises an effector sequence encoding a regulatory nucleic acid that hybridizes to a transcript of interest, wherein the effector sequence has a length between about 5 to 30 nucleotides, between about 10 to 30 nucleotides, or about 11, 12, 13, 14, 15, 16, 17, 18, 19, 20, 21, 22, 23, 24, 25, 26, 27, 28, 29, 30 or more nucleotides. The degree of identity of the regulatory nucleic acid to the targeted transcript should be at least 75%, at least 80%, at least 85%, at least 90%, or at least 95%.

A DNA molecule disclosed herein may encode a micro-RNA (miRNA) molecule identical to about 5 to about 30 contiguous nucleotides of a target gene. In some embodiments, the miRNA sequence targets a mRNA and commences with the dinucleotide AA, comprises a GC-content of about 30-70% (about 30-60%, about 40-60%, or about 45%-55%), and does not have a high percentage identity to any nucleotide sequence other than the target in the genome of the mammal in which it is to be introduced, for example as determined by standard BLAST search. In some embodiments, the DNA molecule disclosed herein encodes at least one miRNA, e.g., 2, 3, 4, 5, 6, or more. In some embodiments, the DNA molecule disclosed herein comprises a sequence that encodes an miRNA having at least about 75%, at least about 80%, at least about 85%, at least about 90% at least about 95%, at least about 96%, at least about 97%, at least about 98%, at least about 99% or 100% nucleotide sequence identity to any one of the nucleotide sequences or a sequence that is complementary to a target sequence. Lists of known miRNA sequences can be found in databases maintained by research organizations, such as Wellcome Trust Sanger Institute, Penn Center for Bioinformatics, Memorial Sloan Kettering Cancer Center, and European Molecule Biology Laboratory, among others. Known effective siRNA sequences and cognate binding sites are also well represented in the relevant literature. RNAi molecules are readily designed by technologies known in the art. In addition, there are computational tools that increase the chance of finding effective and specific sequence motifs (see, e.g., Lagana et al., Methods Mol. Bio., 2015, 1269:393-412).

The DNA molecule disclosed herein may modulate expression of RNA encoded by a gene. Because multiple genes can share some degree of sequence homology with each other, in some embodiments, the DNA molecule disclosed herein can be designed to target a class of genes with sufficient sequence homology. In some embodiments, the DNA molecule disclosed herein can contain a sequence that has complementarity to sequences that are shared amongst different gene targets or are unique for a specific gene target. In some embodiments, the DNA molecule disclosed herein can be designed to target conserved regions of an RNA sequence having homology between several genes thereby targeting several genes in a gene family (e.g., different gene isoforms, splice variants, mutant genes, etc.). In some embodiments, the DNA molecule disclosed herein can be designed to target a sequence that is unique to a specific RNA sequence of a single gene.

In embodiments, the effector sequence encoding a regulatory RNA has a length less than 5000 bps (e.g., less than 4000 bps, less than 3000 bps, less than 2000 bps, less than 1000 bps, less than 900 bps, less than 800 bps, less than 700 bps, less than 600 bps, less than 500 bps, less than 400 bps, less than 300 bps, less than 200 bps, less than 100 bps, less than 50 bps, less than 40 bps, less than 30 bps, less than 20 bps, less than 10 bps, or less). In some embodiments, the effector sequence has, independently or in addition to, a length of at least 10 bps (e.g., at least 20 bps, at least 30 bps, at least 40 bps, at least 50 bps, at least 60 bps, at least 70 bps, at least 80 bps, at least 90 bps, at least 100 bps, at least 200 bps, at least 300 bps, at least 400 bps, at least 500 bps, at least 600 bps, at least 700 bps, at least 800 bps, at least 900 bps, at least 1000 kb, at least 1.1 kb, at least 1.2 kb, at least 1.3 kb, at least 1.4 kb, at least 1.5 kb, at least 1.6 kb, at least 1.7 kb, at least 1.8 kb, at least 1.9 kb, at least 2 kb, at least 2.1 kb, at least 2.2 kb, at least 2.3 kb, at least 2.4 kb, at least 2.5 kb, at least 2.6 kb, at least 2.7 kb, at least 2.8 kb, at least 2.9 kb, at least 3 kb, at least 3.1 kb, at least 3.2 kb, at least 3.3 kb, at least 3.4 kb, at least 3.5 kb, at least 3.6 kb, at least 3.7 kb, at least 3.8 kb, at least 3.9 kb, at least 4 kb, at least 4.1 kb, at least 4.2 kb, at least 4.3 kb, at least 4.4 kb, at least 4.5 kb, at least 4.6 kb, at least 4.7 kb, at least 4.8 kb, at least 4.9 kb, at least 5 kb or greater).

In some embodiments, a DNA molecule disclosed herein comprises one or more of the features described herein, e.g., one or more structural DNA sequence, a sequence encoding one or more peptides or proteins, a sequence encoding one or more regulatory element, a sequence encoding one or more regulatory nucleic acids, e.g., one or more non-coding RNAs, other expression sequences, and any combination of the aforementioned. A DNA molecule described herein may have one or a plurality of effector sequences, e.g., 2, 3, 4, 5 or more effector sequences. In the case of a plurality of effector sequences in a single DNA molecule, the effector sequences may be the same or different.

In some embodiments, the DNA molecule includes a therapeutically functional, structural DNA sequence. In some embodiments, the DNA molecule includes a promoter sequence and an effector sequence encoding a therapeutic peptide, polypeptide, or protein described herein. In some embodiments, the DNA molecule includes a promoter sequence and an effector sequence encoding a regulatory RNA described herein.

In some embodiments, the effector sequence that encodes a polypeptide or protein is codon optimized, e.g., codon optimized for expression in a mammal, e.g., a human. In general, codon optimization means modifying a nucleic acid sequence for enhanced expression in the host cells of interest by replacing at least one codon (e.g., one or more, e.g., 1, 2, 3, 4, 5, 10, 15, 20, 25, 50, or more codons; e.g., at least 1%, at least 5%, at least 10%, at least 20%, at least 25%, at least 50%, at least 60%, at least 70%, at least 80%, at least 90% or 100%) of the native sequence with codons that are more frequently or most frequently used in the genes of that host cell while maintaining the native amino acid sequence. Codon usage tables are available, for example, at the “Codon Usage Database” available at www.kazusa.or.jp/codon/. These tables can be adapted in a number of ways, see, e.g., Nakamura et al., 2000, Nucl. Acids Res. 28:292. Computer algorithms for codon optimizing a particular sequence for expression in a particular host cell are also available, such as Gene Forge.

### Maintenance Sequence

A DNA molecule disclosed herein may include a maintenance sequence that supports or enables sustained gene expression through successive rounds of cell division and/or progenitor differentiation in a host cell for a DNA described herein. In embodiments, a maintenance sequence is a nuclear scaffold/matrix attachment region (S/MAR). S/MAR elements are diverse, AT-rich sequences ranging from 60-500 bp that are conserved across species, thought to anchor chromatin to nuclear matrix proteins during interphase (Bode et al. 2003. Chromosome Res 11, 435-445). An S/MAR can be incorporated into a DNA molecule described herein to facilitate long-term transgene expression and extra-chromosomal maintenance. In one embodiment, the maintenance sequence is human interferon-beta MAR (5′tataattcactggaatttttttgtgtgtatggtatgacatatgggttcccttttattttttacatataaatatatttccctgtttttctaaaaaagaaaa agatcatcattttcccattgtaaaatgccatatttttttcataggtcacttacata-3′ (SEQ ID NO: 5)), or a functional sequence having at least 80%, at least 90%, at least 95%, or at least 98% identity thereto. In embodiments, S/MARs useful in the constructs described herein can be found by searching the MARome at bioinfo.net.in/MARome, described also by Narwade et al. 2019. Nucleic Acids Research. Volume 47, Issue 14:7247-7261.

In embodiments, a DNA molecule described herein is capable of replicating in a mammalian cell, e.g., human cell. In some embodiments, a DNA molecule described herein is maintained in a host cell, tissue or subject through at least one cell division. For example, a DNA molecule described herein is maintained in a host cell, tissue or subject through at least 2, at least 3, at least 4, at least 5, at least 6, at least 7, at least 8, at least 10, at least 15, at least 20, at least 40, at least 50 or more cell divisions. In vitro, cell division may be tracked by flow cytometry or microscopy. In vivo, cell division may be tracked by intravital microscopy.

### Other Elements

A DNA molecule disclosed herein may also include other control elements operably linked to the effector sequence, e.g., the sequence encoding an effector, in a manner which permits its transport, localization, transcription, translation and/or expression in a target cell, or which promotes its degradation or repression of expression in a non-target cell. As used herein, “operably linked” sequences include both expression control sequences that are contiguous with the sequence encoding the effector and expression control sequences that act in trans or at a distance to control the sequence encoding the effector. The precise nature of regulatory sequences needed for gene expression in host cells may vary between species, tissues or cell types, but in general may include, as necessary, 5′ non-transcribed and 5′ non-translated sequences involved with the initiation of transcription and translation respectively, such as a TATA box, capping sequence, CAAT sequence, enhancer elements and the like. Regulatory sequences may also include enhancer sequences or upstream activator sequences as desired. The constructs described herein may optionally include 5′ leader or signal sequences. In some embodiments, a DNA molecule described herein may comprise a sequence encoding a 5′ untranslated region (5′ UTR) and/or a sequence encoding a 3′ untranslated region (3′ UTR). In some embodiments, the DNA molecule encodes an intron sequence. In some embodiments, the DNA molecule comprises a sequence encoding a polyadenylation site. In some embodiments, the DNA molecule comprises an enhancer sequence.

The DNA molecule may comprise a non-coding region. In some embodiments, the non-coding region is completely free of predicted ORFs. In some embodiments, the non-coding region does not encode a protein sequence. In some embodiments, the non-coding region is not translated or is not translated at a substantial level.

### Structural Elements of RNA Molecules

In some embodiments, an RNA molecule (e.g., mRNA molecule) described herein is linear and single stranded. In some embodiments, the RNA molecule comprises two free ends.

In some embodiments, the RNA molecule is a circular single-stranded RNA molecule, in which the RNA molecule lacks a free end. A circular RNA molecule may be covalently closed or may form a closed structure without free RNA ends through non-covalent interactions, e.g., the RNA molecule may be closed through a splint, e.g., a nucleic acid (e.g., DNA or RNA) splint, through a moiety such as a protein that binds and brings together both ends of a linear RNA molecule, or through binding of a plurality of proteins, each of two of the plurality binding to a different RNA end, and then binding to each other or a third moiety to close the RNA structure. In the case of circular RNA molecules, the term circular does not imply that the RNA structure lacks all intramolecular structure; rather, a circular RNA molecule may have short regions of intramolecular double stranded regions or other structures.

An RNA molecule being single stranded does not imply that it is entirely devoid of any intramolecular base pairing. In some embodiments, a single-stranded RNA molecule described herein may have less than a threshold level of intramolecular complementarity or double stranded structures. In some embodiments, the single stranded RNA molecule does not comprise more than 10, more than 8, more than 7, more than 5, more than 4, more than 3, more than 2, or more than 1 double stranded region longer than 20, 15, 10, or 5 base pairs. In some embodiments, the single stranded RNA molecule does not comprise more than 10, more than 8, more than 7, more than 5, more than 4, more than 3, more than 2, or more than 1 double stranded region longer than 20 base pairs. In some embodiments, the single stranded RNA molecule does not comprise any regions of intramolecular complementarity longer than 20, 15, 10, or 5 base pairs. In some embodiments, the single stranded RNA molecule does not comprise any regions of intramolecular complementarity longer than 20 base pairs. In some embodiments, the single stranded RNA molecule comprises 1, 2, 3, 4, 5, 7, 8, or 10, double stranded regions, e.g., wherein the double stranded regions are no more than 20, 15, 10, or 5 base pairs.

In some embodiments, the single-stranded RNA molecule does not form a double stranded structure longer than 20 base pairs. In some embodiments, the single-stranded RNA molecule does not comprise a first sequence that hybridizes with a second sequence, wherein the first sequence and the second sequence are at least 5, at least 10, at least 15, at least 20, or at least 25 nt long, and wherein the first sequence and the second sequence are positioned less than 6, 5, 4, 3, 2, or 1 nucleotides apart from each other.

In some embodiments, the RNA molecule is double stranded. The RNA molecule may be double stranded and circular. The RNA molecule may be double stranded and linear.

In some embodiments, the RNA molecule has a GC content of 30-40%, 40-50%, 50-60%, or 60-70%. In some embodiments, the RNA molecule lacks a viral packaging site, and/or the RNA molecule does not encode a viral capsid gene.

In some embodiments, an RNA molecule disclosed herein is at least 50 nucleotides, at least 75 nucleotides, at least 100 nucleotides, at least 200 nucleotides, at least 300 nucleotides, at least 400 nucleotides, at least 500 nucleotides, or at least 1000 nucleotides in length. In some embodiments, the RNA molecule disclosed herein is between 40-50, 50-75, 75-100, 100-200, 200-300, 300-400, 400-500, or 500-1000 nucleotides in length. In some embodiments, the size of an RNA molecule disclosed herein is a length sufficient to encode one or more useful polypeptides.

In some embodiments, at least 80%, at least 85%, at least 90%, at least 95%, at least 98%, at least 99%, or 100% of the sugars of an RNA molecule described herein comprise-OH at their 2′ position.

In some embodiments, an RNA molecule described herein is recognized by a ribosome. In some embodiments, an RNA molecule described herein is translated, e.g., in a cell or a lysate.

In some embodiments, the single stranded RNA molecule is a sense strand. In some embodiments, the single stranded RNA molecule is an antisense strand.

mRNAs

In some embodiments, an RNA molecule described herein is a messenger RNA (mRNA). Typically, an mRNA molecule has sufficient elements to be recognized by a ribosome and direct translation of a polypeptide. In some embodiments, the mRNA molecule comprises a cap. In some embodiments, the mRNA molecule comprises a polyA tail situated 3′ of the effector sequence.

A 5′ cap may be canonical or chemically modified. In some embodiments, the mRNA molecule comprises one or more of: anti-reverse cap analog (ARCA; m27.3′-OGP3G), GP3G (Unmethylated Cap Analog), m7GP3G (Monomethylated Cap Analog), m32.2.7GP3G (Trimethylated Cap Analog), m5CTP (5′-methyl-cytidine triphosphate), m6ATP (N6-methyl-adenosine-5′-triphosphate), s2UTP (2-thio-uridine triphosphate), Ψ (pseudouridine triphosphate), a hypermethylated cap analog; an NAD+-derived cap analog (e.g., as described in Kiledjian, Trends in Cell Biology 28, 454-464 (2018)); or a modified, e.g., biotinylated, cap analog (e.g., as described in Bednarek et al., Phil Trans R Soc B 373, 20180167 (2018)).

In some embodiments, the mRNA molecule comprises a 7-methylguanosine cap (e.g., a O-Me-m7G cap).

In some embodiments, the mRNA molecule comprises a 3′ polyA tail. In some embodiments, the 3′ polyA tail has a length of 50-100, 100-200, or 200-300 ribonucleotides. In some embodiments, the nucleobases in the 3′ poly A tail are exclusively adenine.

In some embodiments, an mRNA molecule described herein has a sequence that is identical to a DNA molecule described herein. It is understood that the two sequences will still be considered identical even if the mRNA molecule comprises U at every position where the DNA molecule comprises T.

### Sequence Elements of RNA Molecules

In some embodiments, an RNA molecule described herein comprises an effector sequence. The effector sequence may either encode an effector (e.g., a polypeptide) or may itself be a functional RNA sequence. In some embodiments, an RNA molecule described herein comprises a heterologous functional sequence. In some embodiments, the RNA molecule comprises both of an effector sequence and a heterologous functional sequence.

In some embodiments, the effector sequence encodes a polypeptide (e.g., a protein). In some embodiments, the effector sequence is heterologous to a target cell.

The RNA molecule described herein may also include other native or heterologous expression control elements, such as a polyA tail or Kozak consensus sequences.

### Effector Sequence

### Polypeptide Effectors:

The effector sequence of an RNA molecule described herein (e.g., an mRNA molecule described herein) may be an RNA sequence encoding a therapeutic peptide, polypeptide or protein. In some embodiments, a polypeptide effector encoded by RNA molecule described herein is an immunogen.

In embodiments, the RNA molecule can include a plurality of effector sequences. In some embodiments, the RNA molecule comprises a second effector sequence which is the same as or different than the first effector sequence. An RNA molecule can include an effector sequence that is a functional RNA and a second effector sequence that is an RNA sequence encoding a functional polypeptide. The plurality of effector sequences may be the same or different sequences of the same type. For example, a sequence described herein may include multiple sequences encoding multiple proteins, e.g., a plurality of proteins in a biological pathway.

In some embodiments, an RNA molecule or sequence described herein may include a plurality of sequences encoding a polypeptide, separated by a self-cleaving peptide, e.g., P2A, T2A, E2A or F2A. In some embodiments, an RNA molecule or sequence described herein may include a sequence encoding a first polypeptide of interest, followed by a sequence encoding a 2A self-cleaving peptide, a sequence encoding a second polypeptide of interest, and a polyA tail.

In some embodiments, the effector sequence that encodes a polypeptide or protein is codon optimized, e.g., codon optimized for expression in a mammal, e.g., a human.

In some embodiments, an RNA molecule described herein may comprise an effector sequence encoding one or more effectors. In some embodiments, the effector comprises a peptide or protein selected from the sub-section above entitled “Polypeptide effectors” within the section “Sequence elements of DNA molecules.”

An RNA molecule described herein may comprise an effector sequence selected from the sub-section above entitled “RNA effectors” within the section “Sequence elements of DNA molecules.”

### Click Handles, Click Linkers, and Click Reactions

In some embodiments, click chemistry is used to link a first moiety to a second moiety as described herein. For example, in some embodiments, a composition described herein comprises a LNP and a targeting moiety, wherein the targeting moiety is connected to the LNP via a click linker. In some embodiments, an LNP described herein is linked to a targeting moiety through one or more covalent bonds. In some embodiments, an LNP described herein is linked to a targeting moiety through a linker, e.g., via a click linker. In some embodiments, the click linker is formed as a product of a click reaction between a first click handle and a second click handle. A variety of reactions that fulfill the criteria for click chemistry are known, a wide range of commercially available reagents for click chemistry can be used, and one skilled in the art could use (for example) any one of a number of published methodologies (see, e.g., pubs.acs.org/doi/10.1021/acs.chemrev.1c00469 or www.ncbi.nlm.nih.gov/pmc/articles/PMC2562613/, which are herein incorporated by reference in their entirety). In some embodiments, conjugation is performed using click chemistry.

### Click Handles

For example, in some embodiments, a first click handle is bound to an LNP. For example, the first click handle may be covalently bound (e.g., directly bound) to an LNP. In some embodiments, a second click handle is bound to a targeting moiety. For example, in the case of a targeting moiety comprising a polypeptide, the second click handle may be covalently bound (e.g., directly bound) to an amino acid of the targeting moiety, such as a lysine of the targeting moiety. The LNP and first click handle may be contacted with the targeting moiety and second click handle under conditions that allow the first click handle to react with the second click handle in a click reaction.

A variety of molecules that comprise a click handle can be used. For instance, in some embodiments, the molecule is chosen from:

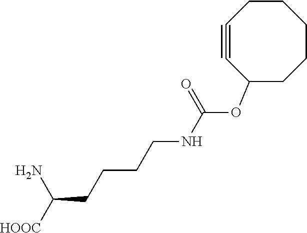 [SMILES: N[C@@H](CCCCNC(=O)OC1C#CCCCCC1)C(=O)O]

SCO-Lysine, e.g., for use in a strain-promoted alkyne-azide cycloaddition (SPAAC) reaction or a strain-promoted inverse-electron-demand Diels-Alder cycloaddition (SPIEDAC) reaction;

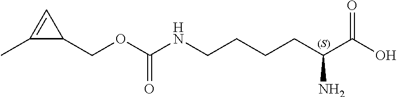

Cyclopropene lysine, e.g., for use in a SPIEDAC reaction;

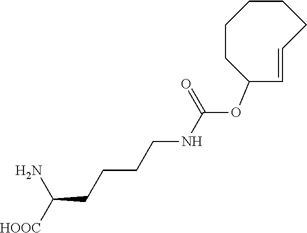 [SMILES: N[C@@H](CCCCNC(=O)OC1/C=C/CCCCC1)C(=O)O]

TCO*A-Lysine, e.g., for use in a SPIEDAC reaction;

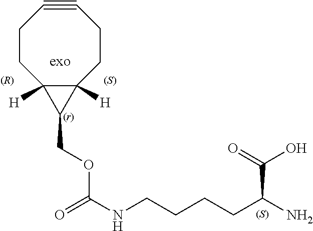

Exo-BCN-Lysine, e.g., for use in a SPAAC or SPIEDAC reaction;

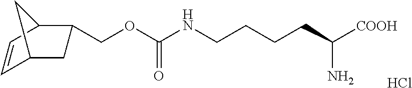 [SMILES: N[C@@H](CCCCNC(=O)OCC1CC2C=CC1C2)C(=O)O ; Cl]

NBO-Lysine, e.g., for use in a SPIEDAC reaction;

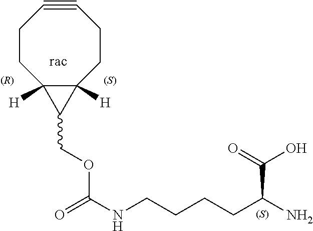

rac-BCN-Lysine, e.g., for use in a SPAAC or SPIEDAC reaction;

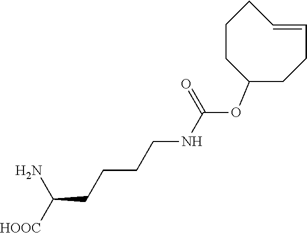 [SMILES: N[C@@H](CCCCNC(=O)OC1CC/C=C/CCC1)C(=O)O]

TCO-Lysine, e.g., for use in a SPIEDAC reaction;

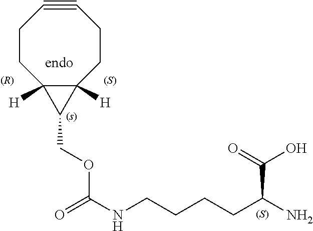

Endo-BCN-Lysine, e.g., for use in a SPAAC or SPIEDAC reaction

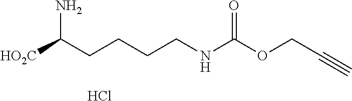 [SMILES: C#CCOC(=O)NCCCC[C@H](N)C(=O)O ; Cl]

PrK-HCl-salt, e.g., for use in a copper-catalyzed azide-alkyne cycloaddition (CuAAC) reaction;

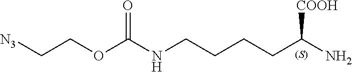

N3-Lysine, e.g., for use in a SPAAC or SPIEDAC reaction;

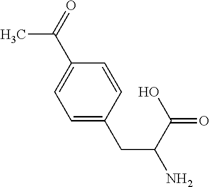

p-acetylphenylalanine, e.g., for use with a site-specific oxime ligation (e.g., mediated by DBCO-amine), followed by SPAAC;

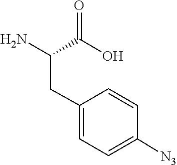 [SMILES: [N-]=[N+]=Nc1ccc(C[C@H](N)C(=O)O)cc1]

p-azidomethylphenylalanine, e.g., for use in a SPAAC or SPIEDAC reaction;

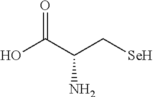

seleno-cysteine, e.g., for reaction with maleimide; or

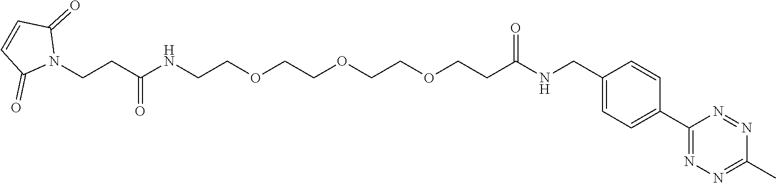 [SMILES: Cc1nnc(-c2ccc(CNC(=O)CCOCCOCCOCCNC(=O)CCN3C(=O)C=CC3=O)cc2)nn1]

methyltetrazine-PEG3-maleimide, e.g., for use in SPIEDAC or reaction with thiol;

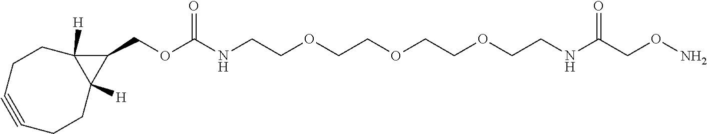 [SMILES: NOCC(=O)NCCOCCOCCOCCNC(=O)OC[C@H]1[C@@H]2CCC#CCC[C@@H]21]

BCN-PEG3-oxyamine, e.g., for use in SPAAC or SPIEDAC reaction or an oxime ligation.

A click handle may comprise an azide or an alkyne. In some embodiments, an LNP described herein is linked to a first click handle that reacts specifically with a second click handle linked to a targeting moiety, thereby producing a click linker between the LNP and the targeting moiety. In some embodiments, the first click handle comprises an azide moiety, and the second click handle comprises an alkyne moiety. In some embodiments, the first click handle comprises an alkyne moiety, and the second click handle comprises an azide moiety.

In some embodiments, the click handle comprises an alkyne moiety. In some embodiments, the alkyne moiety comprises a propargyl moiety or a cyclooctynyl moiety. Exemplary alkyne moieties include diarylcyclooctyne (DBCO)-sulfo-NHS-ester, diarylcyclooctyne (DBCO)-PEG-NHS-ester, diarylcyclooctyne (DBCO)-C6-NHS-ester, diarylcyclooctyne (DBCO)-NHS-ester, diarylcyclooctyne (DBCO)-amine, diarylcyclooctyne (DBCO)-acid, sulfo diarylcyclooctyne (DBCO)-maleimide, diarylcyclooctyne (DBCO)-maleimide, bis-sulfone-PEG-diarylcyclooctyne (DBCO), propargyl-NHS ester, propargyl-maleimide, alkyne-PEG-NHS ester, alkyne-PEG-maleimide, or a derivative thereof. In some embodiments, the alkyne moiety comprises DBCO provided as a dibenzocyclooctyne-acid (CAS 1353016-70-2).

In some embodiments, the click handle comprises an azide moiety. In some embodiments, the azide moiety comprises an azidoalkyl moiety, azidoaryl moiety, or an azidoheteroaryl moiety. Exemplary azide moieties include 3-azidopropionic acid sulfo-NHS ester, azidoacetic acid NHS ester, azido-PEG-NHS ester, azidopropylamine, azido-PEG-amine, azido-PEG-maleimide, bis-sulfone-PEG-azide, or a derivative thereof.

Click handles may also comprise an alkene moiety, e.g., a transcycloalkene moiety, an oxanorbornadiene moiety, or a tetrazine moiety. Additional click handles can be found in Click Chemistry Tools (clickchemistrytools.com), Lahann, J (ed) (2009) Click Chemistry for Biotechnology and Materials Science, Mckay et al, “Click chemistry in complex mixtures: bioorthogonal bioconjugation” Chem Biol. 2014 Sep. 18; 21(9):1075-101, Becer et al. “Click chemistry beyond metal-catalyzed cycloaddition” Angew Chem Int Ed Engl. 2009; 48(27):4900-8., and Hein et al. “Click chemistry, a powerful tool for pharmaceutical sciences” Pharm Res. 2008 October; 25(10):2216-30, each of which is incorporated herein by reference in its entirety.

In embodiments, the click handle comprises a tetrazine moiety, e.g., for reaction with an alkene moiety. For instance, in embodiments, the tetrazine is a 1,2,4,5 tetrazine and the alkene is a strained alkene. In embodiments, the alkene moiety comprises a trans-cyclooctene, (E)-Cyclooct-4-enol, (E)-Cyclooct-4-enyl 2,5-dioxo-1-pyrrolidinyl carbonate, 5-Norbornene-2-acetic acid succinimidyl ester, 5-Norbornene-2-endo-acetic acid, TCO PEG4 succinimidyl ester, TCO-amine, or TCO-PEG3-maleimide. In embodiments, the tetrazine click handle comprises (4-(1,2,4,5-Tetrazin-3-yl)phenyl) methanamine or 2,5-Dioxo-1-pyrrolidinyl 5-[4-(1,2,4,5-tetrazin-3-yl)benzylamino]-5-oxopentanoate, 5-[4-(1,2,4,5-Tetrazin-3-yl)benzylamino]-5-oxopentanoic acid. In embodiments, the tetrazine and alkene react in a Diels-Alder cycloaddition to yield a stable covalent linkage. In embodiments, a catalyst is not needed. In embodiments, the only byproduct is dinitrogen. In embodiments, the reaction at least one order of magnitude faster than azide-cyclooctyne based click chemistry. Without wishing to be bound by theory, tetrazine/alkene reactions can be used with low concentrations of reactant.

In some embodiments, the click handles react via an azide-alkyne Huisgen cycloaddition. In some embodiments, an azide-alkyne Huisgen cycloaddition comprises a copper (I)-catalyzed azide-alkyne cycloaddition or a strain-promoted azide-alkyne cycloaddition.

In some embodiments, the click handles react to form a heteroaryl, e.g., a triazole. In some embodiments, the triazole comprises a 1,2,3-triazole, e.g., a 1,4-disubstituted 1,2,3-triazole or a 1,5-disubstituted 1,2,3-triazole.

In some embodiments, the click handle comprises an alkyne and reacts with an amine. In some embodiments, the click handle comprises a cyclooctyne and reacts with an amine. In some embodiments, the click handle comprises diarylcyclooctyne (DBCO)-sulfo-NHS-ester or diarylcyclooctyne (DBCO)-PEG5-NHS-ester.

In some embodiments, the click handle comprises an azide and reacts with an amine. In some embodiments, the click handle is 3-azidopropionic acid sulfo-NHS ester or azido-PEG4-NHS-ester.

In an embodiment, the click handle is water soluble. In an embodiment, the click handle is membrane impermeable, e.g., has sufficient charge to render it membrane impermeable. In an embodiment the click handle is charged, e.g., positively charged or negatively charged. In an embodiment, the click handle comprises a cationic moiety or an anionic moiety, e.g., a SO₃ moiety.

In some embodiments, the click handle comprises a detection agent, e.g., useful for detection of the DNA molecule. Exemplary detection agents may include a fluorescent molecule (e.g., a cyanine dye, e.g., Cy3, Cy 3.5, Cy5, Cy5.5, Cy7, or Cy7.5), a metal chelate, a contrast agent, a radionuclide, a positron emission tomography (PET) imaging agent, an infrared imaging agent, a near-IR imaging agent, a computer assisted tomography (CAT) imaging agent, a photon emission computerized tomography imaging agent (e.g., DIBO-DFO, where DFO chelates Zirconium-89), an X-ray imaging agent, or a magnetic resonance imaging (MRI) agent.

Suitable click handles may comprise, for example, an amine, sulfate, thiol, hydroxyl, azide, alkyne, alkene, carboxyl groups aldehyde groups, sulfone groups, vinylsulfone groups, isocyanate groups, acid anhydride groups, epoxide groups, aziridine groups, episulfide groups, groups such as —CO₂N(COCH₂)2, —CO₂N(COCH₂)₂, —CO₂H, —CHO, —CHOCH₂, —N═C—O, —SO₂CH≡CH₂, —N(COCH)₂, —S—S—(C₅H₄N) and groups of the following structures wherein X is halogen and R is hydrogen or C₁ to C₄ alkyl:

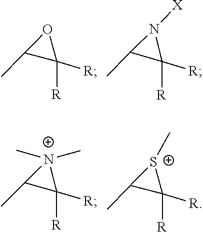

In an embodiment, a coupling reagent or a click handle is a GMP grade material.

### Click Linkers

Linkage of the two substrates (e.g., an LNP and a targeting moiety) typically results in a residual linker between the first and second substrate. For example, in the case of click handles comprising an azide and an alkyne, a residual linker may be formed comprising a triazole (e.g., a 1,2,3-triazole).

Exemplary click linkers suitable for use in the composition and methods described herein include, for example

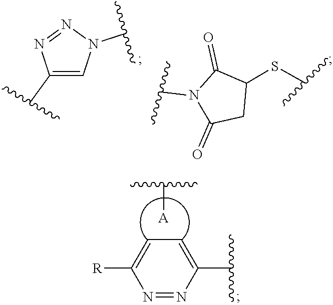

wherein R is H, a hydrocarbon or heteroatom which may be further optionally substituted, and A is a C₃-C₁₂ cycloalkane, cycloalkene, cycloalkyne, or a heterocycle, all of which may be further optionally substituted;

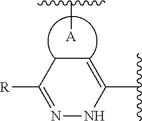

wherein R is H, a hydrocarbon or heteroatom which may be further optionally substituted, and A is a C₃-C₁₂ cycloalkane, cycloalkene, cycloalkyne, or a heterocycle, all of which may be further optionally substituted,

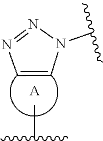

wherein A is a C₃-C₂₀ cycloalkene, cycloalkyne, or a heterocycle, all of which may be further optionally substituted,

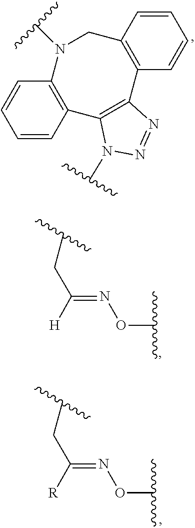

wherein R is a hydrocarbon which is further optionally substituted.

In an embodiment, the click linker is an alkyne/azide click linker (e.g., wherein the alkyne is a cyclooctyne, activated alkyne, or electron-deficient alkyne), e.g., the click linker comprises a triazole, e.g., a 1,2,3-triazole and/or a disubstituted triazole. In an embodiment, the click linker is a diene/dienophile click linker (e.g., wherein the dienophile comprises an alkene moiety), e.g., the click linker comprises a cycloalkene, e.g., a disubstituted alkene. In embodiments, the click linker is a tetrazine/alkene click linker, e.g., the click linker comprises a dihydropyrazine, e.g., a 1,2-dihydropyrazine. In embodiments, the click linker is a tetrazole/alkene click linker, e.g., the click linker comprises a diazole. In embodiments, the click linker is a dithioester/diene click linker, e.g., the click linker comprises a sulfur-containing ring, e.g., a tetrahdrothiophene, e.g., a disubstituted tetrahdrothiophene. In embodiments, the click linker is a dithioester/diene linker, e.g., the click linker comprises a sulfur-containing ring, e.g., a thiopyran. In embodiments, the click linker is a thiol/alkene click linker, e.g., the click linker comprises an alkyl sulfide.

### Click Reactions

In some embodiments, a method described herein comprises a step of performing a click reaction. In some embodiments, a tLNP composition described herein is produced using a click reaction.

In some embodiments, the click reaction is a cycloaddition (e.g., a 1,3-dipolar cycloaddition or hetero-Diels-Alder cycloaddition), nucleophilic ring-opening (e.g., openings of strained heterocyclic electrophiles such as aziridines, epoxides, cyclic sulfates, aziridinium ions, and episulfonium ions), carbonyl chemistry of no-aldol type (e.g., formation of ureas, thioureas, hydrazones, oxime ethers, amides, or aromatic heterocycles), or an addition to a carbon-carbon multiple bond (e.g., epoxidation, aziridination, dihydroxylation, sulfenyl halide addition, nitosyl halide addition, or Michael addition). Examples of these types of click reaction are described in greater detail in Hein et al., Pharm. Res. 2008 October; 25(10):2216-2230, which is herein incorporated by reference in its entirety. In embodiments, the click reaction is a metal-free [3+2] cycloaddition reaction, Diels-Alder reaction, or thiol-alkene radical reaction. Examples of these types of click reaction are described in greater detail in Becer et al., Angew. Chem. Int. Ed. 2009, 48, 4900-4908, which is herein incorporated by reference in its entirety.

In embodiments, the click reaction does not require a catalyst. In embodiments, the click reaction does not require copper ions, e.g., proceeds at substantially the same rate in the absence of copper ions as in the presence of copper ions, e.g., under conditions described in Tornoe, C. W. et al (2002). “Peptidotriazoles on Solid Phase: [1,2,3]-Triazoles by Regiospecific Copper (I)-Catalyzed 1,3-Dipolar Cycloadditions of Terminal Alkynes to Azides”. In embodiments, the click reaction proceeds efficiently at a temperature of about 10-40, 20-40, 20-30, 20-25, 30-40, or 35-40, or about 37 C. In embodiments, the click reaction proceeds efficiently at a temperature of below 50, 45, 40, 35, 30, 25, or 20C.

In embodiments, the activation barrier for a click reaction is 24-30, 25-29, or 26-28 kcal/mol, e.g., about 27.8 kcal/mol or 26 kcal/mol. In embodiments, the activation barrier for a click reaction is the same as or no less than 50%, 40%, 30%, 20%, or 10%, different from the activation barrier of a Huisgen Cu-catalyzed cycloaddition reaction between an azide and a terminal alkene, e.g., as described in Hein et al. Click chemistry, a powerful tool for pharmaceutical sciences” Pharm Res. 2008 October; 25(10):2216-30.

In embodiments, the click reaction is exergonic, e.g., having a ΔG° of between −10 and −100, −20 and −90, −30 and −70, −40 and −70, −50 and −60, or about −61 kcal/mol. In embodiments, the ΔG° for a click reaction is the same as or no less than 50%, 40%, 30%, 20%, or 10%, different from the ΔG° of a Huisgen Cu-catalyzed cycloaddition reaction between an azide and a terminal alkene.

In embodiments, the click reaction has a ΔG° of between −30 and −140, −40 and −130, −50 and −120, −60 and −110, −70 and −100, −80 and −90, or about 84 kJ/mol.

One example of a cycloaddition reaction is the Huisgen 1,3-dipolar cycloaddition of a dipolarophile with a 1,3 dipolar component that produce five membered (hetero) cycles. Examples of dipolarophiles are alkenes, alkynes, and molecules that possess related heteroatom functional groups, such as carbonyls and nitriles. Specifically, another example is the 2+3 cycloaddition of alkyl azides and acetylenes. Other cycloaddition reactions include Diels-Alder reactions of a conjugated diene and a dienophile (such as an alkyne or alkene). Examples of cycloaddition reactions are described, e.g., in U.S. Pat. No. 9,517,291, which is herein incorporated by reference in its entirety.

Other examples of click reactions include a hydrosilation reaction of H—Si and simple non-activated vinyl compounds, urethane formation from alcohols and isocyanates, Menshutkin reactions of tertiary amines with alkyl iodides or alkyl trifluoromethanesulfonates, Michael additions, e.g., the very efficient maleimide-thiol reaction, atom transfer radical addition reactions between —SO₂Cl and an olefin (R¹, R²—C═C—R³, R⁴), metathesis, Staudinger reaction of phosphines with alkyl azides, oxidative coupling of thiols, nucleophilic substitution, especially of small strained rings like epoxy and aziridine compounds, carbonyl chemistry like formation of ureas, and addition reactions to carbon-carbon double bonds like dihydroxylation. Therefore, attached functionality may be chosen from acetylene bond, an azido-group, a nitrile group, acetylenic, amino group, phosphino group.

In some embodiments, a click reaction forms very energy-efficient carbon-heteroatom bonds, in particular a ring opening nucleophilic reaction or a cycloaddition reaction. A type of reaction which is widely represented in click chemistry is the abovementioned alkyne-azide cycloaddition catalyzed with Cu(I). Examples of click reactions are also described, e.g., in U.S. Pat. No. 9,453,843, which is herein incorporated by reference in its entirety.

Click chemistry may generate substances quickly and reliably by joining small modular units together (see, e.g., Kolb et al. (2001) Angewandte Chemie Intl. Ed. 40:2004-2011; Evans (2007) Australian J. Chem. 60:384-395; Carlmark et al. (2009) Chem. Soc. Rev. 38:352-362; each herein incorporated by reference in its entirety).

### Non-Covalent Bonds, e.g., Biotin-Avidin Interactions

In some embodiments, a lipid described herein, e.g., a lipid of an LNP described herein, is linked to a targeting moiety through a non-covalent bond, e.g., via an interaction between a biotin moiety (e.g., biotin) and an avidin moiety. In some embodiments, the lipid is linked to a biotin moiety, and the targeting moiety is linked to an avidin moiety. In some embodiments, the lipid is linked to an avidin moiety, and the targeting moiety is linked to a biotin moiety. In some embodiments, the lipid or targeting moiety is linked to a plurality of biotin moieties, e.g., two biotin moieties.

In some embodiments, the avidin moiety comprises a streptavidin, e.g., a wild-type strepatividin. In some embodiments, the streptavidin comprises an amino acid sequence of SEQ ID NO: 1, or a sequence having at least 80%, at least 85%, at least 90%, at least 95%, or at least 99% identity thereto. In some embodiments, the avidin moiety comprises a monovalent strepatividin.

(SEQ ID NO: 1)
MRKIVVAAIAVSLTTVSITASASADPSKDSKAQVSAAEAGITGTWYNQL
GSTFIVTAGADGALTGTYESAVGNAESRYVLTGRYDSAPATDGSGTALG
WTVAWKNNYRNAHSATTWSGQYVGGAEARINTQWLLTSGTTEANAWKST
LVGHDTFTKVKPSAASIDAAKKAGVNNGNPLDAVQQ.

### Chemically Modified Nucleotides

The DNA molecules described herein may have chemical modifications of the nucleobases, sugars, and/or the phosphate backbone. While not wishing to be bound by theory, such modifications can be useful for protecting a DNA from degradation (e.g., from exonucleases) or from the immune system of a host tissue or subject. In general, a chemically modified nucleotide has the same base-pairing specificity as the unmodified nucleotide, e.g., a chemically modified adenine “A” can base-pair with thymine “T”. One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, chemical modifications (e.g., one or more modifications) are present in each of the sugar and the internucleoside linkage.

Examples of chemical modifications to DNA useful in the methods described herein include, e.g., N6-Methyladenosine (m6A, 6 mA); 5-formylcytosine (5-formyl-2′-deoxycytosine, 5fC, f5C); 5-carboxylcytosine (5-carboxyl-2′-deoxycytosine, 5-carboxycytosine, ca5C, 5caC); 5-hydroxymethylcytosine (5-hydroxymethyl-2′-deoxycytosine, 5hmC, hm5C); 5-methyldeoxycytosine (5-methylcytosine; 5-methyl-2′-deoxycytosine; m5dC; 5mC, m5C); 5′-methylcytosine; 3-methylcytosine (m3C); 2′-fluoro-2′deoxynucleoside; 5-glucosylmethylcytosine; 5-methyl pyrimidine; 8-oxoguanine (8-oxoG); phosphorothioate; S and R phsophorothioate linkages; methylthymine; N3′-P5′ Phosphoroamidate (NP); cyclohexane nucleic acid (CeNA); tricyclo-DNA (tcDNA). See, e.g., Pu et al. 2020. An in-vitro DNA phosphorothioate modification reaction. Mol Microbiol. 113:452-463; Zheng & Sheng. 2021. Synthesis of N4-methylcytidine (m4C) and N4,N4-dimethylcytidine (m42C) modified RNA. Current Protocols, 1, e248; Ohkubo et al. 2021. Chemical synthesis of modified oligonucleotides containing 5′-amino-5′-deoxy-5′-hydroxymethylthymidine residues. Current Protocols, 1, e70; Bao & Xu. 2021. Observation of Z-DNA structure via the synthesis of oligonucleotide DNA containing 8-trifluoromethyl-2-deoxyguanosine. Current Protocols, 1, e28; Skakuj et al. 2020. Automated synthesis and purification of guanidine-backbone oligonucleotides. Current Protocols in Nucleic Acid Chemistry, 81, e110.

In some embodiments, a DNA molecule described herein comprises a nucleotide comprising a chemically modified cytosine nucleobase. In some embodiments, a DNA molecule described herein comprises a nucleotide comprising a uracil nucleobase. In some embodiments, a DNA molecule described herein comprises a nucleotide comprising a canonical uracil nucleobase. In some embodiments, a DNA molecule described herein comprises a nucleotide comprising a chemically modified uracil nucleobase. In some embodiments, a DNA molecule described herein comprises a nucleotide comprising 5-hydroxymethyluracil.

In some embodiments, a DNA molecule described herein comprises a sense strand and an antisense strand, wherein the antisense strand comprises one or more chemically modified nucleotides. In some embodiments, a DNA molecule described herein comprises a sense strand and an antisense strand, wherein the sense strand does not comprise any chemically modified nucleotides. In some embodiments, a DNA molecule described herein comprises a sense strand and an antisense strand, wherein the sense strand comprises one or more chemically modified nucleotides.

In some embodiments, a DNA molecule as described herein may comprise a phosphorothioate-modified nucleotide. In some embodiments, the DNA molecule described herein may include S and R phosphorothioate modified nucleotide linkages. In one embodiment, the phosphorothioate linkages are made according to Iwamoto et al, 2017, Nature Biotechnology, Volume 35:845-851. Briefly, monomers of nucleoside 3′-oxazaphospholidine derivates undergo stereocontrolled oligonucleotide synthesis with iterative capping and sulfurization to create stereocontrolled phosphorothioate linkages. The final sample is analyzed by reverse-phase high-performance liquid chromatography (RP-HPLC) and Ultraperformance liquid chromatography mass spectrometry (UPLC/MS) to determine stereochemistry of the modification. Nucleic acids containing phosphorothioate linkages are also commercially available.

In some embodiments, a DNA molecule described herein may include boranophosphate modified nucleotides, e.g., following the methods in Sergueev and Shaw, 1998, J Am Chem Soc, Volume 120, Issue 37:9417-9427. Briefly, H-phosphonate chain elongation is followed by boronation to substitute a borano group for a nonbridging oxygen in the phosphate backbone. The final sample is purified and analyzed by RP-HPLC to determine stereochemistry of the modification. Boranophosphate modified nucleotides are also commercially available.

In some embodiments, a DNA molecule described herein may include 5-methylcytosine modified nucleotides, e.g., made following the methods in Lin et al, 2002, Mol Cell Biol, Volume 22, Issue 3:704-723. Briefly, cytosine or the sequence containing cytosine is incubated with glutathione S-transferase fusion of wild-type Dnmt3a (GST-3a) protein using unlabeled S-adenosylmethionine (AdoMet). The nucleotides are purified and analyzed by HPLC to determine that the nucleotides are methylated at the correct position. 5-methylcytosine modified nucleotides are also available commercially.

In some embodiments, a DNA molecule described herein may include 7-methylguanine modified nucleotides. In one embodiment, 7-methylguanine modified nucleotides are made following the methods in Jones and Robins, 1963, Purine nucleosides. III. Methylation studies of certain naturally occurring purine nucleosides, J Am Chem Soc, Volume 85:193. Briefly, 2′-deoxyguanosine in dimethyl sulfoxide is treated with methyl iodide. The nucleotides are purified and analyzed by HPLC to determine that the nucleotides are methylated at the correct position. In another embodiment, 7-methylguanine modified nucleotides are made according to the methods described in Hendler et al, 1970, Volume 9, Issue 21:4141:4153, and Kore and Parmar, 2006, Biochemistry, Volume 25, Issue 3:337-340. Briefly, instead of guanosine 5′-diphosphate, guanine 5′-diphosphate in water is added to dimethyl sulfate to yield 7-methyl GDP. The nucleotides are purified and analyzed by HPLC to determine that the nucleotides are methylated at the correct position. 7-methylguanine modified nucleotides are also available commercially.

In some embodiments, a DNA molecule described herein comprises methylation at one or more CpG or GpC dinucleotide.

In some embodiments, a DNA molecule described herein comprises a carboxyl modification or a formyl modification.

In embodiments, a DNA molecule described herein, or one strand (e.g., the sense strand or the antisense strand) of the DNA molecule, comprises between 1-100% chemically modified nucleotides, between 1%-90% chemically modified nucleotides, between 1%-80% chemically modified nucleotides, between 1%-70% chemically modified nucleotides, between 1%-60% chemically modified nucleotides, between 1%-50% chemically modified nucleotides, between 1%-40% chemically modified nucleotides, between 1%-30% chemically modified nucleotides, between 1%-20% chemically modified nucleotides, between 1%-15% chemically modified nucleotides, between 1%-10% chemically modified nucleotides, between 20%-90% chemically modified nucleotides, or between 20%-80% chemically modified nucleotides. In embodiments, a DNA molecule described herein, or one strand (e.g., the sense strand or the antisense strand) of the DNA molecule, comprises at least 1% chemically modified nucleotides, at least 5% chemically modified nucleotides; at least 10% chemically modified nucleotides; at least 15% chemically modified nucleotides; at least 20% chemically modified nucleotides; at least 25% chemically modified nucleotides; at least 30% chemically modified nucleotides; at least 40% chemically modified nucleotides; at least 50% chemically modified nucleotides; at least 60% chemically modified nucleotides; at least 70% chemically modified nucleotides; at least 80% chemically modified nucleotides; at least 85% chemically modified nucleotides; at least 90% chemically modified nucleotides; at least 92% chemically modified nucleotides; at least 95% chemically modified nucleotides; or at least 97% chemically modified nucleotides. In embodiments, a DNA molecule described herein, or one strand (e.g., the sense strand or the antisense strand) of the DNA molecule, comprises chemically modified nucleotides at between 0%-100% of each distinct nucleotide, e.g., 0%-100% chemically modified T nucleotides, 0%-100% chemically modified A nucleotides, 0%-100% chemically modified C nucleotides, and 0%-100% chemically modified G nucleotides for each construct. In embodiments, a DNA molecule described herein, or one strand (e.g., the sense strand or the antisense strand) of the double-stranded DNA molecule, comprises chemically modified nucleotides at between 0-100%, 10%-100%, 20%-100%, 30%-100%, 40%-100%, 50%-100%, 60%-100%, 10%-50% of each distinct nucleotide, e.g., between 0-100%, 10%-100%, 20%-100%, 30%-100%, 40%-100%, 50%-100%, 60%-100%, 10%-50% of chemically modified T nucleotides; between 0-100%, 10%-100%, 20%-100%, 30%-100%, 40%-100%, 50%-100%, 60%-100%, 10%-50% of chemically modified A nucleotides; between 0-100%, 10%-100%, 20%-100%, 30%-100%, 40%-100%, 50%-100%, 60%-100%, 10%-50% of chemically modified C nucleotides; or between 0-100%, 10%-100%, 20%-100%, 30%-100%, 40%-100%, 50%-100%, 60%-100%, 10%-50% of chemically modified G nucleotides. For example, a DNA molecule could contain 100% chemically modified T nucleotides, 50% chemically modified A nucleotides, 0% chemically modified C nucleotides, and 25% chemically modified G nucleotides.

In embodiments, chemically modified nucleotides, e.g., modifications described herein, can be introduced in the DNA molecules described herein throughout the entire sequence; within an element of a sequence, e.g., an element described herein; and/or at a 5′- or 3′-end.

In some embodiments, a double-stranded DNA molecule as described herein comprises chemically modified nucleotides on only one strand. In some embodiments, a double-stranded DNA molecule as described herein comprises chemically modified nucleotides on the antisense strand. In some embodiments, a double-stranded DNA molecule as described herein comprises chemically modified nucleotides on the sense strand.

In some embodiments, a double-stranded DNA molecule as described herein comprises chemically modified nucleotides on both strands. In certain embodiments, both strands comprise chemical modifications at the same positions (e.g., chemically modified nucleotides on one strand are base-paired with chemically modified nucleotides on the opposite strand, and/or non-chemically modified nucleotides on one strand are base-paired with non-chemically modified nucleotides on the opposite strand). In embodiments, the entirety of both strands are composed of chemically modified nucleotides. In other embodiments, the two strands of a double-stranded DNA molecule as described herein comprise different chemical modification patterns (e.g., one or more chemically modified nucleotides on one strand are base-paired with non-chemically modified nucleotides on the other strand). In embodiments, a double-stranded DNA molecule as described herein comprises one or more double-stranded regions in which both strands are chemically modified, and/or one or more double-stranded regions in which neither strand is chemically modified. In embodiments, a double-stranded DNA molecule as described herein comprises one or more double-stranded regions in which one strand is chemically modified and the other is not.

In some embodiments, a chemically modified DNA molecule described herein exhibits decreased recognition by DNA sensors in a host tissue or subject compared to an unmodified DNA molecule of the same sequence, e.g., at least 10%, at least 20%, at least 30%, at least 40%, at least 50%, at least 60%, at least 70%, at least 80%, at least 90%, at least 95% or more decreased recognition by DNA sensors in a host tissue or subject compared to an unmodified DNA molecule of the same sequence. In some embodiments, a chemically modified DNA molecule described herein exhibits decreased degradation by DNA nucleases compared to an unmodified DNA molecule of the same sequence, e.g., at least 10%, at least 20%, at least 30%, at least 40%, at least 50%, at least 60%, at least 70%, at least 80%, at least 90%, at least 95% or more decreased degradation by DNA nucleases in a host tissue or subject compared to an unmodified DNA molecule. In some embodiments, a chemically modified DNA molecule described herein shows decreased activation of the innate immune system in a target/host tissue or subject compared to an unmodified DNA molecule of the same sequence, e.g., at least 10%, at least 20%, at least 30%, at least 40%, at least 50%, at least 60%, at least 70%, at least 80%, at least 90%, at least 95% or more decreased activation of the innate immune system in a target/host tissue or subject compared to an unmodified DNA molecule of the same sequence.

In some embodiments, a DNA molecule comprising chemically modified nucleotides described herein exhibits any of the following properties in a target/host tissue or subject compared to a DNA molecule of the same sequence that does not comprise chemically modified nucleotides (unmodified DNA molecule): increased integration of exogenous construct in genome of target cell; increased retention in a target cell through replication; reduced secondary or tertiary structure formation; reduced interaction with innate immune sensors; reduced interaction with nucleases; enhanced stability; enhanced longevity; reduced toxicity; enhanced delivery; increased expression; increased transport across membranes; increased binding to DNA binding moieties such as nuclear DNA binding proteins, transcription factors, chaperones, DNA polymerases. In embodiments, any of the above listed properties is modulated at least 10%, at least 20%, at least 30%, at least 40%, at least 50%, at least 60%, at least 70%, at least 80%, at least 90%, at least 95% or more in a target/host tissue or subject compared to an unmodified DNA molecule of the same sequence.

A nucleobase comprising 5-hydroxymethyluracil is shown below as Formula I.

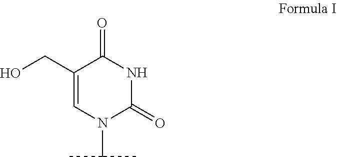

A nucleobase comprising a canonical uracil nucleobase is shown below as Formula II.

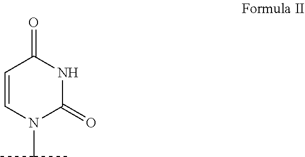

In some embodiments, a dsDNA molecule described herein comprises a chemically modified cytosine nucleobase. In some embodiments, the chemically modified cytosine nucleobase comprises a substitution other than hydrogen at the carbon 5 (C-5) position of the nucleobase. In some embodiments, the chemically modified cytosine nucleobase comprises the structure of Formula III:

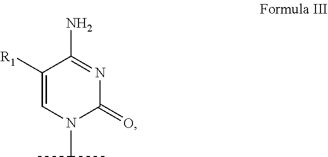

wherein R¹ is selected from the group consisting of —OH; -aldehyde; -carboxylic acid; -alkyl; —(CH₂)ₘOR₂, m=1-3 and R₂═H or a sugar molecule; and -propargylamino. In some embodiments, R¹ is selected from the group consisting of —OH; —CHO; —COOH; -alkyl; —(CH₂)ₘOR₂, m=1-3 and R₂═H or a sugar molecule; and-propargylamino, wherein the alkyl group includes one to six carbons. In some embodiments, R¹ is selected from the group consisting of —OH; —CHO; —COOH; —CH₂OR₃, R₃═H or glucose; -methyl; and -propargylamino. In some embodiments, the chemically modified cytosine nucleobase comprises 5-formylcytosine, 5-hydroxycytosine, 5-carboxycytosine, 5-propargylaminocytosine, 5-methylcytosine, 5-hydroxymethylcytosine, or glucosyl-5-hydroxymethylcytosine. Chemically modified cytosine nucleobases are further described in International Application WO/2024/173836, which is herein incorporated by reference in its entirety.

In some embodiments, a dsDNA molecule described herein comprises a chemically modified uracil nucleobase. In some embodiments, the chemically modified uracil nucleobase comprises N1-methylpseudouracil. In some embodiments, the chemically modified uracil nucleobase comprises a substitution other than hydrogen or a methyl group at the carbon 5 (C-5) position of the nucleobase. In some embodiments, the chemically modified uracil nucleobase comprises the structure of Formula IV:

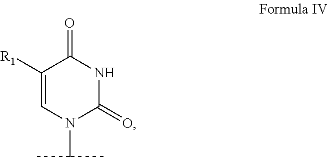

wherein R₁ is selected from the group consisting of —(CH₂)ₘOH, m=1-10; -halogen; —(CH₂)ₙ—CHO, n=0-10; —(CH₂)ₚCOOH, p=0-10; -aminoallyl; —S—(C₁-C₆)alkyl; and -propargylamino. In some embodiments, R₁ is selected from the group consisting of —(CH₂)ₘOH, m=1-6; -halogen; —(CH₂)ₙ—CHO, n=0-6; —(CH₂)ₚCOOH, p=0-6; -aminoallyl; —S—(C1-C3)alkyl; and -propargylamino. In some embodiments, R¹ is selected from the group consisting of —(CH₂) OH; —I; —Br; —CHO; —COOH; -aminoallyl; —S-methyl; and -propargylamino. In some embodiments, the chemically modified uracil nucleobase comprises 5-hydroxymethyluracil, 5-aminoallyluracil, 5-bromouracil, 5-iodouracil, 5-propargylaminouracil, 5-formyluracil, 5-carboxyuracil, 5-methylthiouracil, or 5-dihydroxypentyluracil. Chemically modified uracil nucleobases are further described in International Application WO/2024/173828, which is herein incorporated by reference in its entirety.

In some embodiments, a dsDNA molecule described herein comprises a first type of chemically modified nucleobase and a second type of chemically modified nucleobase. In some embodiments, the first type of chemically modified nucleobase is a chemically modified cytosine nucleobase and the second type of chemically modified nucleobase is a chemically modified uracil nucleobase. In some embodiments, the first type of chemically modified nucleobase is a chemically modified cytosine nucleobase and the second type of chemically modified nucleobase is a different chemically modified cytosine nucleobase. In some embodiments, the first type of chemically modified nucleobase is a chemically modified uracil nucleobase and the second type of chemically modified nucleobase is a different chemically modified uracil nucleobase.

### Asymmetrically Modified Circular DNA Molecules

In some embodiments, a tLNP composition described herein comprises a double stranded DNA molecule (dsDNA molecule), wherein the dsDNA molecule is circular, and the dsDNA molecule comprises a first strand (e.g., sense strand) and a second strand (e.g., an antisense strand), wherein the first strand comprises one or more chemically modified nucleobases, and the second strand is substantially free of (e.g., is free of) chemically modified nucleobases. In some embodiments, the first strand is a sense strand and the second strand is an antisense strand. In some embodiments, the first strand is an antisense strand and the second strand is a sense strand.

In some embodiments, the dsDNA molecule comprises a promoter sequence and an effector sequence that encodes an effector (e.g., a therapeutic effector). In some embodiments, the dsDNA molecule comprises one or more sequences encoding a 5′ untranslated region (5′ UTR) that is 5′ of the effector sequence and/or a 3′ untranslated region (3′ UTR) that is 3′ of the effector sequence. In some embodiments, the dsDNA molecule comprises a sequence encoding a polyadenylation site. In some embodiments, the dsDNA molecule comprises an intron sequence. In some embodiments, the dsDNA molecule comprises an enhancer sequence. In some aspects, one or more regions of a dsDNA molecule described herein comprises a first strand and a second strand, wherein the first strand of said one or more regions comprises one or more chemically modified nucleobases, and the second strand of said one or more regions is substantially free of (e.g., is free of) chemically modified nucleobases. In some embodiments, the dsDNA molecule comprises two or more such regions. In some embodiments, the first strand of the first region is contiguous with the first strand of the second region. In some embodiments, the first strand of the first region is contiguous with the second strand of the second region. In some embodiments, the entire length of the dsDNA molecule is made up of such regions.

In some embodiments, the first strand (e.g., sense strand) comprises one or more backbone modifications, e.g., phosphorothioate linkages. In some embodiments, the second strand (e.g., antisense strand) is substantially free of (e.g., is free of) phosphorothioate linkages. In some embodiments, the second strand (e.g., antisense strand) is substantially free of (e.g., is free of) backbone modifications.

In some embodiments, at least 1%, at least 5%, at least 10%, at least 15%, at least 20%, at least 25%, at least 30%, at least 35%, at least 40%, at least 45%, or at least 50% positions in the first strand (e.g., sense strand) of the dsDNA molecule comprise chemically modified nucleobases. In some embodiments, at least 1%, at least 5%, at least 10%, at least 15%, at least 20%, or at least 25% positions in the first strand (e.g., sense strand) of the dsDNA molecule comprise the same chemically modified nucleobase. In some embodiments, 1%-50% (e.g., 1%-25%, 1%-5%, 5%-10%, 10%-15%, 15%-20%, 20%-25%, 25%-30%, 30%-35%, 35%-40%, 40%-45%, or 45%-50%) positions in the first strand (e.g., sense strand) of the dsDNA molecule comprise chemically modified nucleobases. In some embodiments, 1%-25% (e.g., 1%-5%, 5%-10%, 10%-15%, 15%-20%, or 20%-25%) positions in the first strand (e.g., sense strand) of the dsDNA molecule comprise the same chemically modified nucleobase. In some embodiments, at least 80%, at least 85%, at least 90%, at least 95%, or all the chemically modified nucleobases of the dsDNA molecule have the same chemical structure. In some embodiments, 80%-85%, 85%-90%, 90%-95%, or 95%-100% of the chemically modified nucleobases of the dsDNA molecule have the same chemical structure.

In some embodiments, the chemically modified nucleobase comprises a uracil nucleobase. In some embodiments, the uracil nucleobase is a canonical uracil nucleobase or a chemically modified uracil nucleobase. In some embodiments, the uracil nucleobase is a canonical uracil nucleobase. In some embodiments, the uracil nucleobase is a chemically modified uracil nucleobase. In some embodiments, at least 1%, at least 5%, at least 10%, at least 15%, at least 20%, at least 25%, at least 50%, at least 75%, at least 90%, at least 95%, at least 96%, at least 97%, at least 98%, or at least 99% of thymine or uracil positions in the first strand (e.g., sense strand) of the dsDNA molecule comprise a uracil nucleobase. In some embodiments, 1%-100% (e.g., 1%-5%, 5%-10%, 10%-15%, 15%-20%, 20%-25%, 25%-30%, 30%-35%, 35%-40%, 40%-45%, 45%-50%, 50%-55%, 55%-60%, 60%-65%, 65%-70%, 70%-75%, 75%-80%, 80%-85%, 85%-90%, 90%-95%, 95%-96%, 96%-97%, 97%-98%, 98%-99%, or 95%-100%) of thymine or uracil positions in the first strand (e.g., sense strand) of the dsDNA molecule comprise a uracil nucleobase. In some embodiments, at least 20% of thymine or uracil positions in the first strand (e.g., sense strand) of the dsDNA molecule comprise a uracil nucleobase. In some embodiments, at least 40% of thymine or uracil positions in the first strand (e.g., sense strand) of the dsDNA molecule comprise a uracil nucleobase. In some embodiments, at least 80% of thymine or uracil positions in the first strand (e.g., sense strand) of the dsDNA molecule comprise a uracil nucleobase. In some embodiments, every thymine or uracil position in a stretch of at least 100, at least 200, at least 300, at least 400, at least 500, at least 600, at least 700, at least 800, at least 900, at least 1000, at least 1500, or at least 2000 nucleotides in the first strand (e.g., sense strand) of the dsDNA molecule comprises a uracil nucleobase. In some embodiments, every thymine or uracil position in a stretch of 100-200, 200-300, 300-400, 400-500, 500-600, 600-700, 700-800, 800-900, 900-1000, 1000-1500, or 1500-2000 nucleotides in the first strand (e.g., sense strand) of the dsDNA molecule comprises a uracil nucleobase.

In some embodiments, the chemically modified nucleobase comprises a chemically modified uracil nucleobase. In some embodiments, the chemically modified uracil nucleobase comprises 5-hydroxymethyluracil. In some embodiments, at least 80%, at least 85%, at least 90%, at least 95%, or all of the chemically modified nucleobases of the dsDNA molecule comprise 5-hydroxymethyluracil. In some embodiments, 80%-85%, 85%-90%, 90%-95%, or 95%-100% of the chemically modified nucleobases of the dsDNA molecule comprise 5-hydroxymethyluracil.

In some embodiments, the chemically modified nucleobase comprises a canonical uracil nucleobase. In some embodiments, at least 80%, at least 85%, at least 90%, at least 95%, or all of the chemically modified nucleobases of the dsDNA molecule comprise a canonical uracil nucleobase. In some embodiments, 80%-85%, 85%-90%, 90%-95%, or 95%-100% of the chemically modified nucleobases of the dsDNA molecule comprise a canonical uracil nucleobase.

In some embodiments, the chemically modified nucleobase comprises a chemically modified cytosine nucleobase. In some embodiments, at least 1%, at least 5%, at least 10%, at least 15%, at least 20%, at least 25%, at least 50%, at least 75%, at least 90%, at least 95%, at least 96%, at least 97%, at least 98%, at least 99%, or all of cytosine positions in the first strand (e.g., sense strand) of the dsDNA molecule comprise a chemically modified cytosine nucleobase.

In some embodiments, 1%-100% (e.g., 1%-5%, 5%-10%, 10%-15%, 15%-20%, 20%-25%, 25%-30%, 30%-35%, 35%-40%, 40%-45%, 45%-50%, 50%-55%, 55%-60%, 60%-65%, 65%-70%, 70%-75%, 75%-80%, 80%-85%, 85%-90%, 90%-95%, 95%-96%, 96%-97%, 97%-98%, 98%-99%, 99%-100%, or 95%-100%) of cytosine positions in the first strand (e.g., sense strand) of the dsDNA molecule comprise a chemically modified cytosine nucleobase. In some embodiments, at least 20% of cytosine positions in the first strand (e.g., sense strand) of the dsDNA molecule comprise a chemically modified cytosine nucleobase. In some embodiments, at least 40% of cytosine positions in the first strand (e.g., sense strand) of the dsDNA molecule comprise a chemically modified cytosine nucleobase. In some embodiments, at least 80% of cytosine positions in the first strand (e.g., sense strand) of the dsDNA molecule comprise a chemically modified cytosine nucleobase. In some embodiments, every cytosine position in a stretch of at least 100, at least 200, at least 300, at least 400, at least 500, at least 600, at least 700, at least 800, at least 900, at least 1000, at least 1500, or at least 2000 nucleotides in the first strand (e.g., sense strand) of the dsDNA molecule comprises a chemically modified cytosine nucleobase. In some embodiments, every cytosine position in a stretch of 100-200, 200-300, 300-400, 400-500, 500-600, 600-700, 700-800, 800-900, 900-1000, 1000-1500, or 1500-2000 nucleotides in the first strand (e.g., sense strand) of the dsDNA molecule comprises a chemically modified cytosine nucleobase. In some embodiments, the chemically modified cytosine nucleobase comprises 5-hydroxycytosine.

In some embodiments, the chemically modified nucleobase comprises a chemically modified guanine nucleobase. In some embodiments, at least 1%, at least 5%, at least 10%, at least 15%, at least 20%, at least 25%, at least 50%, or at least 75% of guanine positions in the first strand (e.g., sense strand) of the dsDNA molecule comprise a chemically modified guanine nucleobase. In some embodiments, 1%-75% (e.g., 1%-5%, 5%-10%, 10%-15%, 15%-20%, 20%-25%, 25%-30%, 30%-35%, 35%-40%, 40%-45%, 45%-50%, 50%-55%, 55%-60%, 60%-65%, 65%-70%, or 70%-75%) of guanine positions in the first strand (e.g., sense strand) of the dsDNA molecule comprise a chemically modified guanine nucleobase. In some embodiments, at least 40% of guanine positions in the first strand (e.g., sense strand) of the dsDNA molecule comprise a chemically modified guanine nucleobase. In some embodiments, every guanine position in a stretch of at least 100, at least 200, at least 300, at least 400, at least 500, at least 600, at least 700, at least 800, at least 900, at least 1000, at least 1500, or at least 2000 nucleotides in the first strand (e.g., sense strand) of the dsDNA molecule comprises a chemically modified guanine nucleobase. In some embodiments, every guanine position in a stretch of 100-200, 200-300, 300-400, 400-500, 500-600, 600-700, 700-800, 800-900, 900-1000, 1000-1500, or 1500-2000 nucleotides in the first strand (e.g., sense strand) of the dsDNA molecule comprises a chemically modified guanine nucleobase. In some embodiments, the chemically modified guanine nucleobase comprises inosine.

In some embodiments, the first strand of the dsDNA molecule comprises a first type of chemically modified nucleobase and a second type of chemically modified nucleobase. In some embodiments, the first type of chemically modified nucleobase is a chemically modified cytosine nucleobase, and the second type of chemically modified nucleobase is a different chemically modified cytosine nucleobase, a uracil nucleobase, or a chemically modified guanine nucleobase. In some embodiments, the first type of chemically modified nucleobase is a uracil nucleobase, and the second type of chemically modified nucleobase is a chemically modified cytosine nucleobase, a different uracil nucleobase, or a chemically modified guanine nucleobase. In some embodiments, the first type of chemically modified nucleobase is a chemically modified guanine nucleobase, and the second type of chemically modified nucleobase is a chemically modified cytosine nucleobase or a uracil nucleobase. In some embodiments, the chemically modified guanine nucleobase comprises inosine. In some embodiments, the uracil nucleobase comprises a canonical uracil nucleobase. In some embodiments, the uracil nucleobase comprises 5-hydroxymethyluracil. In some embodiments, the chemically modified cytosine nucleobase comprises 5-hydroxycytosine.

Chemically modified cytosine nucleobases are further described in International Application WO/2024/173836, which is herein incorporated by reference in its entirety. Chemically modified uracil nucleobases are further described in International Application WO/2024/173828, which is herein incorporated by reference in its entirety.

In some embodiments, the longest stretch of unmodified nucleotides in the first strand (e.g., sense strand) is no more than 1000, no more than 900, no more than 800, no more than 700, no more than 600, no more than 500, no more than 400, no more than 300, no more than 200, no more than 100, no more than 50, or no more than 10 nucleotides. In some embodiments, the longest stretch of unmodified nucleotides in the first strand (e.g., sense strand) is 10-50, 50-100, 100-200, 200-300, 300-400, 400-500, 500-600, 600-700, 700-800, 800-900, or 900-1000 nucleotides. In some embodiments, the longest stretch of unmodified nucleobases in the first strand (e.g., sense strand) is no more than 1000, no more than 900, no more than 800, no more than 700, no more than 600, no more than 500, no more than 400, no more than 300, no more than 200, no more than 100, no more than 50, or no more than 10 nucleobases. In some embodiments, the longest stretch of unmodified nucleobases in the first strand (e.g., sense strand) is 10-50, 50-100, 100-200, 200-300, 300-400, 400-500, 500-600, 600-700, 700-800, 800-900, or 900-1000 nucleobases. In some embodiments, the second strand (e.g., antisense strand) comprises no more than 10, no more than 9, no more than 8, no more than 7, no more than 6, no more than 5, no more than 4, no more than 3, no more than 2, or no more than 1 chemically modified nucleotides. In some embodiments, the second strand (e.g., antisense strand) comprises no more than 10, no more than 9, no more than 8, no more than 7, no more than 6, no more than 5, no more than 4, no more than 3, no more than 2, or no more than 1 chemically modified nucleobases. In some embodiments, the second strand (e.g., antisense strand) comprises no more than 10, no more than 9, no more than 8, no more than 7, no more than 6, no more than 5, no more than 4, no more than 3, no more than 2, or no more than 1 backbone modifications. In some embodiments, the second strand (e.g., antisense strand) comprises no more than 10, no more than 9, no more than 8, no more than 7, no more than 6, no more than 5, no more than 4, no more than 3, no more than 2, or no more than 1 nucleotides having a chemically modified sugar.

In some embodiments, the dsDNA molecule further comprises one or more additional chemically modified nucleotide, wherein the additional chemically modified nucleotide comprises a modification in the backbone, sugar, or nucleobase. In some embodiments, one or more of the chemically modified nucleotides is conjugated to a peptide or protein. In some embodiments, one or more of the chemically modified nucleotides comprises a phosphorothioate linkage. In some embodiments, each of the first strand (e.g., sense strand) and second strand (e.g., antisense strand) of the dsDNA molecule comprises one or more chemically modified nucleotides. In some embodiments, each of the first strand (e.g., sense strand) and second strand (e.g., antisense strand) of the dsDNA molecule comprises one or more phosphorothioate linkages.

In some aspects, the present disclosure provides a composition, e.g., a pharmaceutical composition, comprising the dsDNA molecule. In some aspects, the present disclosure provides a composition, e.g., a pharmaceutical composition, comprising a plurality of the dsDNA molecules.

Chemically modified DNA molecules, compositions comprising said DNA molecules, methods of making and using such compositions are further described in International Application WO/2026/006577, which is herein incorporated by reference in its entirety.

### CpG Depleted DNA Sequences

A double-stranded DNA molecule described herein may comprise a sequence that is CpG-depleted. A CpG of a dsDNA molecule refers to a dinucleotide region of the DNA molecule where a cytosine nucleotide is directly followed by a guanine nucleotide in the linear sequence of bases along the 5′ to 3′ direction. The “P” represents the phosphate group between the C and G. Without wishing to be bound by theory, the depletion of CpG's from a dsDNA molecule is thought to lead to a weakened immune response against the dsDNA molecule in a subject administered the CpG-depleted dsDNA. For instance, CpG depletion may lead to lower activation of IL-6 and/or IP-10. No particular method of making is implied in the term “CpG depleted”. For instance, a CpG-depleted dsDNA may be synthesized de novo.

Various CpG depleted molecules are provided herein. A dsDNA molecule described herein may be substantially free of (e.g., free of) CpG's. In some embodiments, the portion of the nucleotide sequence that is substantially free of (e.g., free of) CpG's is at least 10%, at least 20%, at least 30%, at least 40%, at least 50%, at least 60%, at least 70%, at least 80%, at least 85%, at least 90%, at least 95%, at least 99%, or 100% of the nucleotide sequence of the dsDNA molecule. In some embodiments, the portion of the nucleotide sequence that is substantially free of (e.g., free of) CpG's is between 10% and 20%, between 20% and 30%, between 30% and 40%, between 40% and 50%, between 50% and 60%, between 60% and 70%, between 70% and 80%, between 80% and 85%, between 85% and 90%, between 90% and 95%, between 95% and 99%, or between 99% and 100% of the nucleotide sequence of the dsDNA molecule. In some embodiments, a nucleotide sequence of a dsDNA molecule described herein comprises no more than 1, no more than 5, no more than 10, no more than 20, or no more than 50 CpG's. In some embodiments, a nucleotide sequence of a dsDNA molecule described herein comprises between 0 and 5, between 5 and 10, between 10 and 20, or between 20 and 50 CpG's. In some embodiments, a nucleotide sequence of a dsDNA molecule described herein does not comprise any CpG's.

In some embodiments, a nucleotide sequence of interest may have fewer CpG's in comparison to a reference sequence. In some embodiments, the reference sequence has at least 40%, at least 50%, at least 60%, at least 70%, at least 80%, or at least 90% sequence identity to the CpG-depleted nucleotide sequence. The reference sequence and the CpG-depleted nucleotide sequence may each comprise: an effector sequence (e.g., an effector sequence that encodes the same effector as the nucleotide sequence), a promoter sequence, an exon sequence, an intron sequence, a sequence encoding a 5′ untranslated region, a sequence encoding a 3′ untranslated region, an enhancer sequence, a sequence encoding a polyadenylation site, or any combination thereof. In some embodiments, at least 10%, at least 20%, at least 30%, at least 40%, at least 50%, at least 60%, at least 70%, at least 80%, at least 90%, at least 95%, at least 99%, or 100% of CpG's that are present in a reference sequence are absent in the CpG-depleted nucleotide sequence. In some embodiments, the effector is a naturally occurring effector. In other embodiments, the effector is not a naturally occurring effector. In some embodiments, a CpG-depleted nucleotide sequence is a non-coding sequence.

A CpG-depleted nucleotide sequence may be manually designed by a user or may be designed computationally. When designing a CpG-depleted nucleotide sequence that comprises an effector sequence encoding an effector, a user may remove one or more (e.g., all) CpG's present in a reference sequence comprising an effector sequence that encodes the same effector. The CpG-depleted nucleotide sequence may be designed, for example, using the DNA Chisel program, described in Zulkower et al., Bioinformatics, 36(16): 4508-4509, 2020, doi.org/10.1093/bioinformatics/btaa558, which is hereby incorporated by reference in its entirety. For example, for coding regions, in some embodiments, the user begins with a reference nucleotide sequence and only changes codons that are directly affected by a CpG, e.g., by changing the codon comprising a CpG to a codon that encodes the same amino acid but lacks a CpG. Alternatively, a user may alter the sequence to remove CpG's in combination with a codon optimization strategy. In some embodiments, the codon optimization strategy is based on the codon usage for a set of reference genes. In some embodiments, the reference genes are general human genes. In some embodiments, the reference genes are T-cell specific genes. The CpG may be changed by substituting the C and/or the G.

CpG depletion can also be performed on a noncoding region. For instance, in some embodiments, one or more (e.g., substantially all, e.g., all) of the CpG's in a reference nucleotide sequence are converted to a CpA sequence to prepare the CpG-depleted nucleotide sequence. In some embodiments, one or more (e.g., substantially all, e.g., all) of the CpG's in a reference nucleotide sequence are converted to a randomized dinucleotide that is not a CpG. In some embodiments, one or more (e.g., substantially all, e.g., all) of the CpG's in a reference nucleotide sequence are converted to a dinucleotide that is not a CpG, in a way designed to minimize the impact on potential transcription factor binding sites. In some embodiments, one or more (e.g., substantially all, e.g., all) of the CpG is converted to a dinucleotide that is not a CpG, through the use of a model (e.g., a machine learning model). In some embodiments, the model is used to predict function from the nucleotide sequence. In some embodiments, the model is used to preserve one or more of the functional properties of the nucleotide sequence while simultaneously removing one or more (e.g., all) of the CpG's from the reference sequence. In some embodiments, the model used is Borzoi, as described in Linder et al., Nat Genet 57: 949-961, 2025, doi.org/10.1038/s41588-024-02053-6, which is hereby incorporated by reference in its entirety. In some embodiments, the model used is dhs733, as described in Castillo-Hair et al., biorxiv, 2025, doi.org/10.1101/2025.09.30.679565, which is hereby incorporated by reference in its entirety. In some embodiments, the model used is a machine learning model trained on genetic data (e.g., publicly available Massively Parallel Reporter Assay (MPRA) data) to predict gene expression (e.g., cell-type-specific gene expression) from a nucleotide sequence.

### Chimeric Antigen Receptor (CAR)

In some embodiments, a DNA molecule described herein comprises an effector sequence that encodes a chimeric antigen receptor (CAR). In some embodiments, the CAR is a first generation CAR. In some embodiments, the first generation CAR comprises an antigen binding domain, a transmembrane domain, and a primary signaling domain. In some embodiments, the primary signaling domain mediates downstream signaling during T-cell activation.

In some embodiments, a DNA molecule described herein comprises an effector sequence that encodes a second generation CAR. In some embodiments, the second generation CAR comprises an antigen binding domain, a transmembrane domain, and two signaling domains. In some embodiments, one of said signaling domains is a primary signaling domain that mediates downstream signaling during T-cell activation. In some embodiments, the second of said signaling domains is a costimulatory domain. In some embodiments, the costimulatory domain enhances cytokine production, CAR T-cell proliferation, and or CAR T-cell persistence during T cell activation.

In some embodiments, a DNA molecule described herein comprises an effector sequence that encodes a third generation CAR. In some embodiments, the third generation CAR comprises an antigen binding domain, a transmembrane domain, and at least three signaling domains. In some embodiments, one of said signaling domains is a primary signaling domain that mediates downstream signaling during T-cell activation. In some embodiments, the second of said signaling domains is a costimulatory domain. In some embodiments, the costimulatory domain enhances cytokine production, CAR T-cell proliferation, and or CAR T-cell persistence during T cell activation. In some embodiments, a third generation CAR comprises at least two costimulatory domains. In some embodiments, the at least two costimulatory domains are not the same.

In some embodiments, a DNA molecule described herein comprises an effector sequence that encodes a fourth generation CAR. In some embodiments, the fourth generation CAR comprises an antigen binding domain, a transmembrane domain, and at least two, three, or four signaling domains. In some embodiments, one of said signaling domains is a primary signaling domain that mediates downstream signaling during T-cell activation. In some embodiments, one or more of said signaling domains is a costimulatory domain. In some embodiments, the costimulatory domain enhances cytokine production, CAR T-cell proliferation, and or CAR T-cell persistence during T cell activation.

In some embodiments, a DNA molecule comprising an effector sequence that encodes a CAR is delivered to a cell, e.g., a T cell. In some embodiments, the T cell is a mammalian T cell, e.g., a human T cell.

### CAR Antigen Binding Domains

In some embodiments, a CAR antigen binding domain described herein is or comprises an antibody or antigen-binding portion thereof. In some embodiments, a CAR antigen binding domain described herein is or comprises an scFv or Fab. In some embodiments a CAR antigen binding domain described herein comprises an scFv or Fab fragment of an anti-CD19 antibody, an anti-BCMA antibody, an anti-CD20 antibody, an anti-CD22 antibody, an anti-B cell activating factor-receptor (BAFF-R) antibody, an anti-CD30 antibody, an anti-CD5 antibody, an anti-CD7 antibody, or an anti-CD4 antibody.

In some embodiments, a CAR antigen binding domain described herein binds to a cell surface antigen of a cell. In some embodiments, the cell surface antigen is characteristic of one type of cell. In some embodiments, the cell surface antigen is characteristic of more than one type of cell.

In some embodiments, a CAR antigen binding domain described herein binds an antigen characteristic of a cancer. In some embodiments, the antigen binding domain binds a tumor antigen. In some embodiments, the antigen binding domain binds CD19, BCMA, CD20, CD22, BAFF-R, CD30, CD5, CD7, or CD4.

In some embodiments, a CAR antigen binding domain described herein binds an antigen characteristic of an infectious disease (e.g. a viral infection or a bacterial infection).

In some embodiments, a CAR antigen binding domain described herein binds an antigen characteristic of an autoimmune or inflammatory disorder. In some embodiments, the antigen is characteristic of lupus (e.g., systemic lupus erythematosus (SLE)), systemic sclerosis, idiopathic inflammatory myopathy, dermatomyositis, or myasthenia gravis.

### CAR Transmembrane Domains

In some embodiments, a CAR described herein comprises a transmembrane domain. In some embodiments, the CAR comprises at least a transmembrane region of CD8a, CD28, CD3z, CD4, or ICOS, or a functional variant thereof.

### CAR Hinge Domains

In some embodiments, a CAR described herein comprises a hinge region. In some embodiments, a transmembrane domain of a CAR can be attached to an antigen binding domain of the CAR via a hinge region. In some embodiments, the hinge region comprises a CD28 hinge, a CD8α hinge, or an IgG4 hinge.

### CAR Signaling Domains

In some embodiments, a CAR described herein comprises a primary signaling domain of one or more of CD3 zeta, FcγR, or an immunoreceptor tyrosine-based activation motif (ITAM), or a functional variant thereof.

In some embodiments, a CAR described herein comprises a costimulatory domain. In some embodiments a CAR comprises a second costimulatory domain. In some embodiments a CAR comprises at least two costimulatory domains. In some embodiments a CAR comprises at least three costimulatory domains. In some embodiments a CAR comprises a costimulatory domain selected from one or more of 4-1BB (CD137), CD28, ICOS, OX-40, or CD27, or a functional variant thereof.

### CAR Spacers

In some embodiments, a CAR described herein comprises one or more spacers. In some embodiments, a CAR described herein comprises a spacer between the antigen binding domain and the transmembrane domain. In some embodiments, a CAR described herein comprises a spacer between the transmembrane domain and the intracellular signaling domain.

### Production

In some aspects, the present disclosure provides a method of making a composition described herein (e.g., a tLNP composition described herein). In some embodiments, the method comprises contacting a dsDNA molecule described herein, or a population of dsDNA molecules described herein, with a lipid (e.g., an ionizable lipid). In some embodiments, the composition is produced by diluting the dsDNA molecules in a buffer (e.g., a citrate buffer, e.g., a citrate buffer at pH 4.0) to make a dsDNA composition, and diluting a lipid composition (e.g., a lipid composition comprising an ionizable lipid, DSPC, cholesterol, DMG-PEG2000, and DSPE-PEG2000-DBCO at a mol ratio of 50/10/38.5/1/0.5), e.g., in ethanol. In some embodiments, the dsDNA composition and the lipid composition are mixed, e.g., at a ratio of about 3:1 dsDNA composition:lipid composition (vol:vol). In some embodiments, the dsDNA molecules and the lipid composition are mixed at a nitrogen:phosphate (N/P) ratio of 3-9, e.g., 4-8 or 5-7, e.g., about 6. In some embodiments, the method comprises performing a click reaction such that a lipid is linked to a targeting moiety that binds a T cell antigen via a click linker. In some embodiments, the targeting moiety comprises an anti-CD3 antibody. In some embodiments, the anti-CD3 antibody molecule comprises a heavy chain complementarity determining region 1 (CDR1), heavy chain complementarity determining region 2 (CDR2), and heavy chain complementarity determining region 3 (CDR3) of the amino acid sequence of SEQ ID NO: 2, according to the Kabat definition. In some embodiments, the anti-CD3 antibody molecule comprises a light chain CDR1, light chain CDR2, and light chain CDR3 of the amino acid sequence of SEQ ID NO: 3, according to the Kabat definition. In some embodiments, the anti-CD3 antibody molecule comprises a heavy chain variable region comprising the amino acid sequence of SEQ ID NO: 2, or an amino acid sequence with at least 95% identity thereto, and/or a light chain variable region comprising the amino acid sequence of SEQ ID NO: 3, or an amino acid sequence with at least 95% identity thereto.

In some aspects, the present disclosure provides a method that comprises making or manufacturing a DNA molecule, the method comprising (a) providing a DNA molecule described herein, and (b) determining whether the structure of the DNA molecule matches a reference structure, thereby making or manufacturing the DNA molecule. In some embodiments, the determining of (b) comprises sequencing the DNA molecule. In some embodiments, the determining of (b) comprises digesting the DNA molecule with a restriction enzyme. In some embodiments, the structure of the DNA molecule that matches the reference structure is identical to the reference structure. In some embodiments, the structure of the DNA molecule that matches the reference structure has the same sequence as the reference structure. In some embodiments, the structure of the DNA molecule that matches the reference structure has the same length as the reference structure.

A composition (e.g., a tLNP composition) described herein may be enriched or purified from impurities or byproducts selected from the group consisting of: endotoxin, mononucleotides, chemically modified mononucleotides, single stranded DNA, proteins (e.g., enzymes, e.g., ligases, restriction enzymes), or DNA fragments or truncations. In some embodiments, a composition described herein is substantially free of process byproducts and impurities, e.g., process byproducts or impurities described herein.

In some embodiments, a composition described herein, a DNA molecule described herein, is formulated with a lipid-based carrier, e.g., a lipid nanoparticle (LNP).

A DNA molecule described herein may be sequenced to confirm the desired, designed sequence. In embodiments, other structural analysis of the DNA molecule (e.g., restriction enzyme analysis) may be performed to confirm or verify its sequence.

### Enrichment

A composition, e.g., a tLNP composition, described herein is typically enriched to remove process impurities and/or contaminants. In some embodiments, a composition comprising a DNA molecule described herein is enriched. For instance, in some embodiments, at least 60%, at least 65%, at least 70%, at least 75%, or at least 80% by mass of total DNA in the composition may be the DNA molecule. As an example, the composition may also comprise other forms of DNA, e.g., as a process impurity, for instance host cell DNA. As an example, the composition may comprise a contaminant, such as bacterial or viral or fungal agents.

In some embodiments, a composition described herein (e.g., a tLNP composition comprising a DNA molecule, e.g., a pharmaceutical composition comprising a DNA molecule, or a manufacturing intermediate) is free of or is substantially free of one or more process impurity or contaminant, e.g., as described in this section. In some embodiments, a method described herein results in a composition that is free of or is substantially free of one or more process impurity or contaminant, e.g., as described in this section. In some embodiments, a method described herein comprises a step of assaying for one or more process impurity or contaminant, e.g., as described in this section. In some embodiments, the method comprises approving or releasing a batch if the batch is free of or substantially free of the process impurity or contaminant or meets a release criterion for that process impurity or contaminant.

In some embodiments, the process impurity comprises a nonhuman animal serum (e.g., fetal bovine serum); an enzyme, e.g., a ligase, a polymerase, or a digestive enzyme (e.g., a trypsin, a collagenase, a DNase, a RNase, an exonuclease, or an endonuclease, e.g., a restriction endonuclease); a growth factor; a cytokine; an antibody (e.g., a monoclonal antibody); a bead (e.g., an antibody-coated bead); an antibiotic; a cell culture medium; a component of a cell culture medium; a detergent; a protein, e.g., a host cell protein; an extraneous nucleic acid sequence (e.g., a mononucleotide (e.g., a modified mononucleotide), or a DNA fragment or truncation); helper virus contaminant (e.g., infectious virus, viral DNA, or viral proteins); a solvent; a cellular debris; a cell; a pyrogen; a fungus; or any combination thereof, or a portion of any of the foregoing. In some embodiments, the contaminant was a component introduced during a manufacturing process. In some embodiments, the contaminant comprises a viral protein.

In some embodiments, the contaminant comprises an agent for transmissible spongiform encephalopathy (TSE). In some embodiments, a test for this contaminant is performed on a composition for which a bovine material was used in manufacturing.

In some embodiments, the contaminant comprises a zoonotic virus, a porcine circovirus 1, a porcine circovirus 2, or a porcine parvovirus; or any combination thereof, or a portion of any of the foregoing. In some embodiments, a test for this contaminant is performed on a composition for which non-human animal material, e.g., a porcine material, was used in manufacturing.

In some embodiments, the contaminant comprises a virus or portion thereof, e.g., a human virus; human immunodeficiency virus (HIV); HIV-1; HIV-2; hepatitis B virus (HBV); hepatitis C virus (HCV); human TSE, including Creutzfeldt-Jakob disease (CJD); variant CJD (vCJD); Treponema pallidum (syphilis); human T-lymphotropic virus (HTLV), HTLV-1, HTLV-2; or cytomegalovirus, human herpesvirus (e.g., human herpesvirus-6, -7 or -8 (HHV-6, -7, or -8)), JC virus, BK virus, Epstein-Barr virus (EBV), human parvovirus B19, human papillomavirus (HPV); an adenovirus, e.g., adenovirus E1; SV40 Large T antigen sequence; HPV E6 or E7 DNA; or any combination thereof, or a portion of any of the foregoing. In some embodiments, a test for this contaminant is performed on a composition for which human donor cells (e.g., leukocyte-rich cells) were used in manufacturing. In some embodiments, a test for this contaminant is performed on a cell bank.

In some embodiments, the contaminant comprises a microbe or a portion thereof, a bacterium (e.g., a Gram-negative bacterium); mycoplasma; spiroplasma (e.g., when insect cells are used); bacterial toxin (e.g., endotoxin); or an adventitious agent, e.g., an adventitious viral agent or a non-viral adventitious agent, or any combination thereof, or a portion of any of the foregoing. In some embodiments, the contaminant comprises a simian virus, e.g., simian polyomavirus SV40 or simian retrovirus, or any combination thereof, or a portion of any of the foregoing. In some embodiments, the contaminant comprises an arbovirus. In some embodiments, the contaminant comprises a bacteriophage. In some embodiments, a test for this contaminant is performed on a cell bank, e.g., a cell bank of bacterial cells.

In some embodiments, the contaminant or process impurity comprises DNA from a host cell, e.g., wherein the host cell is a non-tumorigenic cell. In some embodiments, the DNA is present at a level of less than 10 ng/dose. In some embodiments, the DNA size is below about 200 nucleotides in length.

In some embodiments, the contaminant is an endotoxin. In some embodiments, a level of the endotoxin is less than 5 Endotoxin Unit (EU)/kg body weight/hour, e.g., wherein the composition is formulated for parenteral administration. In some embodiments, a level of the endotoxin is less than 0.2 EU/kg body weight/hour, e.g., wherein the composition is formulated for intrathecal administration. In some embodiments, a level of the endotoxin is not more than 2.0 EU/dose/eye, e.g., wherein the composition is formulated for injection or implantation into the eye, or not more than 0.5 EU/mL, e.g., wherein the composition is formulated for intraocular administration.

In some embodiments, a process impurity comprises an organic solvent, e.g., an aromatic organic solvent, e.g., phenol or chloroform.

In some embodiments, the contaminant or process impurity is described in Chemistry, Manufacturing, and Control (CMC) Information for Human Gene Therapy Investigational New Drug Applications (INDs)—Guidance for Industry (U.S. Department of Health and Human Services, Food and Drug Administration, Center for Biologics Evaluation and Research, January 2020), which is herein incorporated by reference in its entirety.

In some embodiments, the composition is substantially free of (e.g., is free of) a polymerase.

In some embodiments, the composition is substantially free of (e.g., is free of) agarose. In some embodiments, the composition is substantially free of (e.g., is free of) acrylamide.

In some embodiments, the composition is substantially free of (e.g., is free of) polypeptides.

### Pharmaceutical Compositions

In some aspects, the present disclosure includes a composition, e.g., a tLNP composition or a DNA molecule described herein, in combination with one or more pharmaceutically acceptable excipients and/or carriers.

Pharmaceutical compositions may optionally comprise one or more additional active substances, e.g., therapeutically and/or prophylactically active substances. Pharmaceutical compositions of the present invention are generally sterile and/or pyrogen-free.

Pharmaceutically acceptable excipients or diluents may comprise an inactive substance that serves as a vehicle or medium for the compositions described herein, such as any one of the inactive ingredients approved by the United States Food and Drug Administration (FDA) and listed in the Inactive Ingredient Database, which is incorporated by reference herein. Non-limiting examples of pharmaceutically acceptable excipients or diluents include solvents, aqueous solvents, non-aqueous solvents, tonicity agents, dispersion media, cryoprotectants, diluents, suspension aids, surface active agents, isotonic agents, thickening agents, emulsifying agents, preservatives, hyaluronidases, dispersing agents, preservatives, lubricants, granulating agents, disintegrating agents, binding agents, antioxidants, buffering agents (e.g., phosphate buffered saline (PBS)), lubricating agents, oils, and mixtures thereof.

General considerations in the formulation and/or manufacture of pharmaceutical agents may be found, for example, in Remington: The Science and Practice of Pharmacy 21st ed., Lippincott Williams & Wilkins, 2005 (incorporated herein by reference).

### Carriers

A DNA molecule described herein may also be formulated, or included, with a carrier. General considerations of carriers and delivery of pharmaceutical agents may be found, for example, in “Delivery Technologies for Biopharmaceuticals: Peptides, Proteins, Nucleic Acids and Vaccines” (Lene Jorgensen and Hanne Morck Nielson, Eds.) Wiley; 1st edition (Dec. 21, 2009); and Vargason et al. 2021. Nat Biomed Eng 5, 951-967.

Non-limiting examples of carriers include carbohydrate carriers (e.g., an anhydride-modified phytoglycogen or glycogen-type material, GalNAc), nanoparticles (e.g., a nanoparticle that encapsulates or is covalently linked to the DNA molecule, gold nanoparticles, silica nanoparticles), lipid particles (e.g., liposomes, lipid nanoparticles), cationic carriers (e.g., a cationic lipopolymer or transfection reagent), fusosomes, non-nucleated cells (e.g., ex vivo differentiated reticulocytes), nucleated cells, exosomes, protein carriers (e.g., a protein covalently linked to the DNA molecule), peptides (e.g., cell-penetrating peptides), materials (e.g., graphene oxide), single pure lipids (e.g., cholesterol), DNA origami (e.g., DNA tetrahedron).

In one embodiment, the DNA molecules, compositions, constructs and systems described herein can be formulated in liposomes or other similar vesicles. Liposomes are spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679 for review).

Vesicles can be made from several different types of lipids; however, phospholipids are most commonly used to generate liposomes as drug carriers. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

Exosomes can also be used as drug delivery vehicles for the compositions and systems described herein. For a review, see Ha et al. July 2016. Acta Pharmaceutica Sinica B. Volume 6, Issue 4, Pages 287-296; doi.org/10.1016/j.apsb.2016.02.001.

Ex vivo differentiated red blood cells can also be used as a carrier for an agent (e.g., a DNA molecule) described herein. See, e.g., WO2015073587; WO2017123646; WO2017123644; WO2018102740; WO2016183482; WO2015153102; WO2018151829; WO2018009838; Shi et al. 2014. Proc Natl Acad Sci USA. 111(28): 10131-10136; U.S. Pat. No. 9,644,180; Huang et al. 2017. Nature Communications 8: 423; Shi et al. 2014. Proc Natl Acad Sci USA. 111(28): 10131-10136.

Fusosome compositions, e.g., as described in WO2018208728, can also be used as carriers to deliver the DNA molecules described herein.

### Lipid Nanoformulations Lipid-Based Carriers

In some embodiments, compounds, e.g., DNA molecules, described herein are formulated into a lipid-based carrier (or lipid nanoformulation). In some embodiments, both a DNA molecule and an RNA molecule described herein are formulated into the lipid-based carrier. In some embodiments, the lipid-based carrier (or lipid nanoformulation) is a liposome or a lipid nanoparticle (LNP). In one embodiment, the lipid-based carrier is an LNP.

In some embodiments, the lipid-based carrier (or lipid nanoformulation) comprises a cationic lipid (e.g., an ionizable lipid), a non-cationic lipid (e.g., phospholipid), a structural lipid (e.g., cholesterol), and a PEG-modified lipid. In some embodiments, the lipid-based carrier (or lipid nanoformulation) contains one or more compounds described herein, or a pharmaceutically acceptable salt thereof.

As described herein, suitable compounds to be used in the lipid-based carrier (or lipid nanoformulation) include all the isomers and isotopes of the compounds described above, as well as all the pharmaceutically acceptable salts, solvates, or hydrates thereof, and all crystal forms, crystal form mixtures, and anhydrides or hydrates.

In addition to one or more compounds described herein, the lipid-based carrier (or lipid nanoformulation) may further include a second lipid. In some embodiments, the second lipid is a cationic lipid, a non-cationic (e.g., neutral, anionic, or zwitterionic) lipid, or an ionizable lipid.

One or more naturally occurring and/or synthetic lipid compounds may be used in the preparation of the lipid-based carrier (or lipid nanoformulation).

The lipid-based carrier (or lipid nanoformulation) may contain positively charged (cationic) lipids, neutral lipids, negatively charged (anionic) lipids, or a combination thereof.

### Cationic Lipids (Positively Charged) and Ionizable Lipids

In some embodiments, the lipid-based carrier (or lipid nanoformulation) comprises one or more cationic lipids, e.g., a cationic lipid that can exist in a positively charged or neutral form depending on pH, or an amine-containing lipid that can be readily protonated. In some embodiments, the cationic lipid is a lipid capable of being positively charged, e.g., under physiological conditions.

Exemplary cationic lipids include one or more amine group(s) which bear the positive charge. Examples of positively charged (cationic) lipids include, but are not limited to, N,N′-dimethyl-N,N′-dioctacyl ammonium bromide (DDAB) and chloride DDAC), N-(1-(2,3-dioleyloxy)propyl)-N,N,N-trimethylammonium chloride (DOTMA), 3β-[N—(N′,N′-dimethylaminoethyl)carbamoyl) cholesterol (DC-chol), 1,2-dioleoyloxy-3-[trimethylammonio]-propane (DOTAP), 1,2-dioctadecyloxy-3-[trimethylammonio]-propane (DSTAP), and 1,2-dioleoyloxypropyl-3-dimethyl-hydroxy ethyl ammonium chloride (DORI), N,N-dioleyl-N,N-dimethylammonium chloride (DODAC), N,N-dimethyl-2,3-dioleyloxy)propylamine (DODMA), 1,2-Dioleoyl-3-Dimethylammonium-propane (DODAP), 1,2-Dioleoylcarbamyl-3-Dimethylammonium-propane (DOCDAP), 1,2-Dilineoyl-3-Dimethylammonium-propane (DLINDAP), 3-Dimethylamino-2-(Cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethyl-1-(cis, cis-9′,12′-octadecadienoxy)propane (CpLin DMA), N,N-Dimethyl-3,4-dioleyloxybenzylamine (DMOBA), and the cationic lipids described in e.g. Martin et al., Current Pharmaceutical Design, pages 1-394, which is herein incorporated by reference in its entirety. In some embodiments, the lipid-based carrier (or lipid nanoformulation) comprises more than one cationic lipid.

In some embodiments, the lipid-based carrier (or lipid nanoformulation) comprises a cationic lipid having an effective pKa over 6.0. In some embodiments, the lipid-based carrier (or lipid nanoformulation) further comprises a second cationic lipid having a different effective pKa (e.g., greater than the first effective pKa) than the first cationic lipid.

In some embodiments, cationic lipids that can be used in the lipid-based carrier (or lipid nanoformulation) include, for example those described in Table 4 of WO 2019/217941, which is incorporated by reference.

In some embodiments, the cationic lipid is an ionizable lipid (e.g., a lipid that is protonated at low pH, but that remains neutral at physiological pH). In some embodiments, the lipid-based carrier (or lipid nanoformulation) may comprise one or more additional ionizable lipids, different than the ionizable lipids described herein. Exemplary ionizable lipids include, but are not limited to,

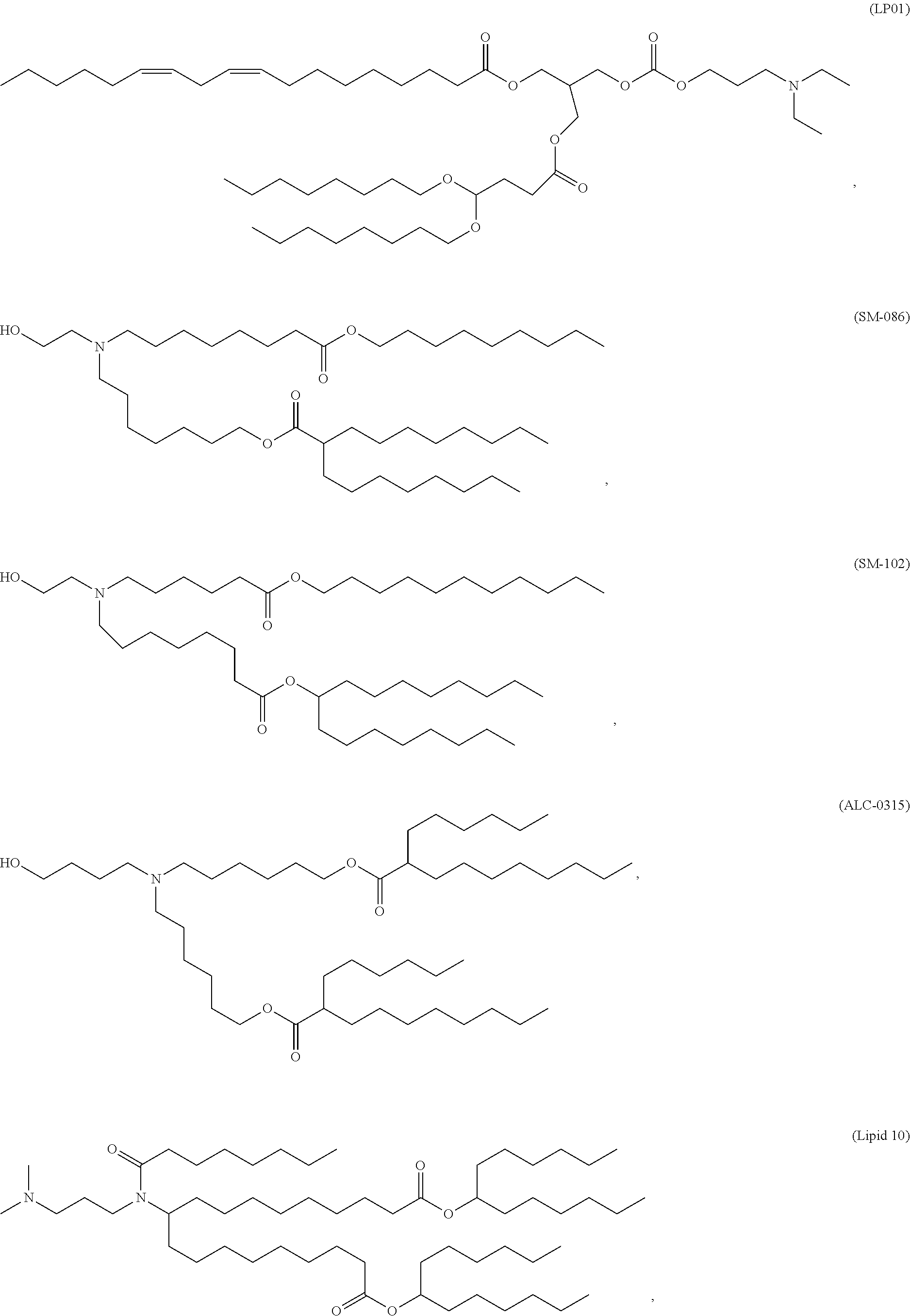

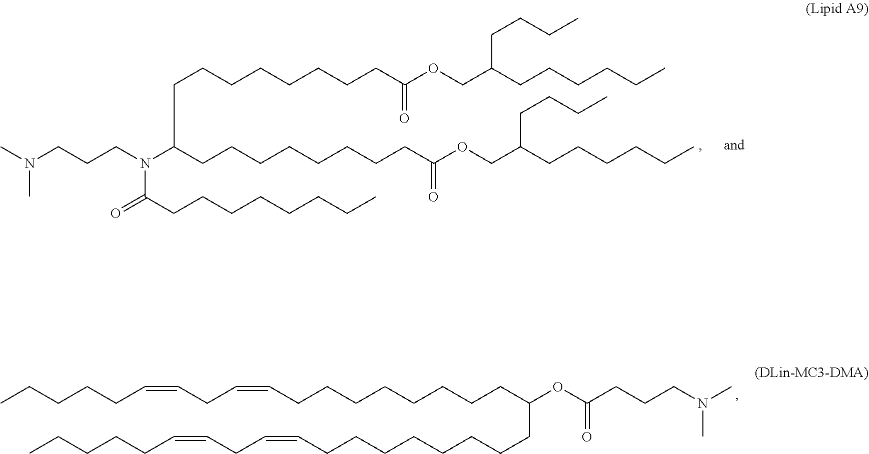

(see WO 2017/004143A1, which is incorporated herein by reference in its entirety).

In some embodiments, the lipid-based carrier (or lipid nanoformulation) further comprises one or more compounds described by WO 2021/113777 (e.g., a lipid of Formula (3) such as a lipid of Table 3 of WO 2021/113777), which is incorporated herein by reference in its entirety.

In one embodiment, the ionizable lipid is a lipid disclosed in Hou, X., et al. Nat Rev Mater 6, 1078-1094 (2021). doi.org/10.1038/s41578-021-00358-0 (e.g., L319, C12-200, and DLin-MC3-DMA), (which is incorporated by reference herein in its entirety).

Examples of other ionizable lipids that can be used in lipid-based carrier (or lipid nanoformulation) include, without limitation, one or more of the following formulas: X of US 2016/0311759; I of US 20150376115 or in US 2016/0376224; Compound 5 or Compound 6 in US 2016/0376224; I, IA, or II of U.S. Pat. No. 9,867,888; I, II or III of US 2016/0151284; I, IA, II, or IIA of US 2017/0210967; I-c of US 2015/0140070; A of US 2013/0178541; I of US 2013/0303587 or US 2013/0123338; I of US 2015/0141678; II, III, IV, or V of US 2015/0239926; I of US 2017/0119904; I or II of WO 2017/117528; A of US 2012/0149894; A of US 2015/0057373; A of WO 2013/116126; A of US 2013/0090372; A of US 2013/0274523; A of US 2013/0274504; A of US 2013/0053572; A of WO 2013/016058; A of WO 2012/162210; I of US 2008/042973; I, II, III, or IV of US 2012/01287670; I or II of US 2014/0200257; I, II, or III of US 2015/0203446; I or III of US 2015/0005363; I, IA, IB, IC, ID, II, IIA, IIB, IIC, IID, or III-XXIV of US 2014/0308304; of US 2013/0338210; I, II, III, or IV of WO 2009/132131; A of US 2012/01011478; I or XXXV of US 2012/0027796; XIV or XVII of US 2012/0058144; of US 2013/0323269; I of US 2011/0117125; I, II, or III of US 2011/0256175; I, II, III, IV, V, VI, VII, VIII, IX, X, XI, XII of US 2012/0202871; I, II, III, IV, V, VI, VII, VIII, X, XII, XIII, XIV, XV, or XVI of US 2011/0076335; I or II of US 2006/008378; I of WO2015/074085 (e.g., ATX-002); I of US 2013/0123338; I or X-A-Y-Z of US 2015/0064242; XVI, XVII, or XVIII of US 2013/0022649; I, II, or III of US 2013/0116307; I, II, or III of US 2013/0116307; I or II of US 2010/0062967; I-X of US 2013/0189351; I of US 2014/0039032; V of US 2018/0028664; I of US 2016/0317458; I of US 2013/0195920; 5, 6, or 10 of U.S. Pat. No. 10,221,127; 111-3 of WO 2018/081480; I-5 or I-8 of WO 2020/081938; I of WO 2015/199952 (e.g., compound 6 or 22) and Table 1 therein; 18 or 25 of U.S. Pat. No. 9,867,888; A of US 2019/0136231; II of WO 2020/219876; 1 of US 2012/0027803; OF-02 of US 2019/0240349; 23 of U.S. Pat. No. 10,086,013; cKK-E12/A6 of Miao et al (2020); C12-200 of WO 2010/053572; 7C1 of Dahlman et al (2017); 304-013 or 503-013 of Whitehead et al; TS-P4C2 of U.S. Pat. No. 9,708,628; I of WO 2020/106946; I of WO 2020/106946; (1), (2), (3), or (4) of WO 2021/113777; and any one of Tables 1-16 of WO 2021/113777, all of which are incorporated herein by reference in their entirety.

In some embodiments, the lipid-based carrier (or lipid nanoformulation) further includes biodegradable ionizable lipids, for instance, (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate). See, e.g., lipids of WO 2019/067992, WO 2017/173054, WO 2015/095340, and WO 2014/136086, which are incorporated herein by reference in their entirety.

### Non-Cationic Lipids (e.g., Phospholipids)

In some embodiments, the lipid-based carrier (or lipid nanoformulation) further comprises one or more non-cationic lipids. In some embodiments, the non-cationic lipid is a phospholipid. In some embodiments, the non-cationic lipid is a phospholipid substitute or replacement. In some embodiments, the non-cationic lipid is a negatively charged (anionic) lipid.

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), 1,2-dilauroyl-sn-glycero-3-phosphocholine (DLPC), Sodium 1,2-ditetradecanoyl-sn-glycero-3-phosphate (DMPA), phosphatidylcholine (lecithin), phosphatidylethanolamine, lysolecithin, lysophosphatidylethanolamine, phosphatidylserine, phosphatidylinositol, sphingomyelin, egg sphingomyelin (ESM), phosphatidylethanolamine (cephalin), cardiolipin, phosphatidic acid, cerebrosides, dicetylphosphate, lysophosphatidylcholine, dilinoleoylphosphatidylcholine, or mixtures thereof. It is understood that other diacylphosphatidylcholine and diacylphosphatidylethanolamine phospholipids can also be used. The acyl groups in these lipids are preferably acyl groups derived from fatty acids having C₁₀-C₂₄ carbon chains, e.g., lauroyl, myristoyl, paimitoyl, stearoyl, or oleoyl. Additional exemplary lipids, in certain embodiments, include, without limitation, those described in Kim et al. (2020) dx.doi.org/10.1021/acs.nanolett.0c01386, which is incorporated herein by reference. Such lipids include, in some embodiments, plant lipids found to improve liver transfection with mRNA (e.g., DGTS).

In some embodiments, the lipid-based carrier (or lipid nanoformulation) may comprise a combination of distearoylphosphatidylcholine/cholesterol, dipalmitoylphosphatidylcholine/cholesterol, dimyrystoylphosphatidylcholine/cholesterol, 1,2-Dioleoyl-sn-glycero-3-phosphocholine (DOPC)/cholesterol, or egg sphingomyelin/cholesterol.

Other examples of suitable non-cationic lipids include, without limitation, nonphosphorous lipids such as, e.g., stearylamine, dodecylamine, hexadecylamine, acetyl palmitate, glycerol ricinoleate, hexadecyl stearate, isopropyl myristate, amphoteric acrylic polymers, triethanolamine-lauryl sulfate, alkyl-aryl sulfate polyethyloxylated fatty acid amides, dioctadecyl dimethyl ammonium bromide, ceramide, sphingomyelin, and the like. Other non-cationic lipids are described in WO 2017/099823 or US 2018/0028664, which are incorporated herein by reference in their entirety.

In one embodiment, the lipid-based carrier (or lipid nanoformulation) further comprises one or more non-cationic lipid that is oleic acid or a compound of Formula I, II, or IV of US 2018/0028664, which is incorporated herein by reference in its entirety.

The non-cationic lipid content can be, for example, 0-30% (mol) of the total lipid components present. In some embodiments, the non-cationic lipid content is 5-20% (mol) or 10-15% (mol) of the total lipid components present.

In some embodiments, the lipid-based carrier (or lipid nanoformulation) further comprises a neutral lipid, and the molar ratio of an ionizable lipid to a neutral lipid ranges from about 2:1 to about 8:1 (e.g., about 2:1, 3:1, 4:1, 5:1, 6:1, 7:1, or 8:1).

In some embodiments, the lipid-based carrier (or lipid nanoformulation) does not include any phospholipids.

In some embodiments, the lipid-based carrier (or lipid nanoformulation) can further include one or more phospholipids, and optionally one or more additional molecules of similar molecular shape and dimensions having both a hydrophobic moiety and a hydrophilic moiety (e.g., cholesterol).

### Structural Lipids

The lipid-based carrier (or lipid nanoformulation) described herein may further comprise one or more structural lipids. As used herein, the term “structural lipid” refers to sterols (e.g., cholesterol) and also to lipids containing sterol moieties.

Incorporation of structural lipids in the lipid nanoparticle may help mitigate aggregation of other lipids in the particle. Structural lipids can be selected from the group including but not limited to, cholesterol or cholesterol derivative, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In some embodiments, structural lipids may be incorporated into the lipid-based carrier at molar ratios ranging from about 0.1 to 1.0 (cholesterol phospholipid).

In some embodiments, sterols, when present, can include one or more of cholesterol or cholesterol derivatives, such as those described in WO 2009/127060 or US 2010/0130588, which are incorporated herein by reference in their entirety. Additional exemplary sterols include phytosterols, including those described in Eygeris et al. (2020), Nano Lett. 2020; 20(6):4543-4549, incorporated herein by reference.

In some embodiments, the structural lipid is a cholesterol derivative. Non-limiting examples of cholesterol derivatives include polar analogues such as 5a-cholestanol, 53-coprostanol, cholesteryl-(2′-hydroxy)-ethyl ether, cholesteryl-(4′-hydroxy)-butyl ether, and 6-ketocholestanol; non-polar analogues such as 5a-cholestane, cholestenone, 5a-cholestanone, 5p-cholestanone, and cholesteryl decanoate; and mixtures thereof. In some embodiments, the cholesterol derivative is a polar analogue, e.g., cholesteryl-(4′-hydroxy)-butyl ether. Exemplary cholesterol derivatives are described in WO 2009/127060 and US 2010/0130588, each of which is incorporated herein by reference in its entirety.

In some embodiments, the lipid-based carrier (or lipid nanoformulation) further comprises sterol in an amount of 0-50 mol % (e.g., 0-10 mol %, 10-20 mol %, 20-50 mol %, 20-30 mol %, 30-40 mol %, or 40-50 mol %) of the total lipid components.

### Polymers and Polyethylene Glycol (PEG)—Lipids

In some embodiments, the lipid-based carrier (or lipid nanoformulation) may include one or more polymers or co-polymers, e.g., poly(lactic-co-glycolic acid) (PFAG) nanoparticles.

In some embodiments, the lipid-based carrier (or lipid nanoformulation) may include one or more polyethylene glycol (PEG) lipid. Examples of useful PEG-lipids include, but are not limited to, 1,2-Diacyl-sn-Glycero-3-Phosphoethanolamine-N-[Methoxy(Polyethylene glycol)-350](mPEG 350 PE); 1,2-Diacyl-sn-Glycero-3-Phosphoethanolamine-N-[Methoxy(Polyethylene glycol)-550](mPEG 550 PE); 1,2-Diacyl-sn-Glycero-3-Phosphoethanolamine-N-[Methoxy(Polyethylene glycol)-750](mPEG 750 PE); 1,2-Diacyl-sn-Glycero-3-Phosphoethanolamine-N-[Methoxy(Polyethylene glycol)-1000](mPEG 1000 PE); 1,2-Diacyl-sn-Glycero-3-Phosphoethanolamine-N-[Methoxy(Polyethylene glycol)-2000](mPEG 2000 PE); 1,2-Diacyl-sn-Glycero-3-Phosphoethanolamine-N-[Methoxy(Polyethylene glycol)-3000](mPEG 3000 PE); 1,2-Diacyl-sn-Glycero-3-Phosphoethanolamine-N-[Methoxy(Polyethylene glycol)-5000](mPEG 5000 PE); N-Acyl-Sphingosine-1-[Succinyl(Methoxy Polyethylene Glycol) 750](mPEG 750 Ceramide); N-Acyl-Sphingosine-1-[Succinyl(Methoxy Polyethylene Glycol) 2000](mPEG 2000 Ceramide); and N-Acyl-Sphingosine-1-[Succinyl(Methoxy Polyethylene Glycol) 5000](mPEG 5000 Ceramide). In some embodiments, the PEG lipid is a polyethyleneglycol-diacylglycerol (i.e., polyethyleneglycol diacylglycerol (PEG-DAG), PEG-cholesterol, or PEG-DMB) conjugate.

In some embodiments, the lipid-based carrier (or nanoformulation) includes one or more conjugated lipids (such as PEG-conjugated lipids or lipids conjugated to polymers described in Table 5 of WO 2019/217941, which is incorporated herein by reference in its entirety). In some embodiments, the one or more conjugated lipids is formulated with one or more ionic lipids (e.g., non-cationic lipid such as a neutral or anionic, or zwitterionic lipid); and one or more sterols (e.g., cholesterol).

The PEG conjugate can comprise a PEG-dilaurylglycerol (C12), a PEG-dimyristylglycerol (C14), a PEG-dipalmitoylglycerol (C16), a PEG-disterylglycerol (C18), PEG-dilaurylglycamide (C12), PEG-dimyristylglycamide (C14), PEG-dipalmitoylglycamide (C16), and PEG-disterylglycamide (C18).

In some embodiments, conjugated lipids, when present, can include one or more of PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy)propyl-1-0-(w-methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, and those described in Table 2 of WO 2019/051289 (which is herein incorporated by reference in its entirety), and combinations of the foregoing.

Additional exemplary PEG-lipid conjugates are described, for example, in U.S. Pat. Nos. 5,885,613, 6,287,591, US 2003/0077829, US 2003/0077829, US 2005/0175682, US 2008/0020058, US 2011/0117125, US 2010/0130588, US 2016/0376224, US 2017/0119904, US 2018/0028664, and WO 2017/099823, all of which are incorporated herein by reference in their entirety.

In some embodiments, the PEG-lipid is a compound of Formula III, III-a-I, III-a-2, III-b-1, III-b-2, or V of US 2018/0028664, which is incorporated herein by reference in its entirety. In some embodiments, the PEG-lipid is of Formula II of US 2015/0376115 or US 2016/0376224, both of which are incorporated herein by reference in their entirety. In some embodiments, the PEG-DAA conjugate can be, for example, PEG-dilauryloxypropyl, PEG-dimyristyloxypropyl, PEG-dipalmityloxypropyl, or PEG-distearyloxypropyl. In some embodiments, the PEG-lipid includes one of the following:

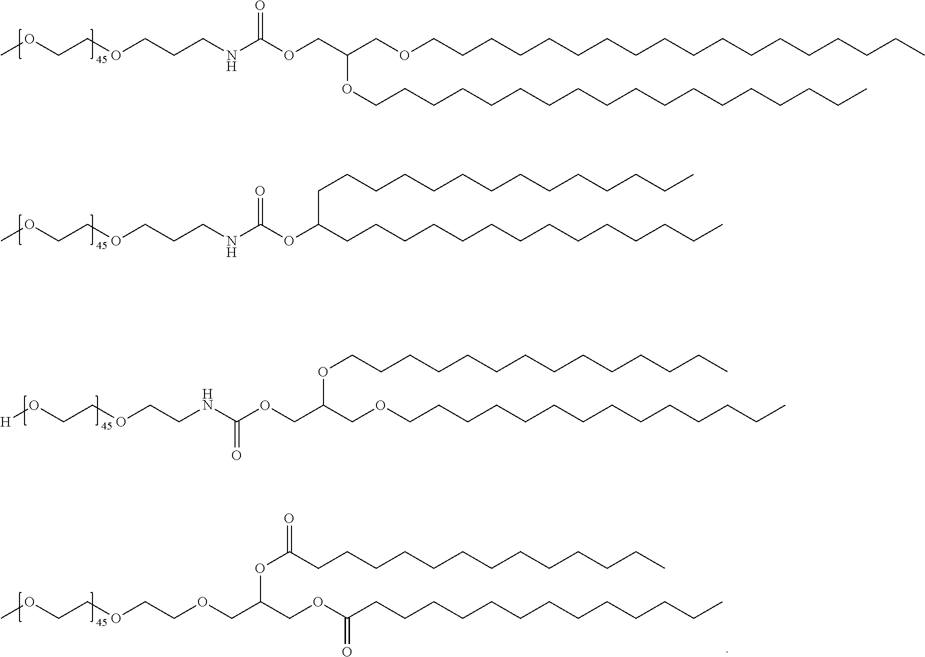

In some embodiments, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (GPL) conjugates can be used in place of or in addition to the PEG-lipid.

Exemplary conjugated lipids, e.g., PEG-lipids, (POZ)-lipid conjugates, ATTA-lipid conjugates and cationic polymer-lipids, include those described in Table 2 of WO 2019/051289A9, which is incorporated herein by reference in its entirety.

In some embodiments, the conjugated lipid (e.g., the PEGylated lipid) can be present in an amount of 0-20 mol % of the total lipid components present in the lipid-based carrier (or lipid nanoformulation). In some embodiments, the conjugated lipid (e.g., the PEGylated lipid) content is 0.5-10 mol % or 2-5 mol % of the total lipid components.

When needed, the lipid-based carrier (or lipid nanoformulation) described herein may be coated with a polymer layer to enhance stability in vivo (e.g., sterically stabilized LNPs). Examples of suitable polymers include, but are not limited to, poly(ethylene glycol), which may form a hydrophilic surface layer that improves the circulation half-life of liposomes and enhances the amount of lipid nanoformulations (e.g., liposomes or LNPs) that reach therapeutic targets. See, e.g., Working et al. J Pharmacol Exp Ther, 289: 1128-1133 (1999); Gabizon et al., J Controlled Release 53: 275-279 (1998); Adlakha Hutcheon et al., Nat Biotechnol 17: 775-779 (1999); and Koning et al., Biochim Biophys Acta 1420: 153-167 (1999), which are incorporated herein by reference in their entirety.

### Percentages of Lipid Nanoformulation Components

In some embodiments, the lipid-based carrier (or lipid nanoformulation) comprises one of more of the compounds described herein, optionally a non-cationic lipid (e.g., a phospholipid), a sterol, a neutral lipid, and optionally conjugated lipid (e.g., a PEGylated lipid) that inhibits aggregation of particles. In some embodiments, the lipid-based carrier (or lipid nanoformulation) further comprises a payload (e.g., a DNA molecule described herein). The amounts of these components can be varied independently and to achieve desired properties. For example, in some embodiments, the ionizable lipid including the lipid compounds described herein is present in an amount from about 20 mol % to about 100 mol % (e.g., 20-90 mol %, 20-80 mol %, 20-70 mol %, 25-100 mol %, 30-70 mol %, 30-60 mol %, 30-40 mol %, 40-50 mol %, or 50-90 mol %) of the total lipid components; a non-cationic lipid (e.g., phospholipid) is present in an amount from about 0 mol % to about 50 mol % (e.g., 0-40 mol %, 0-30 mol %, 5-50 mol %, 5-40 mol %, 5-30 mol %, or 5-10 mol %) of the total lipid components, a conjugated lipid (e.g., a PEGylated lipid) in an amount from about 0.5 mol % to about 20 mol % (e.g., 1-10 mol % or 5-10%) of the total lipid components, and a sterol in an amount from about 0 mol % to about 60 mol % (e.g., 0-50 mol %, 10-60 mol %, 10-50 mol %, 15-60 mol %, 15-50 mol %, 20-50 mol %, 20-40 mol %) of the total lipid components, provided that the total mol % of the lipid component does not exceed 100%.

In some embodiments, the lipid-based carrier (or lipid nanoformulation) comprises about 25-100 mol % of the ionizable lipid including the lipid compounds described herein, about 0-50 mol % phospholipid, about 0-50 mol % sterol, and about 0-10 mol % PEGylated lipid.

In some embodiments, the lipid-based carrier comprises a payload (e.g., a DNA molecule described herein, etc.) that is formulated in a lipid nanoparticle, wherein the lipid nanoparticle comprises about 25-100 mol % of the ionizable lipid including the lipid compounds described herein, about 0-50 mol % phospholipid, about 0-50 mol % sterol, and about 0-10 mol % PEGylated lipid. In some embodiments, the encapsulation efficiency of the payload may be at least 70%.

In one embodiment, the lipid-based carrier (or lipid nanoformulation) comprises about 25-100 mol % of the ionizable lipid including the lipid compounds described herein; about 0-40 mol % phospholipid (e.g., DSPC), about 0-50 mol % sterol (e.g., cholesterol), and about 0-10 mol % PEGylated lipid.

In some embodiments, the lipid-based carrier comprises a payload (e.g., a DNA molecule described herein) that is formulated in a lipid nanoparticle, wherein the lipid nanoparticle comprises about 25-100 mol % of the ionizable lipid including the lipid compounds described herein; about 0-40 mol % phospholipid (e.g., DSPC), about 0-50 mol % sterol (e.g., cholesterol), and about 0-10 mol % PEGylated lipid. In some embodiments, the encapsulation efficiency of the payload may be at least 70%.

In some embodiments, the lipid-based carrier (or lipid nanoformulation) comprises about 30-60 mol % (e.g., about 35-55 mol %, or about 40-50 mol %) of the ionizable lipid including the lipid compounds described herein, about 0-30 mol % (e.g., 5-25 mol %, or 10-20 mol %) phospholipid, about 15-50 mol % (e.g., 18.5-48.5 mol %, or 30-40 mol %) sterol, and about 0-10 mol % (e.g., 1-5 mol %, or 1.5-2.5 mol %) PEGylated lipid.

In some embodiments, the lipid-based carrier comprises a payload (e.g., a DNA molecule described herein) that is formulated in a lipid nanoparticle, wherein the lipid nanoparticle comprises about 30-60 mol % (e.g., about 35-55 mol %, or about 40-50 mol %) of the ionizable lipid including the lipid compounds described herein, about 0-30 mol % (e.g., 5-25 mol %, or 10-20 mol %) phospholipid, about 15-50 mol % (e.g., 18.5-48.5 mol %, or 30-40 mol %) sterol, and about 0-10 mol % (e.g., 1-5 mol %, or 1.5-2.5 mol %) PEGylated lipid. In some embodiments, the encapsulation efficiency of the payload may be at least 70%.

In some embodiments, molar ratios of ionizable lipid/sterol/phospholipid (or another structural lipid)/PEG-lipid/additional components is varied in the following ranges: ionizable lipid (25-100%); phospholipid (DSPC) (0-40%); sterol (0-50%); and PEG lipid (0-5%).

In some embodiments, the lipid-based carrier comprises a payload (e.g., a DNA molecule described herein) that is formulated in a lipid nanoparticle, wherein the lipid nanoparticle comprises molar ratios of ionizable lipid/sterol/phospholipid (or another structural lipid)/PEG-lipid/additional components in the following ranges: ionizable lipid (25-100%); phospholipid (DSPC) (0-40%); sterol (0-50%); and PEG lipid (0-5%). In some embodiments, the encapsulation efficiency of the payload may be at least 70%.

In some embodiments, the lipid-based carrier (or lipid nanoformulation) comprises, by mol % or wt % of the total lipid components, 50-75% ionizable lipid (including the lipid compound as described herein), 20-40% sterol (e.g., cholesterol or derivative), 0 to 10% non-cationic-lipid, and 1-10% conjugated lipid (e.g., the PEGylated lipid).

In some embodiments, the lipid-based carrier comprises a payload (e.g., a DNA molecule described in) that is formulated in a lipid nanoparticle, wherein the lipid nanoparticle comprises, by mol % or wt % of the total lipid components, 50-75% ionizable lipid (including the lipid compound as described herein), 20-40% sterol (e.g., cholesterol or derivative), 0 to 10% non-cationic-lipid, and 1-10% conjugated lipid (e.g., the PEGylated lipid). In some embodiments, the encapsulation efficiency of the payload may be at least 70%.

In some embodiments, the lipid-based carrier (or lipid nanoformulation) comprises (i) a DNA molecule; (ii) a cationic lipid comprising from 50 mol % to 65 mol % of the total lipid present in the lipid-based carrier; (iii) a non-cationic lipid comprising a mixture of a phospholipid and a cholesterol derivative thereof, wherein the phospholipid comprises from 3 mol % to 15 mol % of the total lipid present in the lipid-based carrier and the cholesterol or derivative thereof comprises from 30 mol % to 40 mol % of the total lipid present in the lipid-based carrier; and (iv) a conjugated lipid comprising 0.5 mol % to 2 mol % of the total lipid present in the particle.

In some embodiments, the lipid-based carrier (or lipid nanoformulation) comprises (i) a DNA molecule; (ii) a cationic lipid comprising from 50 mol % to 85 mol % of the total lipid present in the lipid-based carrier; (iii) a non-cationic lipid comprising from 13 mol % to 49.5 mol % of the total lipid present in the lipid-based carrier; and (d) a conjugated lipid comprising from 0.5 mol % to 2 mol % of the total lipid present in the lipid-based carrier.

In some embodiments, the phospholipid component in the mixture may be present from 2 mol % to 20 mol %, from 2 mol % to 15 mol %, from 2 mol % to 12 mol %, from 4 mol % to 15 mol %, from 4 mol % to 10 mol %, from 5 mol % to 10 mol %, (or any fraction of these ranges) of the total lipid components. In some embodiments, the lipid-based carrier (or lipid nanoformulation) is phospholipid-free.

In some embodiments, the sterol component (e.g. cholesterol or derivative) in the mixture may comprise from 25 mol % to 45 mol %, from 25 mol % to 40 mol %, from 25 mol % to 35 mol %, from 25 mol % to 30 mol %, from 30 mol % to 45 mol %, from 30 mol % to 40 mol %, from 30 mol % to 35 mol %, from 35 mol % to 40 mol %, from 27 mol % to 37 mol %, or from 27 mol % to 35 mol % (or any fraction of these ranges) of the total lipid components.

In some embodiments, the non-ionizable lipid components in the lipid-based carrier (or lipid nanoformulation) may be present from 5 mol % to 90 mol %, from 10 mol % to 85 mol %, or from 20 mol % to 80 mol % (or any fraction of these ranges) of the total lipid components.

The ratio of total lipid components to the payload (e.g., an encapsulated therapeutic agent such as a DNA molecule) can be varied as desired. For example, the total lipid components to the payload (mass or weight) ratio can be from about 10:1 to about 30:1. In some embodiments, the total lipid components to the payload ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of total lipid components and the payload can be adjusted to provide a desired N/P ratio, for example, N/P ratio of 3, 4, 5, 6, 7, 8, 9, 10, 11, 12, 13, 14, 15, 16, 17, 18, 19, 20, 21, 22, 23, 24, 25, 26, 27, 28, 29, 30, or higher. Generally, the lipid-based carrier (or lipid nanoformulation's) overall lipid content can range from about 5 mg/ml to about 30 mg/mL. Nitrogen:phosphate ratios (N:P ratio) is evaluated at values between 0.1 and 100.

The efficiency of encapsulation of a payload such as a DNA molecule, describes the amount of the DNA molecule that is encapsulated or otherwise associated with a lipid nanoformulation (e.g., liposome or LNP) after preparation, relative to the initial amount provided. The encapsulation efficiency is desirably high (e.g., at least 70%, 80%, 90%, 95%, or close to 100%). The encapsulation efficiency may be measured, for example, by comparing the amount of DNA molecule in a solution containing the liposome or LNP before and after breaking up the liposome or LNP with one or more organic solvents or detergents. An anion exchange resin may be used to measure the amount of DNA molecule in a solution. Fluorescence may be used to measure the amount of DNA molecule in a solution. For the lipid-based carrier (or lipid nanoformulation) described herein, the encapsulation efficiency of a DNA molecule may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 70%. In some embodiments, the encapsulation efficiency may be at least 80%. In some embodiments, the encapsulation efficiency may be at least 90%. In some embodiments, the encapsulation efficiency may be at least 95%.

### Route of Administration

In some aspects, a composition described herein, e.g., a tLNP composition comprising a DNA molecule described herein, is introduced into a cell, tissue or subject by any suitable route.

Administration to a target cell or tissue (e.g., ex vivo) may be by methods known in the art such as transfection, e.g., transient or stable transfection using reagents (e.g., liposomal, calcium phosphate) or physical means (e.g., electroporation, gene gun, microinjection, microfluidic fluid shear, cell squeezing). Other methods are described, e.g., in Rad et al. 2021. Adv. Mater. 33:2005363, which is incorporated herein by reference.

Administration to a subject, e.g., a mammal, e.g., a human subject, may be by parenteral (e.g., intravenous, intramuscular, intraperitoneal, subcutaneous, or intracranial) route; by topical administration, transdermal administration or transcutaneous administration. Other suitable routes include oral, rectal, transmucosal, intranasal, inhalation (e.g., via an aerosol), buccal (e.g., sublingual), vaginal, intrathecal, intraocular, transdermal, intraendothelial, in utero (or in ovo), intrapleural, intracerebral, intraarticular, topical, intralymphatic. Also included is direct tissue or organ injection (e.g., to liver, eye, skeletal muscle, cardiac muscle, diaphragm, muscle or brain).

### Applications

A composition described herein, e.g., a tLNP composition comprising a DNA molecule described herein, can be used in therapeutic or health applications for a subject, e.g., a human or non-human animal. Although the descriptions of pharmaceutical compositions provided herein are principally directed to pharmaceutical compositions which are suitable for administration to humans, it will be understood by the skilled artisan that such compositions are generally suitable for administration to any other animal. The subject can be any animal, e.g., a mammal, e.g., a human or non-human mammal. In embodiments, the subject is a vertebrate animal (e.g., mammal, bird, fish, reptile, or amphibian). In embodiments, the subject is a human. In embodiments, the method subject is a non-human mammal. In embodiments, the subject is a non-human mammal is such as a non-human primate (e.g., monkeys, apes), ungulate (e.g., cattle, buffalo, sheep, goat, pig, camel, llama, alpaca, deer, horses, donkeys), carnivore (e.g., dog, cat), rodent (e.g., rat, mouse), or lagomorph (e.g., rabbit). In embodiments, the subject is a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots). In embodiments, the subject is an invertebrate such as an arthropod (e.g., insects, arachnids, crustaceans), a nematode, an annelid, a helminth, or a mollusk.

In some aspects, the present disclosure provides a method of treating a disease or disorder in a subject, comprising administering to the subject a composition described herein, e.g., a composition comprising an effector sequence that encodes a CAR. In some embodiments, the disease or disorder comprises a cancer. In some embodiments, the cancer is a hematological cancer. In some embodiments, the cancer is a lymphoma (e.g., non-Hodgkin lymphoma (NHL)), leukemia, or multiple myeloma. In some embodiments, the cancer is a diffuse large B-cell lymphoma (DLBCL), mantle cell lymphoma (MCL), follicular lymphoma, or acute lymphoblastic leukemia (ALL), chronic lymphocytic leukemia (CLL), small lymphocytic lymphoma (SLL), or acute myeloid leukemia (AML). In some embodiments, the disease or disorder comprises an autoimmune disease. In some embodiments, the autoimmune disease is lupus (e.g., systemic lupus erythematosus (SLE)), systemic sclerosis, idiopathic inflammatory myopathy, dermatomyositis, or myasthenia gravis.

In some embodiments, a DNA molecule described herein is provided at a dose of about 0.1-100 mg/kg of DNA.

In some embodiments, a nucleic acid molecule (e.g., a DNA molecule and/or an RNA molecule) is provided at a dose of 5 μg to 300 μg nucleic acid molecules, e.g., when administered via intramuscular route. In some embodiments, a nucleic acid molecule (e.g., a DNA molecule and/or an RNA molecule) is provided at a dose of 0.001 mg/kg to 2 mg/kg nucleic acid molecules, e.g., when administered via intravenous route.

In some embodiments, a DNA molecule described herein imparts a biological effect of the effector, e.g., expression of a therapeutic polypeptide, on a host cell, tissue or subject over a time period of at least 2, at least 3, at least 4, at least 5, at least 6 days or at least a week; at least 8, at least 9, at least 10, at least 12, at least 14 days or at least two weeks; at least 16, at least 18, at least 20 days or at least 3 weeks; at least 22, at least 24, at least 25, at least 27, at least 28 days or at least a month; at least 2 months, at least 3 months, at least 4 months, at least 5 months, at least 6 months or more; between one week and 6 months, between 1 month to 6 months, or between 3 months to 6 months.

In some embodiments, a DNA molecule described herein imparts a biological effect of the effector, e.g., expression of a therapeutic polypeptide, on a host cell, tissue or subject over a time period of at least 1 cell divisions of the host cell.

In embodiments, a DNA molecule described herein can be used to deliver an effector, e.g., an effector described herein, to a cell, tissue or subject.

In embodiments, a DNA molecule described herein can be used to modulate (e.g., increase or decrease) a biological parameter in a cell, tissue or subject. The biological parameter may be an increase or decrease in gene expression of a subject gene in a target cell, tissue or subject. In some embodiments, a DNA molecule described herein increases or decreases a biological activity in a target cell, wherein the biological activity comprises cell growth, cell metabolism, cell signaling, cell movement, specialization, interactions, division, transport, homeostasis, osmosis, or diffusion. In some embodiments, the cell is an animal cell, e.g., a mammalian cell, e.g., a human cell.

In embodiments, a DNA molecule described herein can be used to treat a cell, tissue or subject in need thereof by administering the DNA molecule described herein to such cell, tissue or subject.

In embodiments, the DNA molecule delivers an effector to a cell.

In some embodiments, a tLNP composition described herein can be used to treat a disease or disorder in a tissue or subject in need thereof by administering the tLNP composition described herein to such tissue or subject. In some embodiments, the tLNP composition comprises an LNP, a targeting moiety on the surface of the LNP, wherein the targeting moiety binds a T cell antigen, and a dsDNA molecule comprising an effector sequence that encodes an effector. In some embodiments, the effector is a CAR. In some embodiments, the disease or disorder is a cancer. In some embodiments, the CAR targets the cancer of the subject. In some aspects, the present disclosure provides a composition described herein, e.g., a tLNP composition described herein, for use in treating a disease or disorder in a tissue or subject in need thereof. In some aspects, the present disclosure provides use of a composition described herein, e.g., a tLNP composition described herein, in treating a disease or disorder in a tissue or subject in need thereof. In some aspects, the present disclosure provides use of a composition described herein, e.g., a tLNP composition described herein, in the manufacture of a medicament for treating a disease or disorder in a tissue or subject in need thereof.

### EXAMPLES

### Example 1: Activation of Patient-Derived T Cells

This Example demonstrates the activation of primary, patient-derived T cells.

Cryopreserved human primary T cells isolated from leukopaks were quickly thawed at 37° C., followed by dilution in medium (ImmunoCult™-XF T Cell Expansion Medium, 10981, Stemcell Technologies) supplemented with 10 ng/mL human recombinant IL-2 (78036.3, Stemcell Technologies) and 1% 5,000 U/mL Penicillin/Streptomycin (15070-063, Thermo Fisher) and centrifugation for 5 minutes at 1,000 rpm at room temperature. The cell pellet was resuspended in complete medium for a cell concentration of ~1E6 cells/mL and activated by addition of 5 uL/mL of CD3/CD28 T Cell Activator (10991, Stemcell Technologies) in tissue culture flasks. The cells were incubated at 37° C.+5% CO₂ with daily monitoring for cell numbers and viability to maintain the culture at approximately 500,000 cells/mL before use for targeted LNP (tLNP) transfection experiments at Day 3 post activation.

### Example 2: Transfection of T Cells with tLNPs

This Example demonstrates tLNP transfection of T cells, e.g., primary patient-derived T cells.

tLNPs were formulated by encapsulating circular “hemi-modified” dsDNA molecules containing chemically modified nucleobases on the sense strand. The dsDNA molecules were produced in a reaction which included complete incorporation of either 5-hydroxymethyluracil or canonical uracil, in place of thymine, on the sense strand. The dsDNA molecules comprise a sequence encoding a reporter protein (GFP) driven by a ubiquitin C (UBC) promoter, a 5′ UTR, a 3′ UTR, and a sequence encoding a bovine growth hormone (bGH) polyadenylation signal.

In brief, the dsDNA molecules were diluted into citrate buffer (pH 4.0). Lipids, including a commercially available ionizable lipid, as well as DSPC, cholesterol, DMG-PEG2000 and DSPE-PEG2000-DBCO at a mol ratio of 50/10/38.5/1/0.5, were mixed into ethanol. The resulting dsDNA and lipid solutions were mixed at a ratio of 3:1 dsDNA:lipids (vol:vol) and a nitrogen:phosphate (N/P) ratio of 6. The LNPs were washed and concentrated using Amicon centrifugal filter units (100 kDa, UFC8100, Millipore). An anti-CD3 antibody, comprising the sequences in Table 2, was conjugated to the DSPE-PEG2000-DBCO lipid by click chemistry for targeting to T cells.

TABLE 2
Sequences of an anti-CD3 antibody molecule.

| Region | Sequence |
|---|---|
| Heavy chain | QVQLQQSGAELARPGASVKMSCKASGYTFTRYTM |
| variable | HWVKQRPGQGLEWIGYINPSRGYTNYNQKFKDKA |
| region (VH) | TLTTDKSSSTAYMQLSSLTSEDSAVYYCARYYDDHY |
| | CLDYWGQGTTLTVSS (SEQ ID NO: 2) |
| Light chain | QIVLTQSPAIMSASPGEKVTMTCSASSSVSYMNWY |
| variable | QQKSGTSPKRWIYDTSKLASGVPAHFRGSGSGTSYS |
| region (VL) | LTISGMEAEDAATYYCQQWSSNPFTFGSGTKLEIN |
| | (SEQ ID NO: 3) |

Primary T cells pre-activated as described in Example 1, as well as naïve T cells, were pelleted then resuspended in fresh complete medium to seed 50,000 cells in 50 uL of complete medium per well in a 96-well V-bottom plate. 50 uL of media containing 2× the final concentration needed of tLNPs were added to the corresponding wells and incubated at 37° C.+5% CO₂ before harvesting at the indicated time points.

### Example 3: Measurement of Effector Expression Following tLNP Delivery

This Example demonstrates expression of an effector following delivery of T cell-targeted tLNPs.

Primary T cells from three different donors were pre-activated as described in Example 1, and then transfected with tLNPs (comprising lipid conjugated to an anti-CD3 antibody) as described in Example 2. The tLNPs were contacted with the pre-activated T cells at a final concentration of 2 μg/mL (“tLNP low”), 8 μg/mL (“tLNP med”), or 16 μg/mL (“tLNP high”) in the cell culture media. At 48 hours following transfection, the percentage of GFP+ T cells was measured via flow cytometry.

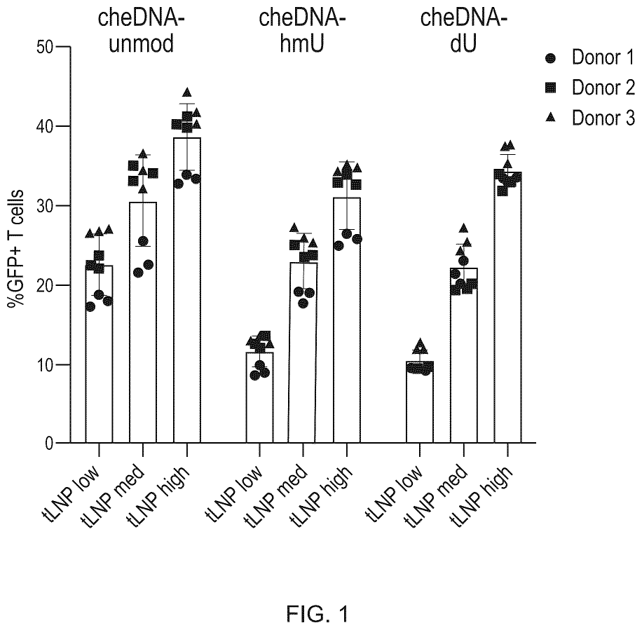

As shown in FIG. 1, transfection of primary activated T cells with tLNPs comprising circular dsDNA molecules lacking chemically modified nucleobases (“cheDNA-unmod”), as well as circular “hemi-modified” dsDNA molecules comprising either 5-hydroxymethyluracil (“cheDNA-hmU”) or canonical uracil (“cheDNA-dU”) on the sense strand resulted in expression of GFP. The expression of GFP was observed across the multiple T cell donors and at various tLNP concentrations.

Naïve T cells (cells not contacted with a cell activator of Example 1) from three separate donors were transfected with tLNPs (comprising lipid conjugated to an anti-CD3 antibody) as described in Example 2. The tLNPs were transfected for a final concentration of 4 μg of DNA/mL of media. At 2 days and 4 days following transfection, the percentage of GFP+ T cells was measured via flow cytometry.

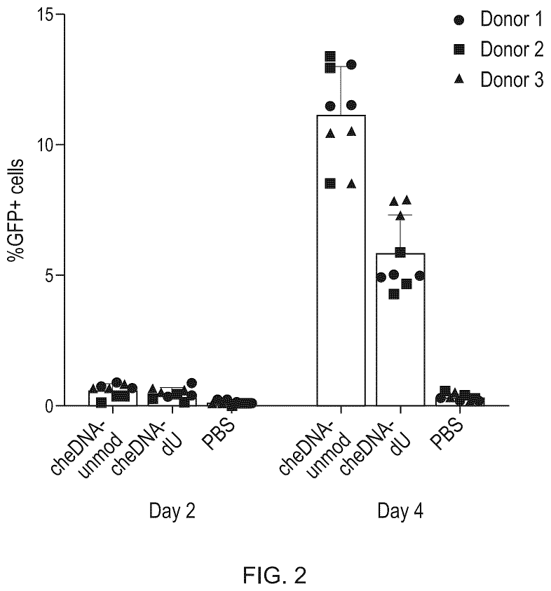

As shown in FIG. 2, transfection of primary naïve T cells with circular dsDNA molecules, including circular dsDNA molecules lacking chemically modified nucleobases (“cheDNA-unmod”) and circular “hemi-modified” dsDNA molecules comprising canonical uracil on the sense strand (“cheDNA-dU”), resulted in GFP expression, including at day 4 post-transfection, and the GFP expression was observed across the multiple T cell donors.

These results demonstrate that tLNPs comprising circular dsDNA molecules, including circular “hemi-modified” dsDNA molecules comprising chemically modified nucleobases on the sense strand, encapsulated by lipid conjugated with a T cell targeting moiety, can be transfected into primary pre-activated and naïve T cells, and the circular dsDNA molecules can be transcribed and ultimately yield a protein product in the T cells.

### Example 4: Measurement of T Cell Innate Immune Responses

This Example demonstrates measurement of the innate immune response in T cells following transfection of T cell-targeted tLNPs. In some embodiments, it is desirable to minimize the T cell's innate immune response in response to exogenous DNA.

Primary T cells from three separate donors were pre-activated as described in Example 1, and transfected with tLNPs (comprising lipid conjugated to an anti-CD3 antibody) as described in Example 2. The tLNPs were transfected for a final concentration of 2 to 16 μg of DNA/mL of media. As a negative control for innate immune response stimulation, tLNPs comprising mRNA molecules that encode GFP and comprise N1-psuedomethyluridine modifications were transfected into pre-activated T cells. At 24 hours following transfection, the levels of 2′-3′ cGAMP were measured by ELISA using the 2′3′-Cyclic GAMP ELISA Kit (ThermoFisher #EIAGAMP), and the levels of a panel of cytokines were measured using the LEGENDplex™ Hu Anti-Virus Response Panel 1 (13-plex) w/VbP V02 (Catalog #741270).

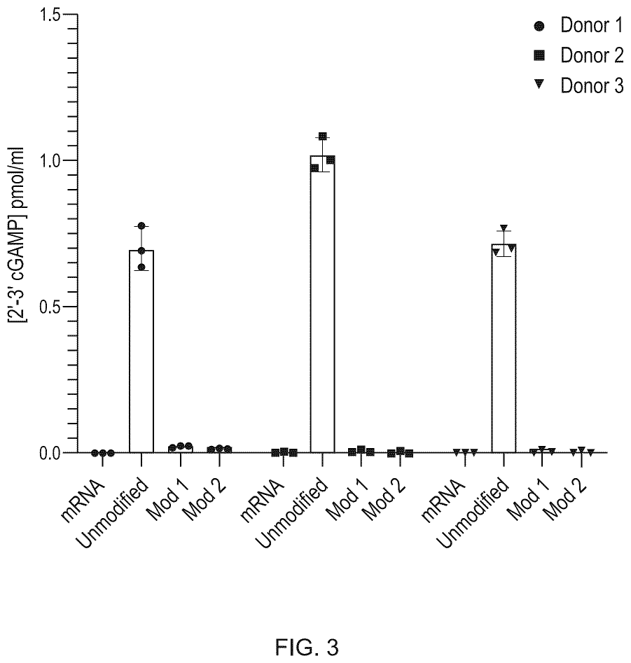

As shown in FIG. 3, transfection of tLNPs comprising circular “hemi-modified” dsDNA molecules with either 5-hydroxymethyluracil (“Mod 1”) or canonical uracil (“Mod 2”) on the sense strand resulted in lower 2′-3′ cGAMP levels as compared to tLNPs comprising circular dsDNA molecules lacking chemically modified nucleobases (“Unmodified”), indicating that incorporation of chemical modifications in the sense strand of circular dsDNA abrogates cGAMP induction across multiple T cell donors. In addition, it was observed that introduction of T cell-targeting tLNPs induced expression of several cytokines. For certain cytokines, in particular IFN-λ and IP10, their protein levels were decreased in T cells contacted with tLNPs comprising circular “hemi-modified” dsDNA molecules (with either 5-hydroxymethyluracil or canonical uracil on the sense strand) as compared to tLNPs comprising circular dsDNA molecules that lacked chemically modified nucleobases.

In summary, this data suggests that incorporation of chemically modified nucleobases into the sense strand of circular dsDNA molecules in T cell-targeted tLNPs decreased the innate immune response to the dsDNA in T cells, including abrogration of cGAMP induction and suppression of stimulation of certain cytokines.

For all patents, applications, or other reference cited herein, such as non-patent literature and reference sequence information, it should be understood that they are incorporated by reference in their entirety for all purposes as well as for the proposition that is recited. Where any conflict exists between a document incorporated by reference and the present application, this application will control. All information associated with reference gene sequences disclosed in this application, such as GeneIDs or accession numbers (typically referencing NCBI accession numbers), including, for example, genomic loci, genomic sequences, functional annotations, allelic variants, and reference mRNA (including, e.g., exon boundaries or response elements) and protein sequences (such as conserved domain structures), as well as chemical references (e.g., PubChem compound, PubChem substance, or PubChem Bioassay entries, including the annotations therein, such as structures and assays, et cetera), are hereby incorporated by reference in their entirety.

Headings used in this application are for convenience only and do not affect the interpretation of this application.

1. A targeted lipid nanoparticle (tLNP) composition comprising:
a) a lipid nanoparticle (LNP),
b) a targeting moiety on the surface of the LNP, wherein the targeting moiety binds a T cell antigen, and
c) a double stranded DNA molecule comprising an effector sequence that encodes an effector.

2. The tLNP composition of claim 1, wherein the DNA molecule is circular, or the DNA molecule comprises:
(i) an upstream DNA end form which is a closed end;
(ii) a double stranded region; and
(iii) a downstream DNA end form which is a closed end.

3. (canceled)

4. The tLNP composition of claim 1, wherein the targeting moiety comprises an antibody or a ligand.

5. The tLNP composition of claim 1, wherein the targeting moiety comprises an anti-CD3 antibody or an anti-CD7 antibody.

6. The tLNP composition of claim 1, wherein the targeting moiety is connected to the LNP via a click linker.

7. The tLNP composition of claim 1, wherein the DNA molecule comprises a promoter sequence operably linked to the effector sequence.

8. The tLNP composition of claim 1, wherein the effector comprises a polypeptide, an RNA, a DNA binding protein, an antigen, an epigenetic modifying factor, a hormone, an enzyme, a nuclease element of a CRISPR system, a mobile genetic element protein, a gene writer, an antibody, a signaling peptide, a receptor ligand, a receptor, a clotting factor, or a chimeric antigen receptor (CAR).

9.-14. (canceled)

15. The tLNP composition of claim 1, wherein the LNP comprises a cationic lipid, a non-cationic lipid, a structural lipid, or a PEG-modified lipid.

16.-18. (canceled)

19. A pharmaceutical composition comprising the tLNP composition of claim 1.

20. A method of introducing a DNA molecule into a T cell, the method comprising:
(a) providing a cell population comprising the T cell;
(b) contacting the cell population with the tLNP composition of claim 1,
thereby introducing the DNA molecule into the T cell.

21. A method of treating a disease or disorder in a subject, the method comprising:
administering to the subject the tLNP composition of claim 1;
thereby treating the disease or disorder.

22.-23. (canceled)

24. A method of making a tLNP composition, the method comprising:
(I) (a) providing a double stranded DNA molecule; and
(b) contacting the double stranded DNA molecule with a lipid, wherein the lipid is linked to a targeting moiety that binds a T cell antigen;
(II) (a) providing a lipid covalently linked to a first click handle;
(b) providing a targeting moiety that binds a T cell antigen, wherein the targeting moiety is covalently linked to a second click handle; and
(c) contacting (a) with (b) under conditions that allow for reaction of the first click handle with the second click handle, thereby producing a click linker between the lipid and the targeting moiety; and
(d) contacting the lipid and the targeting moiety with a double stranded DNA molecule; or
(III) (a) providing a lipid covalently linked to a first click handle;
(b) contacting the lipid with a double stranded DNA molecule, thereby forming an LNP composition; and
(c) contacting the LNP composition with a targeting moiety that binds a T cell antigen, wherein the targeting moiety is covalently linked to a second click handle, under conditions that allow for reaction of the first click handle with the second click handle, thereby producing a click linker between the lipid and the targeting moiety;
thereby making the tLNP composition.

25.-27. (canceled)

28. A method of making or preparing a pharmaceutical composition, the method comprising:
(a) providing a test batch comprising an LNP, a test targeting moiety that binds a T cell antigen on the surface of the LNP, and a double stranded DNA molecule comprising an effector sequence that encodes an effector;
(b) measuring or having measured one or more of:
(i) a level of binding to a T cell antigen in a sample of the test batch;
(ii) a level of binding to a T cell in a sample of the test batch;
(iii) a molar ratio of the test targeting moiety to the LNP in a sample of the test batch;
(iv) a molar ratio of the test targeting moiety to a selected lipid in the LNP in a sample of the test batch;
(v) expression of the effector in a cell having the T cell antigen, wherein the cell has been contacted with a sample of the test batch; or
(vi) potency of a sample of the test batch; and
(c) if the measurement of (b) is equal to or greater than a pre-determined threshold, or if the measurement of (b) falls within a pre-determined range, then performing or having performed one or more of:
(i) formulating or having formulated the test batch as a pharmaceutical composition;
(ii) dividing the test batch or pharmaceutical composition into a plurality of portions;
(iii) contacting the test batch or pharmaceutical composition, or a portion thereof, with an excipient;
(iv) placing the test batch or pharmaceutical composition, or a portion thereof, in a container;
(v) labeling a container comprising the test batch or pharmaceutical composition, or a portion thereof;
(vi) distributing the test batch or pharmaceutical composition, or a portion thereof;
(vii) storing the test batch or pharmaceutical composition, or a portion thereof;
(viii) releasing the test batch or pharmaceutical composition, or a portion thereof, into commerce; or
(ix) performing a pharmaceutical release specification.

29. The tLNP composition of claim 1, which further comprises an RNA molecule.

30.-32. (canceled)

33. A method of introducing a DNA molecule and an RNA molecule into a T cell, the method comprising:
(a) providing a cell population comprising the T cell;
(b) contacting the cell population with the tLNP composition of claim 29,
thereby introducing the DNA molecule and the RNA molecule into the T cell.

34.-37. (canceled)

38. The tLNP composition of claim 1, wherein the DNA molecule comprises a first strand and a second strand, wherein the first strand comprises one or more chemically modified nucleobase and the second strand is free of chemically modified nucleobases.

39. The tLNP composition of claim 38, wherein the first strand is a sense strand and the second strand is an antisense strand.

40. The tLNP composition of claim 38, wherein the first strand is an antisense strand and the second strand is a sense strand.

41. The tLNP composition of claim 38, wherein:
(a) at least 20% of positions in the first strand of the DNA molecule comprise chemically modified nucleobases;
(b) the chemically modified nucleobase comprises a uracil nucleobase; or
(c) at least 90% of thymine or uracil positions in the first strand of the DNA molecule comprise a uracil nucleobase.

42. The tLNP composition of claim 41, wherein the uracil nucleobase is a canonical uracil nucleobase or a chemically modified uracil nucleobase, optionally wherein the chemically modified uracil nucleobase comprises 5-hydroxymethyluracil.
